(19) United States
(12) Patent Application Publication
(10) Pub. No.: US 2013/0237948 A1
(43) Pub. Date: Sep. 12, 2013

(54) DEVICES FOR REGULATION OF BLOOD PRESSURE AND HEART RATE

(71) Applicant: EnteroMedics, Inc., St. Paul, MN (US)

(72) Inventors: Adrianus P. Donders, Andover, MN (US); Katherine S. Tweden, Mahtomedi, MN (US); Mark B. Knudson, Shoreview, MN (US); Arnold W. Thornton, Roseville, MN (US)

(21) Appl. No.: 13/784,235

(22) Filed: Mar. 4, 2013

Related U.S. Application Data
(60) Provisional application No. 61/607,701, filed on Mar. 7, 2012.

(51) Int. Cl. A61N 1/36

(52) U.S. Cl. CPC A61N 1/36114
USPC 604/500; 607/59; 607/44; 607/30; 29/592.1

(57) ABSTRACT

A method and apparatus for treating a condition associated with impaired blood pressure and/or heart rate in a subject comprising applying an electrical treatment signal, wherein the electrical treatment signal is selected to at least partially block nerve impulses, or in some embodiments, to augment nerve impulses. In embodiments, the apparatus provides a first therapy program to provide a downregulating signal to one or more nerves including renal artery, renal nerve, vagus nerve, celiac plexus, a splanchnic nerve, cardiac sympathetic nerves, spinal nerves originating between T10 to L5. In embodiments, the apparatus provides a third therapy program to provide an upregulating signal to one or more nerves including a glossopharyngeal nerve and/or a tissue containing baroreceptors.

### CROSS REFERENCE

This Application claims priority to U.S. application No. 61/607,701, filed Mar. 7, 2012, which application is hereby incorporated by reference.

### BACKGROUND OF THE INVENTION

It is estimated that approximately 50 million people in the US have high blood pressure. The criteria for diagnosis of hypertension has changed: a blood pressure of 120/80 mmHg is considered normal; 120-139 over 80-89 mmHg is defined as pre-hypertensive; greater than or equal to 140-159 mmHg systolic over 90-99 mmHg diastolic is stage I hypertension; and greater than or equal to 160 mmHg systolic over greater than or equal to 100 mmHg diastolic is stage II hypertension. (The Seventh Report of the Joint National Committee on Prevention, Detection, Evaluation and Treatment of High Blood Pressure, (JNC 7), NHLBI publication, Hypertension 42:1206, 2003). Of those who have been diagnosed, about two thirds do not achieve blood pressure control of less than 140/90 mm Hg, and nearly 15% receive no treatment at all. About half of the people with hypertension never know they have high blood pressure because of the lack of specific symptoms. In most cases of hypertension, the cause is unknown, so the diagnosis is called primary hypertension. In about 5 to 10 percent of people, high blood pressure is a secondary symptom of some other medical condition. For example, there might be an organic cause such as kidney disease, tumor of the adrenal glands, heart defects, or disorders of the nervous apparatus.

Aggressive drug treatment of long-term high blood pressure can significantly reduce the incidence of death from heart disease and other causes in both men and women. In people with diabetes, controlling both blood pressure and blood glucose levels prevents serious complications of that disease. If patients have mild hypertension and no heart problems, then lifestyle changes may suffice to control the condition. For more severe hypertension or for mild cases that do not respond to changes in diet and lifestyle within a year, drug treatment is usually necessary. A single-drug regimen is usual to control mild to moderate hypertension. More severe hypertension often requires a combination of two or more drugs. Prolonged-release drugs are being developed so that they are most effective during early morning periods, when patients are at highest risk for heart attack or stroke.

It is very important to rigorously maintain a drug regimen. Patients who discontinue antihypertensive therapy, particularly smokers and younger adults, are at a significantly increased risk for stroke. All drugs used for hypertension have side effects. Common side effects include fatigue, coughing, skin rash, sexual dysfunction, depression, cardiac dysfunction, or electrolyte abnormalities. Because of these side effects finding the best drug for the patient while encouraging ongoing patient compliance may be difficult.

Congestive heart failure (CHF) is a condition where the heart pump efficiency (cardiac output) of the heart becomes so low that blood circulation is inadequate to meet tissue needs. Congestive heart failure is usually a progressively worsening condition resulting in serious disability and death. Approximately five million Americans, with a significant percentage being under the age of 60 years, suffer from CHF. Past research suggests that a slowing an elevated heart rate can improve heart performance.

Despite the availability of many therapies, hypertension and congestive heart failure remain major health issues. Many of the therapies have undesirable side effects, or do not achieve adequate control of blood pressure or heart rate. Thus, there remains a need to develop devices and methods for regulating blood pressure and/or heart rate.

### SUMMARY

This disclosure provides devices and methods for treating conditions relating to impaired blood pressure, heart rate control, metabolic disease, and/or chronic kidney disease. In embodiments, a method of treating a condition associated with impaired heart rate, blood pressure, and/or chronic kidney disease in a subject comprises applying an intermittent electrical treatment signal to a target nerve or tissue in proximity to the target nerve of the subject, wherein said electrical treatment signal is selected to at least partially modulate neural activity on the nerve during an on time and to at least partially restore neural activity on the nerve during an off time. In specific embodiments, a method is applied to treat hypertension, congestive heart failure, chronic renal disease, metabolic disease, metabolic syndrome, sleep apnea, and cardiovascular disease.

In embodiments, an apparatus comprises a first electrode adapted to be placed on a first target nerve or blood vessel such as a renal artery, renal nerve, celiac plexus, a splanchnic nerve, cardiac sympathetic nerves, and spinal nerves originating between T10 to L5; an implantable neuroregulator connected to the electrodes and configured to deliver a first therapy program to the first target nerve or blood vessel, wherein the first therapy program delivers an electrical signal to the first target nerve or blood vessel intermittently with an on time and an off time multiple times in a day, wherein the first therapy program delivers an electrical signal treatment that has a frequency selected to down regulate neural activity on the first nerve or blood vessel during an on time and has an off time selected to provide for at least partial recovery of nerve function; and an external coil, wherein the external coil is configured to communicate data and power signals to the neuroregulator and to communicate data to another programming device.

In other embodiments, an apparatus comprises an additional electrode adapted to be placed on a second target nerve or blood vessel such as a renal artery, renal nerve, vagus nerve, celiac plexus, a splanchnic nerve, and cardiac sympathetic nerves, spinal nerves originating between T10 to L5, glossopharyngeal nerve, and tissue containing baroreceptors. In embodiments, the first and additional electrodes are each placed on the same nerve or different nerves.

In another embodiment, when the additional electrode is adapted to be placed on a second target nerve or tissue selected from renal artery, renal nerve, vagus nerve, celiac plexus, a splanchnic nerve, cardiac sympathetic nerves, spinal nerves originating between T10 to L5, the implantable neuroregulator is configured to deliver the first therapy program to the second target nerve or tissue.

In another embodiment, when the additional electrode is adapted to be placed on a second target nerve or tissue selected from renal artery, renal nerve, vagus nerve, celiac plexus, a splanchnic nerve, cardiac sympathetic nerves, spinal nerves originating between T10 to L5, the implantable neuroregulator is configured to deliver a first therapy program to the first target nerve or tissue, and a second therapy program to the second target nerve or tissue, where each therapy program delivers an electrical signal treatment that has a frequency selected to down regulate neural activity on the first nerve or blood vessel and/or the second nerve or blood vessel during an on time and has an off time selected to provide for at least partial recovery of nerve function; and an external coil, wherein the external coil is configured to communicate data and power signals to the neuroregulator and to communicate data to another programming device.

In a further embodiment, an apparatus further comprises when the additional electrode is adapted to be placed on a second target nerve or tissue selected from a glossopharyngeal nerve, tissue containing baroreceptors, and combinations thereof, the implantable neuroregulator is configured to deliver a third therapy program to the second target nerve or tissue, wherein the third therapy program delivers an electrical signal to second target nerve or blood vessel intermittently with an on time and an off time multiple times in a day, wherein the third therapy program delivers an electrical signal treatment that has a frequency to up regulate neural activity.

In other embodiments, an apparatus is a closed loop apparatus and further comprises a sensor. The sensor can measure heart rate, blood pressure, mean arterial pressure, hormones, and the like. A sensor may be located in a blood vessel such as the carotid artery, aortic arch, and renal artery. Alternatively, for measurements of heart rate and/or blood pressure, the sensor may be located externally and communicate the information to an external controller or the implantable neuroregulator.

In embodiments, the implantable regulator is configured to respond to information from the sensor to change or modify therapy programs depending on the effect on heart rate, blood pressure, mean arterial pressure, hormones, and combinations thereof at a predetermined level. For example, a blood pressure of greater than about 120/80 mm Hg can result in an activation of the first, second, and/or third therapy program or a blood pressure of about 120/80 mm Hg or less can result in a temporary cessation of the first, second, and/or third therapy program. Likewise, a renin level of greater than about 3 ng/ml/hr when a patient is standing may trigger an activation of the first, second, and/or third therapy program or a renin level of about 3 ng/ml./hr or less can result in a temporary cessation of the first and/or second therapy program. An aldosterone level of greater than about 30 ng/dl when a patient is standing may trigger an activation of the first, second, and/or third therapy program or an aldosterone level of about 30 ng/dl or less can result in a temporary cessation of the first, second, and/or third therapy program. An angiotensin II level of greater than about 0.3 micrograms per deciliter when a patient is standing may trigger a activation of the first, second, and/or third therapy program or angiotensin level of about 0.3 micrograms per deciliter or less can result in a temporary cessation of the first, second and/or third therapy program.

In a specific embodiment, the implantable neuroregulator is configured to activate the first, second and/or third therapy program if blood pressure exceeds a high blood pressure threshold. In embodiments, the high blood pressure threshold is at least 130 mmHg systolic pressure, 90 mm Hg diastolic pressure, or both.

Many factors influence heart rate and blood pressure. Factors include nerve activity, hormones, baroreceptor activity, blood volume, and injury. Nerves associated with cardiac region, renal region, splanchnic region and muscle region influence heart rate and blood pressure. The nerve activity of the renal region includes the first lumbar splanchnic nerve, the renal nerve, nerves of the celiac plexus and the vagus nerve. The nerve activity of the cardiac region includes the vagus nerve at the carotid sinus or aortic arch, sympathetic nerve apparatus innervating the heart, the glossopharyngeal nerve, and baroreceptors. The nerve activity at the splanchnic region includes the first lumber splanchnic nerve, and spinal sympathetic nerves originating from the spinal cord at T10 to L5.

One factor that influences heart rate and blood pressure is sympathetic nerve activity. Sympathetic nerve activity has a baseline level of activity specific to each organ and is adjusted up or down depending on a variety of inputs. The inputs include blood volume, arterial baroreceptors, chemoreceptors, and hormonal levels. Acute changes in blood pressure occur due to acute stress events such as loss of blood, shock, or injury. Sympathetic nerve response is characterized by rapid synchronized bursts of nerve activity as the body is responding to an event that triggers the “fight or flight response”. Chronic changes in blood pressure reflect disease or chronic changes to an organ or blood vessels.

For example, some patients with essential hypertension do not exhibit changes to the heart or kidneys at onset. Other patients develop hypertension or heart failure in conjunction with obesity. Sympathetic nerve activity relating to each organ is altered in different diseases or conditions. In normal weight individuals with hypertension, renal and cardiac sympathetic nerve activities are increased. In obese individuals with hypertension, renal sympathetic nerve activity is increased more than cardiac sympathetic nerve activity. In patients with heart failure, obesity and hypertension the level of sympathetic nerve activation is the highest.

Other factors that influence hypertension include arterial baroreceptor activity, vagal nerve activity, and hormonal factors. Arterial baroreceptor activity is impaired in patients with atherosclerosis or loss of blood vessel flexibility. Hormonal factors include the release of hormones such as leptin, angiotensin, renin, and norepinephrine.

Electrical signal treatments can be applied to alter nerve activity and/or baroreceptor activity in the treatment of conditions associated with an alteration in blood pressure and heart rate. In embodiments, the electrical signal treatment is tailored to address one or more changes to nerve and/or baroreceptor activity in a disease or condition such as heart failure, essential hypertension, obesity related hypertension, sleep apnea, obesity related heart failure, atherosclerosis, chronic kidney disease, metabolic disease, and hypertension or kidney disease associated with diabetes. The electrical signal treatments can also be used in combination with pharmacological agents useful in the treatment of a hypertension, heart failure, chronic renal disease, obesity, and diabetes.

In embodiments, for treatment of hypertension, an electrical signal treatment is applied to downregulate a vagus nerve, downregulate a spinal sympathetic nerve, downregulate a renal nerve, activate a baroreceptor, and combinations thereof. In certain embodiments, an electrical signal treatment is applied intermittently to downregulate activity on a vagus nerve and/or a sympathetic nerve, for example, a spinal sympathetic nerve. In certain embodiments, an electrical signal treatment is applied intermittently to downregulate activity on a vagus nerve and/or a renal nerve or blood vessel. In some cases, the electrical signal treatment is applied in bursts during an ON time followed by an OFF time in order to allow recovery of the nerve. In particular, sympathetic nerve activity is known to occur in coordinated bursts. While not meant to limit the scope of the invention, applying an electrical signal treatment intermittently and/or in bursts may enhance effectiveness for downregulating sympathetic nerve activity and prevent nerve accommodation or resetting. (Malpas, Physiological reviews 90:513-557(2010)). There is some evidence that nerve ablation or continuous stimulation of baroreceptors can lead to resetting of the baroreceptor response or nerve regrowth thereby diminishing the therapeutic response. (Malpas, cited supra).

In some embodiments, the electrical treatment signal is applied to at least partially downregulate the vagus nerve and the electrical signal is selected for frequency, pulse width, amplitude and timing. In some embodiments, the electrical signal is applied intermittently in a cycle including an ON time of application of the signal followed by an OFF time during which the signal is not applied to the nerve, wherein the ON and OFF times are applied multiple times per day over multiple days. In embodiments, the OFF time is selected to allow at least a partial recovery of the nerve.

In some embodiments, the electrical treatment signal is applied to at least partially downregulate a sympathetic nerve and the electrical signal is selected for frequency, pulse width, amplitude and timing. In some embodiments, the electrical signal is applied intermittently in a cycle including an ON time of application of the signal followed by an OFF time during which the signal is not applied to the nerve, wherein the ON and OFF times are applied multiple times per day over multiple days. In embodiments, the OFF time is selected to allow at least a partial recovery of the nerve.

In embodiments, the electrical signal is applied to the renal nerve in a multiplex fashion where one series of pulses is delivered to the renal nerve with a first set of parameters followed by or interleaved with a second set of parameters. In embodiments, the first and second set of parameters only differ in a single parameter such as frequency or pulse amplitude. In a specific embodiment, a first set of pulses has a frequency of about 200 to 10,000 Hz followed by a second set of pulses at a frequency of 1 to 199 Hz. In embodiments, the lead body includes a multitude of electrodes or contacts. When the lead body has a circular cross-sectional shape, the contacts can have a generally ring-type shape and can be spaced apart axially along the length of the lead body. One or more of the contacts are used to provide signals, and another one or more of the contacts provide a signal return path. Accordingly, the lead body delivers monopolar modulation (e.g., if the return contact is spaced apart significantly from the delivery contact), or bipolar modulation (e.g., if the return contact is positioned close to the delivery contact and in particular, at the same target neural population as the delivery contact).

In some embodiments, the electrical treatment signal is applied to at least partially activate baroreceptors and the electrical signal is selected for frequency, pulse width, amplitude and timing. In some embodiments, the signal is applied to a cardiac blood vessel, a glossopharyngeal nerve, or a vagus nerve located at the cardiac notch. In some embodiments, the electrical signal is applied intermittently in a cycle including an ON time of application of the signal followed by an OFF time during which the signal is not applied to the nerve or blood vessel, wherein the ON and OFF times are applied multiple times per day over multiple days. In embodiments, the OFF time is selected to allow at least a partial recovery of the nerve.

An apparatus comprises a device that is programmed to deliver an electrical treatment signal with characteristics of frequency, ON and OFF times, amplitude, location, nerve, selected to provide for control of blood pressure and/or heart rate. In embodiments, some of the parameters of the therapy program are fixed and others are adjustable.

In one aspect, the disclosure provides an apparatus comprising: at least two electrodes; an implantable neuroregulator connected to the electrode and configured to deliver a first therapy program to a first target nerve or blood vessel and a third therapy program to a second target nerve or blood vessel, wherein the first and third therapy programs deliver an electrical signal to each target nerve intermittently with an ON time and an OFF time multiple times in a day. In embodiments, the first therapy program delivers an electrical signal treatment that has a frequency selected to down regulate neural activity on the nerve during an ON time and has an OFF time selected to provide for at least partial recovery of nerve function, and wherein the third therapy program delivers an electrical signal treatment that has a frequency to up regulate neural activity; and an external device, wherein the external device is configured to communicate data and power signals to the neuroregulator and to communicate data to another programming device.

In an embodiment, an apparatus is configured to provide at least one electrode adapted to be place on a nerve or a blood vessel, a neuroregulator connected to the electrode and configured to deliver a therapy program, wherein the therapy program comprises an electrical signal treatment that is applied intermittently with an ON time and an OFF time and has a frequency selected to downregulate activity on the renal nerve, and an external device, wherein the external device is configured to communicate data and power signals to the neuroregulator and to communicate data to another programming device.

The devices of the disclosure can be combined with a drug treatment. In embodiments, the apparatus is configured to store data concerning different drugs useful for the treatment of hypertension or heart failure including the drug or drugs being taken by the patients as well as the dose of drugs.

Another aspect of the disclosure provides a method of treating a condition associated with impaired heart rate and/or blood pressure in a subject comprising: applying an intermittent electrical treatment signal to a target nerve or blood vessel in proximity to the kidney of the subject, wherein said electrical treatment signal is selected to at least partially down-regulate neural activity on the nerve during an ON time and to at least partially restore neural activity on the nerve during an OFF time and wherein the ON and OFF times are applied multiple times per day over multiple days.

In another aspect, the disclosure provides a method of treating hypertension or congestive heart failure comprising: a) selecting a drug for treating hypertension for a patient where effective dosages for treating hypertension for such a patient are associated with disagreeable side effects or inadequate blood pressure control; and b) treating a patient for hypertension with a concurrent treatment comprising: i) applying an intermittent electrical treatment signal to a renal nerve or renal artery of the patient at multiple times per day and over multiple days with the block selected to down-regulate afferent and/or efferent neural activity on the nerve and with neural activity at least partially restoring upon discontinuance of said block; and ii) administering said drug to the patient.

### BRIEF DESCRIPTION OF THE DRAWINGS

FIG. 1 is a schematic representation of an implantable apparatus configuration for applying an electrical signal to a vagus nerve;

FIG. 2 is a schematic representation of an exemplary neuroregulator and leads;

FIG. 3 illustrates a schematic representative of another exemplary embodiment comprising an implantable component comprising a rechargeable neuroregulator 510, connectors (515, e.g. IS-1 connectors) therapy leads (513) and a receiving coil 516. Two leads are connected to the connectors for connection to the implanted circuit. Both have a tip electrode for placement on a nerve.

FIG. 4 shows recovery of the vagal nerve after application of blocking signal;

FIG. 5 shows a typical duty cycle.

FIG. 6 shows effects of electrical signal therapy on excess weight loss for patients in the study of Example 1 as described herein;

FIG. 7A shows the effect of electrical signal therapy on blood pressure for subjects receiving treatment and who did not have elevated blood pressure at the start of treatment and completed 6 months of therapy as described in Example 1. The mean baseline systolic pressure was 115.4 mmHg and the mean baseline diastolic pressure was 68.0 mm Hg. No significant changes were seen in subjects with normal baseline systolic blood pressure (SBP) and diastolic blood pressure (DBP) at 1, 3 or 6 months;

FIG. 7B shows the effect of electrical signal therapy on the change in blood pressure for obese subjects with elevated blood pressure completing 6 months of therapy as described in Example 1. The cohort was defined by elevated systolic pressure of greater than or equal to 140 mmHg or diastolic blood pressure of greater than or equal to 90 mmHg or a history of hypertension. The mean baseline systolic pressure was 141 mmHg and the mean baseline diastolic pressure was 88 mm Hg. Significant changes were seen in subjects with hypertensive baseline systolic blood pressure (SBP) and diastolic blood pressure (DBP) at all time points;

FIG. 7C shows the effect of electrical signal therapy on the change in blood pressure for obese subjects with elevated blood pressure completing 6 months of therapy as described in Example 1. The cohort included patients who had systolic pressure greater than or equal to 140 mmHg and/or diastolic pressure greater than or equal to 90 mmHg and were not diabetic; were diabetic and had systolic pressure of greater than or equal to 130 and/or diastolic pressure greater than 80 mmHg; were diagnosed with hypertension at the time of implantation; or did not have diabetes and had Pre-hypertension with a systolic pressure of 120-139 and/or diastolic pressure of 80-89. The mean baseline systolic pressure was 132.6 mmHg and the mean baseline diastolic pressure was 84.6 mm Hg. Significant changes were seen in subjects with hypertensive baseline systolic blood pressure (SBP) and diastolic blood pressure (DBP) at all time points. The asterisk denotes that the P value is significant for change from baseline +/−SEM.;

FIG. 8 shows the shift in blood pressure in obese patients with and without elevated blood pressure at 6 months of therapy as described in Example 1.

FIG. 9 shows the effect of electrical signal therapy applied to the vagus nerve on the change in mean arterial blood pressure for obese and diabetic subjects in the study as described in Example 2 with elevated blood pressure completing 18 months of therapy. A decrease in mean arterial pressure is seen as early as one week post activation of the device and is maintained for at least 18 month of therapy.

FIG. 10 shows the effect of electrical signal therapy applied to the vagus nerve on the change in diastolic blood pressure for obese and diabetic subjects in the study described in Example 2 completing 18 months of therapy. A decrease in diastolic blood pressure is seen as early as one week post activation of the device and is maintained for at least 18 month of therapy.

FIG. 11 shows the effect of electrical signal therapy applied to the vagus nerve on the change in systolic blood pressure for obese and diabetic subjects in the study described in Example 2 completing 18 months of therapy. A decrease in systolic blood pressure is seen as early as one week post activation of the device and is maintained for at least 18 month of therapy.

FIG. 12A-B show the association of excess weight loss as a function of hours of therapy delivered in the study of Example 3. There was a strong and statistically significant association (repeated measures regression analysis; p<0.001) with improved % EWL from baseline weight with greater hours of device use per day regardless of treatment group. When the device was used for ≧12 h/day, % EWL and % TBWL was 30±4 and 11.4±1.7, respectively in the treated group (n=16) and 22±8 and 8.3±3.0, respectively in the control group (n=14, p=0.42).

FIG. 13A-B show the association of excess weight loss as a function of hours of therapy delivered in the study of Example 3. FIG. 13A shows the excess weight loss as a function of months of treatment for each group based on hours of therapy delivered in the treated group. FIG. 13B shows the excess weight loss as a function of months of treatment for each group based on hours of therapy delivered in the control group.

FIG. 14A-B show the relationship of excess weight loss and decrease in systolic (FIG. 14A) and diastolic (FIG. 14B) blood pressure over a 12 month period in obese subjects receiving 9 hours of therapy or greater.

FIG. 15 shows changes in hypertensive medications in obese subjects receiving 9 hours of therapy or greater.

### DETAILED DESCRIPTION

The following commonly assigned patent and U.S. patent applications are incorporated herein by reference: U.S. Pat. No. 7,167,750 to Knudson et al. issued Jan. 23, 2007; US 2005/0131485 A1 published Jun. 16, 2005, US 2005/0038484 A1 published Feb. 17, 2005, US 2004/0172088 A1 published September 2, 2004, US 2004/0172085 A1 published Sep. 2, 2004, US 2004/0176812 A1 published Sep. 9, 2004 and US 2004/0172086 A1 published Sep. 2, 2004.

This disclosure includes devices and methods for regulating heart rate, blood pressure, and/or chronic kidney disease in a subject. The apparatus provides a reversible, controllable, minimally invasive, safe, and effective way to reduce blood pressure and/or heart rate. In embodiments, a method of treating a condition associated with elevated blood pressure, heart rate, metabolic disease, and/or chronic kidney disease in a subject comprises applying an intermittent neural conduction signal to a target nerve of the subject, with said neural conduction signal selected to modulate neural activity on the nerve and to at least partially restore neural activity on the nerve upon discontinuance of said block.

In some embodiments, the target nerve is the vagus nerve or the renal nerve or both. In embodiments, a first electrode and an additional electrode are placed on the same nerve or different nerves. In some embodiments, the signal is applied below the vagal innervation of the heart. In some embodiments, the electrical signal is selected for frequency, amplitude, pulse width, and timing. The electrical signal may also be further selected to regulate heart rate and/or blood pressure. In some embodiments, the signal is selected for down regulation of neural activity in order to decrease systolic and/or diastolic blood pressure to a predetermined level based on information from a sensor. In other embodiments, a signal may be applied to upregulate neural activity in order to modulate blood pressure. In some embodiments, the parameters of the electrical signal treatment are selected in order to decrease heart rate.

A. Neural Control of Heart Rate and/or Blood Pressure

Hypertension is a cause of heart disease and other related cardiac co-morbidities. Hypertension is a major risk factor for major cardiac events and is associated with mortality due to cardiac events. Hypertension generally relates to high blood pressure, such as a transitory or sustained elevation of systemic arterial blood pressure to a level that is likely to induce cardiovascular damage or other adverse consequences. Hypertension has been defined as a systolic blood pressure equal to or above 140 mmHg and/or a diastolic blood pressure equal to or above 90 mmHg, or for diabetic patients a systolic blood pressure equal to or above 130 mmHg and/or a diastolic blood pressure equal to or above 80 mmHg. Mean arterial pressure (MAP) takes into account pulsatile blood flow in the arteries and is the best measure of perfusion pressure to organs. Pre-hypertension has been defined as a systolic blood pressure of 120 to 139 mmHg and/or a diastolic blood pressure of 80-90 mmHg. (JNC 7, cited supra) When blood vessels constrict, hypertension occurs and the heart works harder to maintain flow at a higher blood pressure. Consequences of uncontrolled hypertension include, but are not limited to, retinal vascular disease, stroke, left ventricular hypertrophy and failure, heart failure, myocardial infarction, dissecting aneurysm, and renovascular disease.

Heart failure occurs when the heart is incapable of maintaining sufficient blood flow to accommodate tissue perfusion and metabolic requirements. Hypertension precedes heart failure in 90% of the cases and increases the risk of heart failure by two to three fold. Drug treatment with some classes of blood pressure medication is useful for controlling disease progression. Controlling blood pressure is one way heart failure is treated. Decreasing systolic blood pressure has been shown to be uniformly beneficial. (JNC 7 at page 35, cited supra).

Other disease conditions in which control of blood pressure and/or heart rate play a role include coronary artery disease, ischemic heart disease, metabolic disease, diabetes, chronic kidney disease, obesity, and cerebrovascular disease. The treatment of these conditions often includes treatment with drugs to lower blood pressure. (JNC 7; cited supra).

The autonomic nervous apparatus (ANS) regulates “involuntary” actions, while the contraction of voluntary (skeletal) muscles is controlled by somatic motor nerves. Examples of organs subject to involuntary actions include respiratory and digestive organs, and also include blood vessels and the heart. Often, the ANS functions in an involuntary, reflexive manner to regulate glands, to regulate muscles in the skin, eye, stomach, intestines and bladder, and to regulate cardiac muscle and the muscle around blood vessels. Both heart rate and blood pressure are controlled via the ANS.

The ANS includes, but is not limited to, the sympathetic nervous apparatus, the enteric nervous apparatus, and the parasympathetic nervous apparatus. The sympathetic nervous apparatus is affiliated with the “fight or flight response” resulting in increases in blood pressure and heart rate to increase skeletal muscle blood flow, and decreases in digestion to provide the energy. The enteric nervous apparatus, sometimes called the second brain, controls the stomach, intestines, and many gastrointestinal functions. The parasympathetic nervous apparatus is affiliated with controlling body functions and decreases blood pressure and heart rate, and increases digestion and manages energy balance.

The cardiovascular (CV) center is located in the medullary center in the brain and controls cardiovascular functions such as heart rate, contractility, and blood vessels. The cardiovascular center receives input from the higher centers in the brain and from afferent fibers of the sympathetic and parasympathetic nerves, including the vagus nerve. The CV center decreases heart rate and can cause vasodilation by parasympathetic activity via efferent impulses carried by the vagus nerve. The CV center can also increase heart rate and cause vasoconstriction via sympathetic stimulation. The major portion of the parasympathetic cranial outflow is via the vagus nerves.

Nerves associated with cardiac region, renal region, splanchnic region and muscle region contribute to the regulation of heart rate and blood pressure. The nerves of the renal region include the first lumbar splanchnic nerve, the renal nerve, nerves of the celiac plexus and the vagus nerve. The nerves of the cardiac region include the vagus nerve at the carotid sinus or aortic arch, sympathetic nerve apparatus innervating the heart, glossopharyngeal nerve, and baroreceptors. The nerves of the splanchnic region include the first lumber splanchnic nerve, and spinal sympathetic nerves originating from the spinal cord at T10 to L5.

One factor affecting heart rate and/or blood pressure is vagal nerve activity. The vagus transmits a diverse array of signals to the central nervous apparatus (CNS) that influence the regulation of cardio- and vaso-motor function and blood pressure, heart rate, neuroimmune modulation, endocrine function as well as gastrointestinal function. For example, the CNS integrates signals from peripheral sites like the liver to modulate blood pressure and glucose. (Bernal-Mizrachi et al, Cell Metabolism 5:91-102, 2007). Infusion of long-chain fatty acids into the portal vein, a major conduit to the liver, has effects that suggest involvement of the CNS including increases in circulating levels of epinephrine and norepinephrine, elevated blood pressure, and accelerated hepatic glucose production (Benthem et al., Am. J. Physio. Endocrin. Metab. 279:E1286-E1293, 2000; Grekin et al., Hypertension 26:193-198, 1995). Hepatic signals are likely transmitted to the CNS by the vagus nerve since vagal activity is increased by portal or jejunal infusion of lipids. The vagus nerve and sympathetic nerves innervate the heart and the blood vessels near the heart. Neural signals from the vagus nerve and other nerves such as the glossopharyngeal nerve, cranial sinus nerve all may influence heart rate and/or blood pressure. In the brainstem, the vagus afferent signals are relayed and influence many of the brainstem cardiovascular control areas that modulate blood pressure and heart rate.

Another factor that influences heart rate and blood pressure is sympathetic nerve activity. Sympathetic nerve activity has a baseline level of activity specific to each organ that can be adjusted up or down depending on a variety of inputs. The inputs include blood volume, arterial baroreceptors, chemoreceptors, and hormonal levels. Acute changes in blood pressure occur due to acute stress events such as loss of blood, shock, or injury. Chronic changes in blood pressure reflect disease or chronic changes to an organ or blood vessels. In response to the inputs, rapid synchronized firing of the sympathetic nerves occur.

For example, some patients with essential hypertension do not exhibit adverse changes to the heart or kidneys at onset. Other patients develop hypertension or heart failure in conjunction with obesity. Sympathetic nerve activity relating to each organ is altered in different disease or conditions. In normal weight individuals with hypertension, renal and cardiac sympathetic nerve activities are increased. In obese individuals with hypertension renal sympathetic nerve activity is increased more than cardiac sympathetic nerve activity. In patients with heart failure, obesity, and hypertension the level of sympathetic nerve activation is the highest.

Other factors that influence hypertension include arterial baroreceptor activity, vagal nerve activity, and hormonal factors. Arterial baroreceptor activity is impaired in patients with atherosclerosis or loss of blood vessel flexibility. Hormonal factors that also influence hypertension include such as leptin, angiotensin, renin, and norepinephrine.

B. Therapy Delivery Apparatus

The disclosure provides devices for regulating blood pressure and/or heart rate comprising a neuroregulator that provides signals to modulate neural activity on a target nerve. The devices and methods are useful, inter alia, in treating hypertension, pre hypertension, congestive heart failure, and hypertension associated with coronary artery disease, ischemic heart disease, chronic kidney disease, obesity, metabolic disease, diabetes, and cerebrovascular disease.

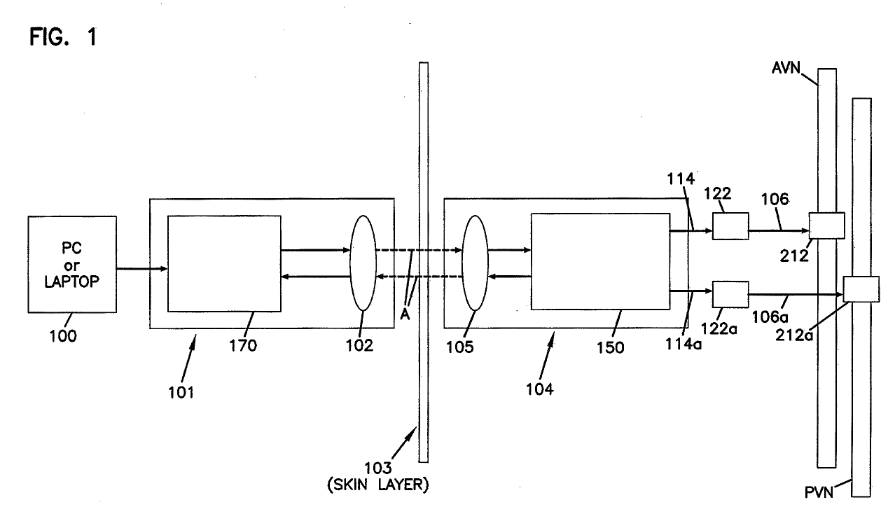

In an embodiment, an apparatus (schematically shown in FIG. 1) for treating such conditions as hypertension, and/or congestive heart failure includes a neuroregulator 104, an external mobile charger 101, and at least one electrode 106. The neuroregulator 104 is adapted for implantation within a patient to be treated. In some embodiments, the neuroregulator 104 is implanted just beneath a skin layer 103. In embodiments, the apparatus includes a sensor for sensing a parameter such as blood pressure, heart rate, oxygen saturation, glucose, cardiac output, lung capacity, hormones, and hematocrit.

i. Electrodes

In some embodiments, referring to FIG. 1, the lead assemblies 106, 106a are electrically connected to the circuitry of the neuroregulator 104 by conductors 114, 114a. Each lead includes at least one electrode. Industry standard connectors 122, 122a are provided for connecting the lead assemblies 106, 106a to the conductors 114, 114a. As a result, leads 116, 116a and the neuroregulator 104 may be separately implanted. Also, following implantation, lead 116, 116a may be left in place while the originally placed neuroregulator 104 is replaced by a different neuroregulator.

The lead assemblies 106, 106a provide electrical signals that up-regulate and/or down-regulate nerves of a patient based on the therapy signals provided by the neuroregulator also referred to as a neuroregulator 104. In an embodiment, the lead assemblies 106, 106a include distal electrodes 212, 212a, which are placed on one or more nerves of a patient. In embodiments, the lead body includes a multitude of electrodes or contacts. When the lead body has a circular cross-sectional shape, the contacts can have a generally ring-type shape and can be spaced apart axially along the length of the lead body. For example, electrodes 212, 212a may be individually placed on the vagal trunks of a patient. For example, the leads 106, 106a have distal electrodes 212, 212a which are individually placed on the anterior and posterior vagal nerves AVN, PVN, respectively, of a patient, for example, just below the patient's diaphragm. Fewer or more electrodes can be placed on or near fewer or more nerves. In some embodiments, the electrodes are cuff electrodes.

At least one electrode is adapted to deliver electrical signal treatments by placement on a nerve or blood vessel. In embodiments, when electrical signal treatment is being applied to arterial baroreceptors or on a complex of nerve and blood vessels such as the renal nerve or celiac plexus, it may be preferable to place the electrode on a blood vessel. Electrodes adapted for placement on a blood vessel may be intravascular or extravascular. In embodiments, electrodes adapted for placement on a blood vessel intravascularly include attachment structures to maintain the electrode in place in the vicinity of the nerve. In embodiments, electrodes applied external to a blood vessel are adapted to the size of the blood vessel as some blood vessels are much larger than others. In other embodiments, an electrode is adapted for placement on a nerve such as a vagus nerve or a splanchnic nerve.

In embodiments, a first electrode is adapted to be placed on a first target nerve or blood vessel selected from the group consisting of renal artery, renal nerve, celiac plexus, a splanchnic nerve, cardiac sympathetic nerves, and spinal nerves originating between T10 to L5 and at least one additional electrode adapted to be placed on a second target nerve or blood vessel selected from the group consisting of vagus nerve, renal artery, renal nerve, celiac plexus, a splanchnic nerve, cardiac sympathetic nerves, spinal nerves originating between T10 to L5, glossopharyngeal nerve, and tissue containing baroreceptors. In embodiments, the first and additional electrodes are each placed on the same nerve or on different nerves.

In other embodiments, an electrode can be placed on the vagus nerve on a location near the SA node of the heart, the carotid sinus or the aortic arch. Electrodes may also be placed intravascularly in the ascending aorta or carotid arteries. In other embodiments, an electrode may be placed on the vagus nerve at a supradiaphragmatic location. In some embodiments, an electrode may be placed on the vagus nerve at a subdiaphragmatic location and an additional electrode placed on the right vagus nerve near the SA node of the heart, in the tissue surrounding the glossopharyngeal nerve or cardiac sinus nerve, or on tissue containing baroreceptors. In embodiments an electrode is adapted to be placed on a vagus nerve and an additional electrode is adapted to be placed on a cardiac sympathetic nerve, a spinal sympathetic nerve, or a splanchnic nerve. In embodiments, any combination of electrode placements can be utilized in the methods of the disclosure.

In other embodiments, a first electrode can be placed on the sympathetic nerve such as first lumbar splanchnic nerve, sympathetic nerves innervating the heart, the renal nerve, and sympathetic nerves originating from the spinal cord at T10 to L5. Electrodes may also be placed intravascularly in the renal artery. In some embodiments, an electrode may be placed on the vagus nerve at a subdiaphragmatic location and another electrode placed on the renal nerve or first lumbar splanchnic nerve. In other embodiments, an electrode may be placed on the vagus nerve at a cardiac sinus or aortic arch region and another electrode placed on the renal nerve or first lumbar splanchnic nerve. In embodiments, any combination of electrode placements can be utilized in the methods of the disclosure.

In another embodiment, an additional electrode is adapted to be placed on a glossopharyngeal nerve, and/or tissue containing baroreceptors. For placement on tissue containing baroreceptors, an electrode may be placed intravascularly or extravascularly. In embodiments, the electrode is placed within or on the aortic arch or within the carotid artery.

The electrical connection of the electrodes to a neuroregulator may be as previously described in FIG. 1 by having a lead (e.g. 106,106a) connecting the electrodes directly to an implantable neuroregulator (eg. 104). Alternatively, and as previously described, electrodes may be connected to an implanted antenna for receiving a signal to energize the electrodes.

While any of the foregoing electrodes could be flat metal pads (e.g., platinum), the electrodes can be configured for various purposes. In an embodiment, an electrode is carried on a patch. In other embodiments, the electrode is segmented into two portions both connected to a common lead and both connected to a common patch. In some embodiments, each electrode is connected to a lead and placed to deliver a therapy from one electrode to another. A flexible patch permits articulation of the portions of the electrodes to relieve stresses on the nerve. In embodiments, for delivering a multiplexed electrical signal a lead comprises an array of electrodes. When the lead body has a circular cross-sectional shape, the contacts can have a generally ring-type shape and can be spaced apart axially along the length of the lead body.

ii. External Charger

The external mobile charger 101 includes circuitry for communicating with the implanted neuroregulator (neuroregulator) 104. In some embodiments, the communication is a two-way radiofrequency (RF) signal path across the skin 103 as indicated by arrows A. Example communication signals transmitted between the external charger 101 and the neuroregulator 104 include treatment instructions, patient data, and other signals as will be described herein. Energy or power also can be transmitted from the external charger 101 to the neuroregulator 104 as will be described herein.

In the example shown, the external charger 101 can communicate with the implanted neuroregulator 104 via bidirectional telemetry (e.g. via radiofrequency (RF) signals). The external charger 101 shown in FIG. 1 includes a coil 102, which can send and receive RF signals. A similar coil 105 can be implanted within the patient and coupled to the neuroregulator 104. In an embodiment, the coil 105 is integral with the neuroregulator 104. The coil 105 serves to receive and transmit signals from and to the coil 102 of the external charger 101.

For example, the external charger 101 can encode the information as a bit stream by amplitude modulating or frequency modulating an RF carrier wave. The signals transmitted between the coils 102, 105 preferably have a carrier frequency of about 6.78 MHz. For example, during an information communication phase, the value of a parameter can be transmitted by toggling a rectification level between half-wave rectification and no rectification. In other embodiments, however, higher or lower carrier wave frequencies may be used.

In an embodiment, the neuroregulator 104 communicates with the external charger 101 using load shifting (e.g., modification of the load induced on the external charger 101). This change in the load can be sensed by the inductively coupled external charger 101. In other embodiments, however, the neuroregulator 104 and external charger 101 can communicate using other types of signals.

In an embodiment, the neuroregulator 104 receives power to generate the therapy signals from an implantable power source 151 such as a battery. In a preferred embodiment, the neuroregulator further comprises a power source, wherein the power source 151 is a rechargeable battery. In some embodiments, the power source 151 can provide power to the implanted neuroregulator 104 when the external charger 101 is not connected. In other embodiments, the external charger 101 also can be configured to provide for periodic recharging of the internal power source 151 of the neuroregulator 104. In an alternative embodiment, however, the neuroregulator 104 can entirely depend upon power received from an external source. For example, the external charger 101 can transmit power to the neuroregulator 104 via the RF link (e.g., between coils 102, 105).

In some embodiments, the neuroregulator 104 initiates the generation and transmission of therapy signals to the lead assemblies 106, 106a. In an embodiment, the neuroregulator 104 initiates therapy when powered by the internal battery 151. In other embodiments, however, the external charger 101 triggers the neuroregulator 104 to begin generating therapy signals. After receiving initiation signals from the external charger 101, the neuroregulator 104 generates the therapy signals (e.g., pacing signals) and transmits the therapy signals to the lead assemblies 106, 106a.

In other embodiments, the external charger 101 also can provide the instructions according to which the therapy signals are generated (e.g., pulse-width, amplitude, and other such parameters). In a preferred embodiment, the external charger 101 includes memory in which several programs/therapy schedules can be stored for transmission to the neuroregulator 104. The external charger 101 also can enable a user to select a program/therapy schedule stored in memory for transmission to the neuroregulator 104. In another embodiment, the external charger 101 can provide treatment instructions with each initiation signal.

Typically, each of the programs/therapy schedules stored on the external charger 101 can be adjusted by a physician to suit the individual needs of the patient. For example, a computing device (e.g., a notebook computer, a personal computer, tablet computer, etc.) 100 can be communicatively connected to the external charger 101. With such a connection established, a physician can use the computing device 100 to program therapies or individual parameters for a therapy program into the external charger 101 for either storage or transmission to the neuroregulator 104. In embodiments, the computing device is a clinician programmer that is dedicated to transmitting instructions for parameters for therapy programs, receiving and storing information from the external mobile charger and/or neuroregulator, clinical information for each patient such as drugs and dosages, and generating reports for one or more patients with implanted therapy devices as described herein.

Referring to FIG. 1, the circuitry 170 of the external mobile charger 101 can be connected to an external coil 102. The coil 102 communicates with a similar coil 105 implanted within the patient and connected to the circuitry 150 of the neuroregulator 104. Communication between the external mobile charger 101 and the neuroregulator 104 includes transmission of pacing parameters and other signals as will be described.

Having been programmed by signals from the external mobile charger 101, the neuroregulator 104 generates upregulating signals or downregulating signals to the leads 106, 106a. As will be described, the external mobile charger 101 may have additional functions in that it may provide for periodic recharging of batteries within the neuroregulator 104, and also allow record keeping and monitoring.

While an implantable (rechargeable) power source for the neuroregulator 104 is preferred, an alternative design could utilize an external source of power, the power being transmitted to an implanted module via the RF link (i.e., between coils 102, 105). In this alternative configuration, while powered externally, the source of the specific blocking signals could originate either in the external power source unit, or in the implanted module.

The electronic energization package may, if desired, be primarily external to the body. An RF power device can provide the necessary energy level. The implanted components could be limited to the lead/electrode assembly, a coil and a DC rectifier. With such an arrangement, pulses programmed with the desired parameters are transmitted through the skin with an RF carrier, and the signal is thereafter rectified to regenerate a pulsed signal for application as the stimulus to the vagus nerve to modulate vagal activity. This would virtually eliminate the need for battery changes.

However, the external transmitter must be carried on the person of the patient, which is inconvenient. Also, detection is more difficult with a simple rectification apparatus, and greater power is required for activation than if the apparatus were totally implanted. In any event, a totally implanted apparatus is expected to exhibit a relatively long service lifetime, amounting potentially to several years, because of the relatively small power requirements for most treatment applications. Also, as noted earlier herein, it is possible, although considerably less desirable, to employ an external neuroregulator with leads extending percutaneously to the implanted nerve electrode set. The major problem encountered with the latter technique is the potential for infection. Its advantage is that the patient can undergo a relatively simple procedure to allow short term tests to determine whether the condition associated with excess weight of this particular patient is amenable to successful treatment. If it is, a more permanent implant may be provided.

According to an embodiment of the present invention, an apparatus is disclosed for applying an electrical signal to an internal anatomical feature of a patient. The apparatus includes at least one electrode for implantation within the patient and placement at the anatomical feature (e.g., a nerve) for applying the signal to the feature upon application of the signal to the electrode. An implantable component is placed in the patient's body beneath a skin layer and having an implanted circuit connected to the electrode. The implanted circuit includes an implanted communication antenna. An external component has an external circuit with an external communication antenna for placement above the skin and adapted to be electrically coupled to the implanted antenna across the skin through radiofrequency transmission. The external circuit has a plurality of user interfaces including an information interface for providing information to a user and an input interface for receiving inputs from the user.

iii. Neuroregulator

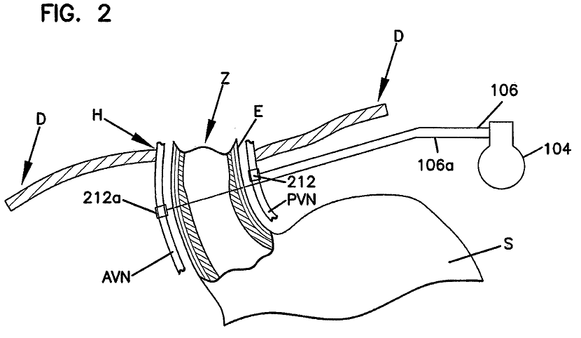

With reference to FIG. 2, an exemplary device is shown for application of a signal to a nerve. The vagus nerve is provided for illustrative purposes only and other nerves may similarly be contacted with a device as described herein. For example, a stomach S is shown schematically for the purpose of facilitating an understanding of applying a vagal nerve modulating signal. The esophagus E passes through the diaphragm D at an opening or hiatus H. In the region where the esophagus E passes through the diaphragm D, trunks of the vagal nerve (illustrated as the anterior vagus nerve AVN and posterior vagus nerve PVN) are disposed on opposite sides of the esophagus E. It will be appreciated that the precise location of the anterior and posterior vagus nerves AVN, PVN relative to one another and to the esophagus E are subject to a wide degree of variation within a patient population. However, for most patients, the anterior and posterior vagus nerves AVN, PVN are in close proximity to the esophagus E at the hiatus H where the esophagus E passes through the diaphragm D.

The anterior and posterior vagus nerves AVN, PVN divide into a plurality of trunks that innervate the stomach directly and via the enteric nervous apparatus and may include portions of the nerves which may proceed to other organs such as the pancreas, kidney, gallbladder and intestines. Commonly, the anterior and posterior vagus nerves AVN, PVN are still in close proximity to the esophagus E and stomach (and not yet extensively branched out) at the region of the junction of the esophagus E and stomach S. In the region of the hiatus H, there is a transition from esophageal tissue to gastric tissue. This region is referred to as the Z-line (labeled “Z” in the Figure). Above the Z-line, the tissue of the esophagus lacks a serosa. Below the Z-line, the tissue of the esophagus E and stomach S are substantially thickened and more vascular. Within a patient population, the Z-line is in the general region of the lower esophageal sphincter. This location may be slightly above, slightly below or at the location of the hiatus H. The electrode is adapted for placement on a vagus nerve or the celiac plexus below the diaphragm of the patient.

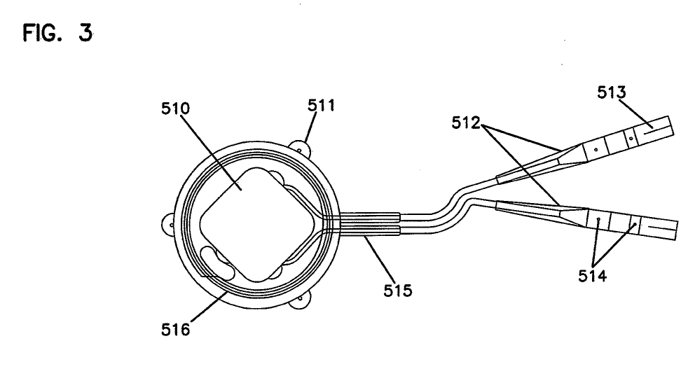

Another embodiment of a device useful in treating a condition associated with impaired blood pressure regulation as described herein is shown in FIG. 3. With reference to FIG. 3, a device comprises an implantable device comprising a rechargeable neuroregulator (5101) that produces electrical pulses that are delivered to the nerve or blood vessels through electrically conductive leads. In addition to delivering electrical pulses, the rechargeable neuroregulator also receives command signals from the clinician programmer (not shown) and uploads data to the programmer via the external charger (not shown). The rechargeable neuroregulator is powered by an internal rechargeable battery. The internal battery is periodically recharged by RF power that is radiated by the transmit coil (not shown)and picked up by a receiving antenna (516) on the rechargeable neuroregulator. Two bipolar leads connect the rechargeable neuroregulator to the nerve (512). In this embodiment, each lead has two electrodes (513). In embodiments, one electrode is positioned around the nerve trunk and the other is in electrical contact with nearby tissue. The external charger (not shown) provides the electrical excitation of the transmit coil needed to deliver RF power to the rechargeable neuroregulator. In addition, it serves as an interface for communications (not shown) between the rechargeable neuroregulator (510) and the clinician programmer(not shown). In embodiments, a rechargeable battery is used to power the external charger.

In an embodiment, the nerves are indirectly stimulated by passing electrical signals through the tissue surrounding the nerves. In some embodiments, the electrodes are bipolar pairs (i.e. alternating anode and cathode electrodes). In some embodiments, a plurality of electrodes may be placed overlying the anterior and/or posterior vagus nerves AVN, PVN. As a result, energizing the plurality of electrodes will result in application of a signal to the anterior and posterior vagus nerves AVN, PVN and/or their branches. In some therapeutic applications, some of the electrodes may be connected to a blocking electrical signal source (with a blocking frequency and other parameters as described below). Of course, only a single array of electrodes could be used with all electrodes connected to a blocking or a downregulating signal.

The neuroregulator generates electrical signals in the form of electrical impulses according to a programmed regimen. In embodiments, the therapy programs include a first therapy program having parameters that provide for at least partial down regulation of a first target nerve, a second therapy program having parameters that provide for at least partial down regulation of a second target nerve, and a third therapy program having parameters that provide for at least partial up regulation of a first or second target nerve. In each program, each of the individual parameters may be fixed or adjustable. Combinations of therapy programs may be applied to the same nerves or different nerves. Combinations of therapy programs can be delivered during the same on time or different on times. For example, a first therapy program for downregulation of a first target nerve and a second therapy for downregulation of a second target nerve may be applied at the same time. In another example, a second therapy program is applied to down regulate a vagus nerve or renal nerve and a third therapy program is applied to upregulate a glossopharyngeal nerve and/or baroreceptors at the same time.

The neuroregulator utilizes a microprocessor and other electrical and electronic components, and communicates with an external programmer and/or monitor by asynchronous serial communication for controlling or indicating states of the device. Passwords, handshakes and parity checks are employed for data integrity. The neuroregulator also includes means for conserving energy, which is important in any battery operated device and especially so where the device is implanted for medical treatment of a disorder, and means for providing various safety functions such as preventing accidental reset of the device.

Features may be incorporated into the neuroregulator for purposes of the safety and comfort of the patient. In some embodiments, the patient's comfort would be enhanced by ramping the application of the signal up. The device may also have a clamping circuit to limit the maximum voltage (20 volts for example) deliverable to the vagus nerve, to prevent nerve damage. An additional safety function may be provided by implementing the device to cease signal application in response to manual deactivation through techniques and means similar to those described above. In this way, the patient may interrupt the signal application if for any reason it suddenly becomes intolerable.

In embodiments, one or more neuroregulators are employed, to provide upregulation or downregulation to a nerve or blood vessel. Use of implanted neuroregulators for performing the method of the invention is preferred, but treatment may conceivably be administered using external equipment on an outpatient basis, albeit only somewhat less confining than complete hospitalization. Implantation of one or more neuroregulators, of course, allows the patient to be completely ambulatory, so that normal daily routine activities including on the job performance is unaffected.

The neuroregulator 104 also may include memory in which treatment instructions and/or patient data can be stored. For example, the neuroregulator 104 can store one or more therapy programs indicating what therapy should be delivered to the patient. The neuroregulator 104 also can store patient data indicating how the patient utilized the therapy apparatus and/or reacted to the delivered therapy. The neuroregulator can also store data relating to any sensed parameters that can then be accessed by the health care provider. For example, if blood pressure is stable for a period of time, the health care provider may choose to program the neuroregulator for maintenance mode.

The implantable neuroregulator is configured to deliver a first therapy program, a second therapy program and/or a third therapy program. In embodiments, a first therapy program delivers an electrical signal treatment to the first target nerve or blood vessel intermittently with an on time and an off time multiple times in a day, wherein the first therapy program delivers an electrical signal treatment that has a frequency selected to down regulate neural activity on the first nerve or blood vessel during an on time and has an off time selected to provide for at least partial recovery of nerve function. In embodiments, a second therapy program delivers an electrical signal to second target nerve or blood vessel intermittently with an on time and an off time multiple times in a day, wherein the second therapy program delivers an electrical signal treatment that has a frequency to down regulate neural activity. The first and second therapy program can differ in one or more parameters but both have electrical signal parameters that provide for a downregulation of nerve activity. In some embodiments, the first therapy program is applied to both the first and second target nerve. In some embodiments, the second therapy program is applied to the first and second target nerve. In embodiments, a third therapy program delivers an electrical signal to first and/or second target nerve or blood vessel intermittently with an on time and an off time multiple times in a day, wherein the third therapy program delivers an electrical signal treatment that has a frequency to up regulate neural activity. Other parameters of the electrical signal treatment are selectable by a user such as frequency, pulse width, amplitude, voltage, on time, off time, and the like.

The neuroregulator can maintain and store a number of parameters relating to therapy programs. In embodiments, such parameters include frequency, amplitude, pulse width, on time, off time, ramp up time, ramp down time, and the like. In some embodiments, some of the parameter values are fixed and others are programmable by the health care provider to tailor the therapy for the condition and efficacy. One or more of the parameters can be selected so as to constitute an adjustable therapy program for a particular application. In embodiments, a first, second and a third therapy program are stored in the neuroregulator. In some cases, each therapy program is adjustable.

For example, parameters are stored for an electrical signal treatment that provides a downregulating signal to a vagus nerve. In other embodiments, electrical signal treatment parameters are stored for downregulation of neural activity on a sympathetic nerve or on a renal nerve. In embodiments a therapy program for downregulation of a renal nerve involves multiplexing the electrical signal treatment where one series of pulses are delivered to the renal nerve with a first set of parameters followed by or interleaved with a second set of parameters. In yet other embodiments, electrical signal parameters are stored for upregulating a baroreceptor. One or more of the therapy programs can be stored on the neuroregulator or on the external charger or both.

The intermittent aspect of the electrical signal treatment resides in applying the signal according to a prescribed duty cycle. The pulse signal is programmed to have a predetermined on-time in which a train or series of electrical pulses of preset parameters is applied to the nerve or blood vessel, followed by a predetermined off-time. Nevertheless, continuous application of the electrical pulse signal may also be effective.

In some embodiments, signals can also be applied at a portion of the nervous apparatus remote from the vagus nerve at the subdiaphragmatic location such as at or near the cardiac notch. Signals can also be applied at other sympathetic nerves and/or baroreceptors in combination with application of a signal to the vagus nerve such as a down regulating signal. Here, at least one neuroregulator is implanted together with one or more electrodes operatively coupled to the neuroregulator via leads for generating and applying the electrical signal internally to a portion of the patient's nervous apparatus to provide indirect blocking, down regulation, or up regulation of the vagus nerve or other nerves or receptors in the vicinity of the desired location. Different therapy programs are stored on the neuroregulator to deliver an electrical signal treatment tailored to the patient's condition and efficacy of the treatment.

In some embodiments, the electrical signal is applied intermittently to downregulate the vagus nerve at the cardiac region without application of any other downregulating and/or upregulating signal on the vagus nerve or other nerves.

It is surprising that downregulation of the vagus nerve at a location distal to the innervation of the cardiac region, e.g. subdiaphragmatically, would be effective to decrease blood pressure and heart rate. In some cases, the blood pressure is decreased to or near the normal range. In a typical situation of high blood pressure, the vagus nerve operates to slow the heart rate to assist in decreasing the blood pressure and thus, it is surprising that downregulating and/or blocking of the vagus nerve would be effective to lower heart rate and blood pressure. In addition, clinical benefits may include lowering the blood pressure early in treatment and with minimal adverse clinical effects. Little or no side effects have been observed with this treatment in contrast to side effects often associated with drug treatment. Patients without hypertension or without prehypertension show no effect on blood pressure during electrical signal treatment. Combining the downregulating electrical signal treatment of the vagus nerve at a subdiaphragmatic location with that of downregulating a second target nerve or upregulating a second target nerve provides additional efficacy in controlling blood pressure.

In some embodiments, the electrical signal is applied intermittently to downregulate a renal nerve without application of any other downregulating and/or upregulating signal on the vagus nerve or other nerves. In embodiments, the electrical signal treatment of the renal nerve is combined with administration of a pharmacological agent and/or the result of a sensed increase in blood pressure and/or heart rate.

Alternatively, the electrical signal may be applied non-invasively to a blood vessel for indirect application of electrical signal treatment. The electrical signal may be applied to an electrode positioned intravascularly to provide, for example, an upregulating signal to a baroreceptor or a down regulating signal to a renal nerve.

A number of different parameters associated with a therapeutic program are stored on the neuroregulator to allow the physician to select a combination of electrical signal treatment that may be of benefit to the patient depending on the conditions exhibited by the patient and/or modified as a result of treatment efficacy. In embodiments, to decrease heart rate and/or blood pressure, a therapy program provides an electrical signal treatment based on the target nerve and disease or disorder for the patient. In yet other embodiments, the health care provider is able to select from a number of therapy program options depending on the patient's condition and the target nerve as described below.

In embodiments, the implantable neuroregulator is configured to operate in multiple modes. In embodiments, the modes include a first mode, a second mode, and a maintenance mode. In embodiments, a first mode comprises providing the first therapy program to the first electrode and the second therapy program to the additional electrode, wherein the first therapy and second therapy program deliver an electrical signal that down regulates activity on the first and second target nerves, and a second mode comprises providing the first therapy program to the first electrode and the third therapy program to the additional electrode, wherein the third therapy program delivers an electrical signal treatment that upregulates activity on the target nerve.

In embodiments, a maintenance mode is one in which the neuroregulator delivers low energy electrical signals associated with safety checks and impedance checks for a period of time of 9 hours or less. In the interest of conserving battery power, the device may remain on but deliver the safety and impedance checks for 30 minutes to 9 hours, 1 hour to 8 hours, 1 hour to 7 hours, 1 hour to 6 hours, 1 hour to 5 hours, 1 hour to 4 hours, 1 hour to 3 hours and 1 hour to 2 hours. In embodiments, the safety checks are delivered at 50 Hz or less at least every 0.2 μs and impedance checks are delivered once every two minutes at a frequency of 1000 Hz or more. While not meant to limit the scope of the invention, it is believed that a therapeutic effect is associated with this low energy electrical single treatment if applied for at least 9 hours per day and not at shorter time periods. If the patient condition has stabilized or resolved, a health care provider may program the device for maintenance mode, leaving open the option to initiate a therapy program once again at a later date.

In embodiments, the neuroregulator also collects and transmits information on the effectiveness of the dose and timing of administration of anti-hypertensive medications. For example, a patient may start at a lower dose of a medication that recommended, especially to avoid side effects, in conjunction with an electrical signal treatment and have dosage increased only if adequate blood pressure control is not achieved. In addition, the patient may try taking the medication at different times of the day to determine whether the efficacy of the medication is increased.

The neuroregulator may be programmed with a programming wand and a personal computer using suitable programming software developed according to the programming needs and signal parameters which have been described herein. The intention, of course, is to permit noninvasive communication with the electronics package after the latter is implanted, for both monitoring and programming functions. Beyond the essential functions, the programming software should be structured to provide straightforward, menu-driven operation, HELP functions, prompts, and messages to facilitate simple and rapid programming while keeping the user fully informed of everything occurring at each step of a sequence. Programming capabilities should include capability to modify the electronics package's adjustable parameters, to test device diagnostics, and to store and retrieve telemetered data. It is desirable that when the implanted unit is interrogated, the present state of the adjustable parameters is displayed on the PC monitor so that the programmer may then conveniently change any or all of those parameters at the same time; and, if a particular parameter is selected for change, all permissible values for that parameter are displayed so that the programmer may select an appropriate desired value for entry into the neuroregulator.

In embodiments, adjustable parameters include frequency, pulse width, on and off times, current, and ON/OFF ramps. One or more of the parameters are selected to decrease heart rate and/or blood pressure without adverse clinical effects. In embodiments, the adjustable parameters are current amplitude, on times and off times, and ramp times.

A first therapy and/or a second therapy program delivers an electrical signal treatment that downregulates activity on the nerve. The frequency is selected to provide at least a partial decrease in activity of the first and/or second target nerve. In some embodiments, the neuroregulator is configured to deliver a signal of about 200 Hz to 25 kHz, 200 Hz to about 15 kHz, 200 Hz to about 10 kHz, 200 to 5000 Hz, 250 to 5000 Hz, 300 to 5000 Hz, 400 to 5000 Hz, 500 to 5000 Hz, 200 to 2500 Hz, 300 to 2500 Hz, 400 to 2500 Hz, 500 to 2500 Hz, and any frequencies in between 200 Hz to 25 kHz or combinations thereof.

In embodiments, nerve activity can be blocked using low frequency baseline modulation. For example, in the initial negative portion of a biphasic pulse, the amplitude is increased (or could be decreased) by (for example) 100 μA, producing a direct current offset which could be effective in achieving a neural block. In the subsequent positive portion of the biphasic pulse, a compensatory amplitude is increased by the same 100 μA, also producing a direct current offset which could be effective in achieving a neural block, and ensuring that the net current/charge transmitted to the tissue during one biphasic pulse cycle, is zero. In other embodiments, increased (or decreased) pulse widths in the negative and positive regions of the biphasic pulse achieves the same effect of direct current/charge offset, while maintaining the net charge per biphasic pulse cycle, at zero.

A third therapy program delivers an electrical signal treatment that up regulates activity on the nerve. In embodiments, a frequency is selected to provide at least a partial increase in activity of the nerve such as a glossopharyngeal nerve or baroreceptors. In some embodiments, the neuroregulator is configured to deliver a signal of about 0 to 200 Hz, 1 to 175 Hz, 1 to 150 Hz, 1 to 125 Hz, 1 to 100 Hz, 1 to 75 Hz, 1 to 50 Hz, 1 to 25 Hz, 1 to 10 Hz, and any frequencies in between 1 to 200 Hz or combinations thereof. A net current/charge of 0 is achieved using low frequency baseline modulation as described above.

While the disclosure contemplates that different therapy programs will be applied to different target nerves or blood vessels, different therapy programs can be employed on the same nerve or blood vessel at different locations. A combination of low frequency and high frequency signals may also applied to a single nerve type. For example a down regulating signal may be applied at a vagus nerve below a vagal innervation of the heart and an upregulating signal can be employed at the vagus nerve at the carotid artery or aortic arch. Another example, involves maintenance mode which employs a high frequency signal for an impedance check in combination with a low frequency signal for safety checks. The down regulating and upregulating signals can be applied during the same on time or on different times.

In embodiments, when sympathetic nerve activity is modulated, the timing and frequency of the electrical signal treatment is modified in order to at least partially block rapid synchronized bursts of nerve activity. In embodiments, the electrical signal is applied to the renal nerve in a multiplex fashion where one series of pulses are delivered to the renal nerve with a first set of parameters followed by or interleaved with a second set of parameters. In embodiments, the first and second set of parameters only differ in a single parameter such as frequency or pulse amplitude. In a specific embodiment, a first set of pulses has a frequency of about 200 to 10,000 Hz followed by a second set of pulses at a frequency of 1 to 199 Hz. In embodiments, the current amplitude of the signal is about 0.5 to 18 mA, but preferably at least 6 mA.

The ON times are selected to provide at least a partial decrease or increase in nerve activity. In embodiments, the neuroregulator is configured to deliver ON times of from 30 seconds to 30 minutes, 30 seconds to 20 minutes, 30 seconds to 10 minutes, 30 seconds to 5 minutes, 30 sec to 3 minutes, 30 seconds to 2 minutes, or 30 seconds to 1 minute or combinations thereof. The OFF times are selected to allow at least partial recovery of the nerve activity. In embodiments, the neuroregulator is configured to deliver OFF times of from 30 seconds to 30 minutes, 30 seconds to 20 minutes, 30 seconds to 10 minutes, 30 seconds to 5 minutes, 30 sec to 3 minutes, 30 seconds to 2 minutes, or 30 seconds to 1 minute or combinations thereof.

In other embodiments other on times and off time may be utilized as appropriate for the patient's condition and responsiveness to treatment. For example, the ON times may be 30 minutes or longer followed by an OFF time of at least 24 hours or longer. A specific embodiment includes one or more therapy on periods of up to 30 minutes with intervening therapy off periods for up to 7 days or longer.

In embodiments, the current and/or voltage are adjusted based on safety and efficacy of treatment for the patient. In some embodiments, the signal amplitude can range from 0.5 mA to about 18 mA including amplitudes in between that differ by 0.25 mA or other larger or smaller increments, adjusted up or down based on patient response. Voltages can range from 0.25 volts up to 20 volts or voltage in between that differ by 0.25 volts, or other larger or smaller increments, adjusted up or down based on patient response. In embodiments, current amplitude is about 0.5 to 14, 0.5 to 12, 0.5 to 10, 0.5 to 8, 0.5 to 6, 0.5 to 4, 0.5 to 2, and 0.5 to mA.

The treatment time can be at least 9 hours, an entire 24 hour period, 18 to 24 hours, 16 to 24 hours, 12 to 24 hours, and 8 to 24 hours, 6 to 24 hours, 4 to 24 hours or other intervals that match the treatment needs and/or activities of daily living of the patient or combinations thereof. Treatment time may be varied depending on whether the patient experiences a drop in blood pressure while sleeping. (Pickering et al, N. Eng. J. Med. 354:22 (2002)). Some patients who have hypertension have a blood pressure of greater than or equal to 135/85 mm Hg while they are awake and less than or equal to 120/75 mm Hg when they are asleep. For those patients, the treatment would not be administered during some of the sleeping hours of the patient. However, in most cases, treatment would resume as early as 4 am in order to minimize the early morning spike in blood pressure that can lead to heart attack or stroke. (Pickering et al, cited supra) In other cases, for those patients who do not experience a drop in blood pressure while they are sleeping, treatment may be administered for a full 24 hour period.

Other desirable features of appropriate software and related electronics would include the capability to store and retrieve historical data, including patient code, device serial number, number of hours of battery operation, number of hours of output, sensed parameters, and number of magnetic activations (indicating patient intercession) for display on a screen with information showing date and time of the last one or more activations.

Diagnostic testing should be implemented to verify proper operation of the device, and to indicate the existence of problems such as with communication, the battery, or the lead/electrode impedance. A low battery reading, for example, would be indicative of imminent end of life of the battery and need for implantation of a new device. However, battery life should considerably exceed that of other implantable medical devices, such as cardiac pacemakers, because of the relatively less frequent need for activation of the pulse generator of the present invention. In any event, the nerve electrodes are capable of indefinite use absent indication of a problem with them observed on the diagnostics testing.

The device may utilize circadian or other programming as well, relating to the historical increase in blood pressure or heart rate experienced by the patient including early morning spikes in blood pressure and/or spikes in heart rate and/or blood pressure due to sleep apnea.

The neuroregulator may also be activated manually by the patient by any of various means by appropriate implementation of the device. These techniques include the patient's use of an external magnet, or of an external RF signal generator, or tapping on the surface overlying the neuroregulator, to activate the neuroregulator and thereby cause the application of the desired modulating signal to the electrodes. Another form of treatment may be implemented by programming the neuroregulator to periodically deliver the vagal activity modulation productive of glycemic control at programmed intervals.

iv. Sensor

In embodiments, the apparatus includes a sensor for patient status. In embodiments, the sensor measures, for example, heart rate, blood pressure, blood oxygen saturation levels, sleep apnea events, lung capacity, hematocrit, cardiac output, blood glucose, and combinations thereof. The sensor can be integrated into the electrode or separately positioned in order to measure the patient status with respect to one or more parameters. An implantable sensor is operatively coupled to the implantable neuroregulator through a lead. The sensor can be located externally and provide information to the implantable device and/or health care provider by a wireless communication through a mobile device.

An increase in blood pressure, heart rate above a predetermined level, and/or a decrease in blood oxygen saturation levels below a predetermined level will trigger selection of electrical signal treatment to adjust blood pressure, heart rate, and oxygen levels back to a predetermined level. In embodiments, a predetermined level for blood pressure includes a systolic pressure of 130 mmHg or greater and a diastolic pressure of 80 mmHg or greater. For heart rate, a predetermined level includes 85 beats per minute or greater. For blood oxygen saturation levels, a predetermined level includes 94% oxygen saturation or less. In embodiments, the implantable neuroregulator is configured to activate the first, second, and/or third therapy program if the blood pressure exceeds a high blood pressure threshold. In embodiments, the high blood pressure threshold is about 130 mm Hg systolic, 80 mmHg diastolic, or both. In embodiments, the therapeutic program selected will be tailored to the patient, and/or modified by the health care provider as a result of input from the sensor.

For example, a blood pressure of greater than about 120/80 mm Hg can result in an activation of the first, second, and/or third therapy program or a blood pressure of about 120/80 mm Hg or less can result in a temporary cessation of the first, second, and/or third therapy program. Likewise, a renin level of greater than 3 ng/ml/hr when a patient is standing may trigger an activation of the first, second, and/or third therapy program or a renin level of 3 ng/ml./hr or less can result in a temporary cessation of the first, second, and/or third therapy program. An aldosterone level of greater than 30 ng/dl when a patient is standing may trigger an activation of the first, second, and/or third therapy program or an aldosterone level of 30 ng/dl or less can result in a temporary cessation of the first, second, and/or third therapy program. An angiotensin II level of greater than about 0.3 micrograms per deciliter when a patient is standing may trigger an activation of the first, second, and/or third therapy program or angiotensin level of about 0.3 micrograms per deciliter or less can result in a temporary cessation of the first, second and/or third therapy program.

In embodiments, the neuroregulator and/or the external controller have programs and storage for the collection and transmission of sensed parameters such as heart rate, blood pressure, hormones, and oxygen saturation levels. Such data is communicated wirelessly to the external controller and/or a programmer so that therapy efficacy can be monitored and therapy program parameters changed to increase therapeutic efficacy or in response to an improvement in the patient's condition. For example, in a patient with hypertension and obesity, when a patient has stable systolic blood pressure of 120 mmHg or less and 80 mmHg diastolic blood pressure or less for at least 3 months, the therapy program may be selected to either be terminated or go into a maintenance mode.

v. Therapeutic Programs

In embodiments, the disclosure provides therapeutic programs that are tailored to the disease or condition of the patient. Therapeutic programs comprises parameters for electrical signal treatment. In embodiments, parameter values will vary depending on target nerve or on whether the electrical signal is an upregulating signal or downregulating signal. The healthcare provider may select a therapy program for each patient and may select individual parameters within each therapy program.

In some cases, as described herein, a first and/or second therapy program provides a down regulating signal to the vagus nerve at a location below the vagal innervation of the heart, to a sympathetic nerve or renal nerve. A third therapy program provides another signal applied elsewhere such as an up regulating signal applied at the right vagus nerve at SA node, to baroreceptors, or a glossopharyngeal nerve. In other embodiments, a first or second therapy program provides a down regulating signal to a vagus nerve and a third therapy program provides an upregulating signal is applied to baroreceptors.

In embodiments, for patients that have hypertension or heart failure without obesity or diabetes a therapeutic program involves parameters for providing an upregulating signal to a baroreceptor or glossopharyngeal nerve in combination with parameters providing an intermittent downregulating signal to the vagus at a subdiaphragmatic location and/or renal nerve. In other embodiments, the parameters for a therapeutic program providing a downregulating signal include a frequency of about 200 to 25 kHz, an on time of 30 seconds to 30 minutes, an off time of 30 seconds to 30 minutes, and an amplitude of 0.5 mA to 18 mA. In other embodiments, the parameters for a therapeutic program providing a downregulating signal to the renal nerve include a frequency of about 1000 Hz to 25 kHz, an on time of 30 seconds to 30 minutes, an off time of 30 seconds to 30 minutes, and an amplitude of about 3 mA to 18 mA.

An apparatus includes at least two electrodes. One electrode is adapted to contact the arterial baroreceptors and the other electrode is adapted to contact the vagus in a subdiaphragmatic location and/or renal nerve. In embodiments, the electrode adapted to contact the renal nerve is adapted to be placed on a blood vessel either externally or intravascularly. In embodiments, for placement on the renal nerve, the lead body includes a multitude of electrodes or contacts. When the lead body has a circular cross-sectional shape, the contacts can have a generally ring-type shape and can be spaced apart axially along the length of the lead body. In embodiments, the electrode adapted to contact baroreceptors is adapted to be placed on a blood vessel either external or intravascularly. In embodiments, an electrode is adapted for placement on the anterior or posterior vagal nerve below the diaphragm. In embodiments, the patient also is selected that has an increase in stiffening of the arteries as measured by aortic pulse wave velocity. The parameters for either therapeutic program are further selected to avoid adverse effects on heart rate or other cardiac function.

In embodiments, for patients that have hypertension or heart failure and obesity or diabetes, a therapeutic program comprises parameters selected to provide an intermittent downregulating signal to the vagus nerves subdiaphragmatically in combination with a downregulating signal on the renal nerve and/or spinal sympathetic nerves. In other embodiments, the parameters for such a therapeutic program include a frequency of 200 to 25 kHz, an on time of 30 seconds to 30 minutes, an off time of 30 seconds to 30 minutes, and an amplitude of 0.5 mA to 18 mA. In yet other embodiments, the therapeutic program further comprises parameters for a downregulating signal to a spinal splanchnic and/or renal nerve. A splanchnic nerve includes the first lumbar splanchnic nerve. In some cases, the parameters for downregulation of a splanchnic or renal nerve include a frequency of about 1000 Hz to 25 kHz, an on time of 30 seconds to 30 minutes, an off time of 30 seconds to 30 minutes, and an amplitude of about 3 mA to 18 mA. In other embodiments, a downregulating signal on any of the vagus, splanchnic, or renal nerve is combined with an upregulating signal on the baroreceptors.

In embodiments, for patients that have hypertension and/or chronic kidney disease with or without diabetes, a therapeutic program comprises parameters that provide for an intermittent downregulating signal to the renal and/or vagus nerve. In embodiments, for patients that have hypertension, heart failure and/or chronic kidney disease, a therapeutic program comprises parameters selected to provide an intermittent downregulating signal to the renal nerve independently of any other therapeutic program. In other embodiments, the parameters for such a therapeutic program include a frequency of 200 to 25 kHz, an on time of 30 seconds to 30 minutes, an off time of 30 seconds to 30 minutes, and an amplitude of 0.5 mA to 18 mA. In other embodiments, a downregulating signal on a renal nerve is coordinated to provide a down regulating signal to block synchronized burst of nerve activity, including such parameters as a frequency of about 1000 Hz to 25 kHz, an on time of 30 seconds to 30 minutes, an off time of 30 seconds to 30 minutes, and an amplitude of about 6 mA to 18 mA.

In yet other embodiments, in a normal weight hypertensive subject for which adequate blood pressure control has not been achieved with medication, at least one electrode is placed on renal nerve and therapy program selected to provide an intermittent down regulating signal to the renal nerve. In a further embodiment for treatment of such a subject, an electrode is placed in tissue or on a nerve or blood vessel that affects the baroreceptors and a therapy program selected to provide an upregulating signal to the tissue, nerve or blood vessel. In yet a further embodiment for treatment of a subject, an electrode is placed on spinal sympathetic nerve or a vagus nerve and a therapy program providing an intermittent downregulating signal is selected. Any combination of placement of electrodes and therapy programs can be selected. In addition, the therapy program or parameters of a therapy program may be modified as a result of sensed information and/or the health status of the patient during treatment.

In embodiments, one or more of the parameters are modified after treatment has begun in order to improve efficacy or patient compliance. Parameters that are modified by a health care provider include frequency, amplitude, on time, off time, pulse width, treatment period, ramp up time and ramp down time. Parameters may be modified in response to sensed patient status or the change in biomarkers. For example, if blood pressure exceeds a certain predetermined level, then a therapy program may be modified in response to that event. For example, in a patient with hypertension and obesity, when a patient has stable blood pressure of 120 mmHg or less and 80 mmHg or less for at least 3 months, the therapy program may be selected to either be terminated or go into a maintenance mode.

In embodiments, a maintenance mode is one in which the neuroregulator delivers low energy electrical signals associated with safety checks and impedance checks for a period of time of 9 hours or less. In the interest of conserving battery power, the device may remain on but deliver the safety and impedance checks for 30 minutes to 9 hours, 1 hour to 8 hours, 1 hour to 7 hours, 1 hour to 6 hours, 1 hour to 5 hours, 1 hour to 4 hours, 1 hour to 3 hours and 1 hour to 2 hours. In embodiments, the safety checks are delivered at 50 Hz or less at least every 0.2 μs and impedance checks are delivered once every two minutes at a frequency of 1000 Hz or more. While not meant to limit the scope of the invention, it is believed that a therapeutic effect is associated with this low energy electrical single treatment if applied for at least 9 hours per day and not at shorter time periods. If the patient condition has stabilized or resolved, a health care provider may program the device for maintenance mode, leaving open the option to initiate a therapy program once again at a later date.

In embodiments, biomarkers are evaluated in the patient and used to select the initial therapy program for the patient. For example, for patients that have hypertension, and an increased arterial stiffness as measured by, for example, the aortic pulse wave velocity, a therapy program is selected that includes an upregulating signal to baroreceptors. In other embodiments, for patients that have hypertension and a decrease in adiponectin, a therapy program is selected that includes parameters for downregulation of a vagus nerve. In yet other embodiments, for patients that have an increased level of cystatin C and hypertension, a therapy program is selected that provides a downregulating signal to a renal nerve. In yet other embodiments, for patients that have an increased level of C reactive protein and other inflammatory markers such as interleukin 6, a therapy program is selected that includes parameters for downregulation of a vagus nerve and a renal nerve.

Other biomarkers for arterial stiffness include imaging of the level of calcium deposits in blood vessels using coronary computed tomography angioplasty or other like procedures. Examination of an electrocardiogram is also useful to provide information about a wide variety of health parameters. Electrocardiogram signals can be analyzed using wavelet transformation technology as described in U.S. Pat. No. 7,082,327 and US 20100004515, which are hereby incorporated by reference.

In some embodiments, patients are assessed for psychiatric conditions using instruments such as the Beck Depression inventory and/or the Weight and Lifestyle Inventory (WALI). Patients exhibiting depression can be treated for depression before implantation and activation of the device.

Biomarkers can be monitored throughout treatment in order to assess whether modification in therapy programs and/or medication need to be made. In embodiments, a decrease in any one of cystatin C, C reactive protein, and/or interleukin 6 as compared to levels seen in subjects without obesity, diabetes, renal disease, and/or hypertension is indicative that the therapy is working and the therapy may be modified to a maintenance mode rather than a treatment mode. In embodiments, an increase in adiponectin as compared to a subject without hypertension is indicative that the therapy is working and the treatment program modified. Stabilization of blood pressure over at least a 3 month period may also warrant modification of the electrical signal treatment therapy to a maintenance mode.

vi. Selection of Pharmacological Agent

In another aspect of the disclosure, a pharmacological agent is selected for treatment of the patient for hypertension or heart failure in conjunction with the electrical signal therapy. In embodiments, the therapy apparatus, includes information about the patient and response to medications on blood pressure and heart rate parameters over a period of time including both the dose and timing of administration of the medication. Such information can be stored on the neuroregulator, the external charger and/or on a clinician programmer. This information can then be interrogated to assess the efficacy of the dose and timing of administration of the drug. In embodiments, this information is combined with information about blood pressure and heart rate obtained from a sensor, also sent to a health care provider so that adjustments can be made in the patient's medications and/or electrical signal treatment therapy.

Agents that affect impaired blood pressure control can be selected based on an ability to complement treatment of applying a signal to alter neural activity of a target nerve. As described herein, an agent is selected that may provide a complementary or synergistic effect with the application of signal to modulate neural activity on a target nerve such as the vagus nerve. A synergistic or complementary effect can be determined by determining whether the patient has an improvement in blood pressure and/or heart rate as described herein as compared to one or both treatments alone.

An agent may also or in addition be selected to be administered that may have undesirable side effects at the recommended dosage that prevents use of the agent, or that provides inadequate blood pressure control. In addition, patients that have cardiac conditions, liver disease, or renal disease may not be able to tolerate treatment with one or more of the agents at the recommended dosage due to adverse side effects.

Combining administration of a drug with undesirable side effects with modulating neural activity on a target nerve may allow for administration of the drugs at a lower dose thereby minimizing the side effects, may allow for administration of a single drug instead of multiple drugs, or may allow administration of higher doses of the drugs. In addition, a drug may be selected that has altered pharmacokinetics when absorption is slowed by a delay in gastric emptying due to neural downregulation as applied to a vagus nerve as described herein. In other embodiments, the recommended dosage may be lowered to an amount that has fewer adverse side effects. In embodiments, it is expected that the recommended dosage may be able to be lowered at least 25%. In other embodiments, the dosage can be lowered to any percentage of at least 25% or greater of the recommended dose. In some embodiments, the dosage is lowered at least 25, 30, 35, 40, 45, 50, 55, 60, 65, 70, 75, 80, 85, 90, 95, or 100% of the recommended dosage.

In embodiments, for patients that have impaired renal function and hypertension, an agent that affects the renin angiotensin pathway may be preferably selected. Such agents include angiotensin receptor blockers and angiotensin converting enzyme inhibitors. Likewise for patients that have increased levels of C reactive protein and hypertension, agents that affect atherosclerosis such as statins are preferably selected either alone or in combination with renin angiotensin inhibitors. In embodiments, for patients that are obese and have hypertension or heart failure, a combination of angiotensin renin inhibitors, beta blockers, and/or statins is preferred.

C. Methods

The disclosure provides methods of regulating heart rate and/or blood pressure. In some embodiments, a method comprises: applying an intermittent electrical signal to a target nerve at a site with said electrical signal selected to down-regulate and/or upregulate neural activity on the nerve and with neural activity at least partially restoring upon discontinuance of said signal. In some embodiments, the methods further comprise administering a composition to the subject comprising an effective amount of an agent that controls blood pressure or treats congestive heart failure. In some embodiments, the electrical signal is applied to the nerve by implanting a device or using an apparatus as described herein.

In some embodiments, a method of treating hypertension or pre-hypertension in a subject comprises applying an intermittent neural conduction signal to a target nerve of the subject having hypertension, with said neural conduction signal selected to down-regulate neural activity on the nerve and to at least partially restore neural activity on the nerve upon discontinuance of said signal. In other embodiments, the nerve conduction signal is applied continuously during the time of treatment. In embodiments, the treatment is applied to a renal nerve without any other upregulating or downregulating signal on the vagus nerve or other nerves. In embodiments, the treatment and/or signal characteristic are selected so that no other adverse clinical effects occur.

In some embodiments, a method of treating hypotension in a subject comprises applying an intermittent neural conduction signal to a target nerve of the subject having hypotension, with said neural conduction signal selected to up-regulate neural activity on the nerve and to at least partially restore neural activity on the nerve upon discontinuance of said signal. In other embodiments, the nerve conduction signal is applied continuously during the time of treatment.

In some embodiments, the disclosure provides a method of treating chronic kidney disease, comprising: applying an intermittent electrical treatment signal to a target nerve or blood vessel in proximity to the kidney of the subject wherein said electrical treatment signal is selected to at least partially down-regulate neural activity on the nerve during an on time and to at least partially restore neural activity on the nerve during an off time and wherein the on and off times are applied multiple times per day over multiple days. In other embodiments, the disclosure provides a method of treating chronic kidney disease, comprising: applying an intermittent electrical treatment signal to a target nerve or blood vessel in proximity to the kidney of the subject wherein said electrical treatment signal is selected to at least partially down-regulate neural activity on the nerve during an on time and to at least partially restore neural activity on the nerve during an off time and wherein the on and off times are applied multiple times per day over multiple days, wherein the down-regulation of the neural activity on the nerve is periodically adjusted to maintain the desired kidney function and avoid adaptation. In embodiments, the subject has chronic kidney disease without hypertension, obesity, or diabetes. In other embodiments, the subject has hypertension and chronic kidney disease.

In embodiments, for patients that have hypertension or heart failure without obesity or diabetes, a therapeutic program involves parameters for providing an upregulating signal to a baroreceptor in combination with parameters providing an intermittent downregulating signal to the vagus and/or renal nerve. In other embodiments, the parameters for a therapeutic program providing a downregulating signal include a frequency of 200 to 25 kHz, an on time of 30 seconds to 30 minutes, an off time of 30 seconds to 30 minutes, and an amplitude of 0.5 mA to 18 mA. In other embodiments, the parameters for a therapeutic program providing a downregulating signal to the renal nerve include a frequency of about 1000 Hz to 25 kHz, an on time of 30 seconds to 30 minutes, an off time of 30 seconds to 30 minutes, and an amplitude of about 6 mA to 18 mA. an apparatus includes at least two electrodes.

One electrode is adapted to contact the arterial baroreceptors and the other electrode is adapted to contact the vagus or renal nerve. In embodiments, the electrode adapted to contact the renal nerve is adapted to be placed on a blood vessel either externally or intravascularly. In embodiments, the electrode adapted to contact baroreceptors is adapted to be placed on a blood vessel either external or intravascularly. In embodiments, an electrode is adapted for placement on the anterior or posterior vagal nerve below the diaphragm. In embodiments, the patient also is selected that has an increase in stiffening of the arteries as measured by aortic pulse wave velocity. The parameters for either therapeutic program are further selected to avoid adverse effects on heart rate or other cardiac function.

In embodiments, for patients that have hypertension or heart failure and obesity, a therapeutic program comprises parameters selected to provide an intermittent downregulating signal to the vagus nerve independently of any other therapeutic program. In other embodiments, the parameters for such a therapeutic program include a frequency of 200 to 25 kHz, an on time of 30 seconds to 30 minutes, an off time of 30 seconds to 30 minutes, and an amplitude of 0.5 mA to 18 mA. In yet other embodiments, the therapeutic program further comprises parameters for a downregulating signal to a splanchnic and/or renal nerve. A splanchnic nerve includes the first lumbar splanchnic nerve. In some cases, the parameters for downregulation of a splanchnic or renal nerve include a frequency of about 1000 Hz to 25 kHz, an on time of 30 seconds to 30 minutes, an off time of 30 seconds to 30 minutes, and an amplitude of about 6 mA to 18 mA.

In embodiments, the electrical signal is applied to the renal nerve in a multiplex fashion where one series of pulses are delivered to the renal nerve with a first set of parameters followed by or interleaved with a second set of parameters. In embodiments, the first and second set of parameters only differ in a single parameter such as frequency or pulse amplitude. In a specific embodiment, a first set of pulses has a frequency of about 200 to 10,000 Hz followed by a second set of pulses at a frequency of 1 to 199 Hz. In other embodiments, more than one parameter differs between the first and second set of parameters. In embodiments, for patients that have hypertension or heart failure, diabetes, and obesity, a therapeutic program comprises parameters selected to provide an intermittent downregulating signal to the vagus nerve independently of any other therapeutic program. In other embodiments, the parameters for such a therapeutic program include a frequency of 200 to 25 kHz, an on time of 30 seconds to 30 minutes, an off time of 30 seconds to 30 minutes, and an amplitude of 0.5 mA to 18 mA. In yet other embodiments, the therapeutic program further comprises parameters for a downregulating signal to a splanchnic and/or renal nerve. A splanchnic nerve includes the first lumbar splanchnic nerve. In some cases, the parameters for downregulation of a splanchnic or renal nerve include a frequency of about 1000 Hz to 25 kHz, an on time of 30 seconds to 30 minutes, an off time of 30 seconds to 30 minutes, and an amplitude of about 6 mA to 18 mA. In other embodiments, a downregulating signal on any of the vagus, splanchnic, or renal nerve is combined with an upregulating signal on the baroreceptors.

In embodiments, for patients that have hypertension and chronic kidney disease with or without diabetes, a therapeutic program comprises parameters that provide for an intermittent downregulating signal to the renal and/or vagus nerve. In embodiments, for patients that have hypertension or heart failure and chronic kidney disease, a therapeutic program comprises parameters selected to provide an intermittent downregulating signal to the renal nerve independently of any other therapeutic program. In other embodiments, the parameters for such a therapeutic program include a frequency of 200 to 25 kHz, an on time of 30 seconds to 30 minutes, an off time of 30 seconds to 30 minutes, and an amplitude of 0.5 mA to 18 mA. In other embodiments, a downregulating signal on a renal nerve is coordinated to provide a down regulating signal to block synchronized burst of nerve activity, including such parameters as a frequency of about 1000 Hz to 25 kHz, an on time of 30 seconds to 30 minutes, an off time of 30 seconds to 30 minutes, and an amplitude of about 6 mA to 18 mA.

In yet other embodiments, in a normal weight hypertensive subject for which adequate blood pressure control has not been achieved with medication, at least one electrode is placed on renal nerve and therapy program selected to provide an intermittent down regulating signal to the renal nerve. In a further embodiment for treatment of such a subject, an electrode is placed in tissue or on a nerve or blood vessel that affects the baroreceptors and a therapy program selected to provide an upregulating signal to the tissue, nerve or blood vessel. In yet a further embodiment for treatment of a such a subject, an electrode is placed on sympathetic nerve or a vagus nerve and a therapy program providing an intermittent downregulating signal is selected. Any combination of placement of electrodes and therapy programs can be selected. In addition, the therapy program or parameters of a therapy program may be modified as a result of sensed information and/or the health status of the patient during treatment.

A therapeutic program can be designed for each patient depending on a change in biomarkers. For example, for patients that have hypertension, and an increased arterial stiffness as measured by, for example, the aortic pulse wave velocity, a therapy program is selected that includes an upregulating signal to baroreceptors. In other embodiments, for patients that have hypertension and a decrease in adiponectin, a therapy program is selected that includes parameters for downregulation of a vagus nerve. In yet other embodiments, for patients that have an increased level of cystatin C and hypertension, a therapy program is selected that provides a downregulating signal to a renal nerve. In yet other embodiments, for patients that have an increased level of C reactive protein and other inflammatory markers such as interleukin 6, a therapy program is selected that includes parameters for downregulation of a vagus nerve and a renal nerve.

Other biomarkers for arterial stiffness include imaging of the level of calcium deposits in blood vessels using coronary computed tomography angioplasty or other like procedures. Examination of an electrocardiogram is also useful to provide information about a wide variety of health parameters. Electrocardiogram signals can be analyzed using wavelet transformation technology as described in U.S. Pat. No. 7,082,327 and US 20100004515, which are hereby incorporated by reference.

Biomarkers can be monitored throughout treatment in order to assess whether modification in therapy programs and/or medication need to be made. In embodiments, a decrease in any one of cystatin C, C reactive protein, and/or interleukin 6 as compared to levels seen in subjects without obesity, diabetes, renal disease, and/or hypertension is indicative that the therapy is working and the therapy may be modified to a maintenance mode rather than a treatment mode. In embodiments, an increase in adiponectin as compared to a subject without hypertension is indicative that the therapy is working and the treatment program modified. Stabilization of blood pressure over at least a 3 month period may also warrant modification of the electrical signal treatment therapy to a maintenance mode.

In embodiments, one or more of the parameters are modified after treatment has begun in order to improve efficacy or patient compliance. Parameters that are modified by a health care provider include frequency, amplitude, on time, off time, pulse width, treatment period, ramp up time and ramp down time. Parameters may be modified in response to sensed patient status or the change in biomarkers. If biomarkers indicate patient improvement, the therapy program may be terminated or changed to maintenance mode. For example, if blood pressure exceeds a certain predetermined level, then a therapy program may be modified in response to that event. In other embodiments, if the patient's condition improves and a stable blood pressure at or below 120 mmHg and 80 mmHg for at least 3 months, the therapeutic program can be terminated or switched to maintenance mode.

In embodiments, a maintenance mode is one in which the neuroregulator delivers low energy electrical signals associated with safety checks and impedance checks for a period of time of 9 hours or less. In the interest of conserving battery power, the device may remain on but deliver the safety and impedance checks for 30 minutes to 9 hours, 1 hour to 8 hours, 1 hour to 7 hours, 1 hour to 6 hours, 1 hour to 5 hours, 1 hour to 4 hours, 1 hour to 3 hours and 1 hour to 2 hours. In embodiments, the safety checks are delivered at 50 Hz or less at least every 0.2 μs and impedance checks are delivered once every two minutes at a frequency of 1000 Hz or more. While not meant to limit the scope of the invention, it is believed that a therapeutic effect is associated with this low energy electrical single treatment if applied for at least 9 hours per day and not at shorter time periods. If the patient condition has stabilized or resolved, a health care provider may program the device for maintenance mode, leaving open the option to initiate a therapy program once again at a later date.

In other embodiments, methods include a treatment for hypertension, congestive heart failure, pre-hypertension, or other conditions having hypertension as a component, comprising selecting a drug for treating hypertension, congestive heart failure, or other condition for a patient where effective dosages for treating such conditions for such a patient are associated with disagreeable side effects or impaired blood pressure control; and treating the patient with a concurrent treatment comprising: a) applying an intermittent neural block to a target nerve of the patient at multiple times per day and over multiple days with the block selected to down-regulate afferent and/or efferent neural activity on the nerve and with neural activity at least partially restoring upon discontinuance of said block; and b) administering said drug to the patient.

Another method includes a method of manufacturing an apparatus comprising: configuring the implantable neuroregulator to deliver a first therapy program to the first target nerve or blood vessel, wherein the first therapy program delivers an electrical signal to the first target nerve or blood vessel intermittently with an on time and an off time multiple times in a day, wherein the first therapy program delivers an electrical signal treatment that has a frequency selected to down regulate neural activity on the first nerve or blood vessel during an on time and has an off time selected to provide for at least partial recovery of nerve function; configuring the implantable neuroregulator to deliver a third therapy program to the second target nerve or tissue, wherein the third therapy program delivers an electrical signal to second target nerve or blood vessel intermittently with an on time and an off time multiple times in a day, wherein the third therapy program delivers an electrical signal treatment that has a frequency to up regulate neural activity; and c) configuring the implantable neuroregulator to operate in selectable multiple modes comprising a first mode comprising providing the first therapy program to the first and additional electrode, a second mode comprising providing the first therapy program to the first electrode and the third therapy program to the additional electrode, and a maintenance mode. In embodiments, the first and third therapy programs are configured to be delivered during the same on time or at different on times.

In another embodiment, the method includes providing a first electrode adapted to be placed on a first target nerve or blood vessel selected from the group consisting of renal artery, vagus nerve, renal nerve, vagus nerve, celiac plexus, a splanchnic nerve, cardiac sympathetic nerves, and spinal nerves originating between T10 to L5. In yet further embodiments, a method provides an additional electrode adapted to be placed on a first target nerve or blood vessel selected from the group consisting of renal artery, renal nerve, vagus nerve, celiac plexus, a splanchnic nerve, cardiac sympathetic nerves, spinal nerves originating between T10 to L5, glossopharyngeal nerve, and tissue containing baroreceptors.

In embodiments, a method includes providing a sensor, wherein the sensor detects a parameter selected from the group consisting of blood pressure, heart rate, mean arterial pressure, hormones, and combinations thereof In embodiments, the method includes configuring the implantable neuroregulator to activate the first, second, and/or third therapy program if the blood pressure exceeds a high blood pressure threshold.

i. Signal Application

In one aspect of the disclosure a reversible intermittent modulating signal is applied to a target nerve in order to downregulate and/or upregulate neural activity on the nerve. In other embodiments, a signal is applied to a target nerve to upregulate or downregulate neural activity continuously during the treatment time. In embodiments, the target nerve is the vagus nerve.

In embodiments of the methods described herein a neural conduction block is applied to a target nerve at a site with said neural conduction block selected to down-regulate neural activity on the nerve and with neural activity at least partially restoring upon discontinuance of said signal.

In some embodiments, said modulating signal comprises applying an electrical signal. The signal is selected to down regulate or up regulate neural activity and allow for at least partial restoration of the neural activity upon discontinuance of the signal. A neuroregulator, as described above, can be employed to regulate the application of the signal in order to alter the characteristic of the signal to provide a reversible intermittent signal. The characteristics of the signal include location of the signal, frequency of the signal, amplitude of the signal, voltage of the signal, pulse width of the signal, ramp-up and ramp-down characteristics and the administration cycle of the signal. In some embodiments, the signal characteristics are selected to provide for improved heart rate and/or blood pressure.

In some embodiments, electrodes applied to a target nerve are energized with an intermittent blocking or down regulating signal. The signal is applied for a limited time (e.g., 5 minutes). The speed of neural activity recovery varies from subject to subject. However, 20 minutes is a reasonable example of the time needed to recover to baseline. After recovery, application of a blocking signal again down-regulates neural activity which can then at least partially recover after cessation of the signal. Renewed application of the signal can be applied before full recovery. For example, after a limited time period (e.g., 10 minutes) blocking can be renewed resulting in average neural activity not exceeding a level significantly reduced when compared to baseline. In some embodiments, the electrical signal is applied intermittently in a cycle including an on time of application of the signal followed by an off time during which the signal is not applied to the nerve, wherein the on and off times are applied multiple times per day over multiple days

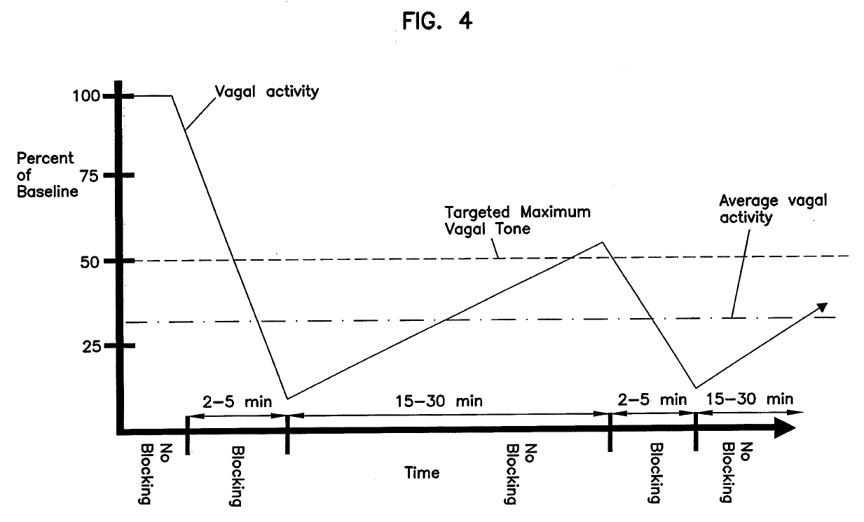

Recognition of recovery of neural activity, such as vagal activity, permits a treatment therapy and apparatus with enhanced control and enhanced treatment options. FIG. 4 illustrates vagal activity over time in response to application of a blocking signal as described above and further illustrates recovery of vagal activity following cessation of the blocking signal. It will be appreciated that the graph of FIG. 4 is illustrative only. It is expected there will be significant patient-to-patient variability. For example, some patients' responses to a blocking signal may not be as dramatic as illustrated. Others may experience recovery slopes steeper or shallower than illustrated. Also, vagal activity in some subjects may remain flat at a reduced level before increasing toward baseline activity. However, based on the afore-mentioned animal experiments, FIG. 4 is believed to be a fair presentation of a physiologic response to blocking.

In FIG. 4, vagal activity is illustrated as a percent of baseline (i.e., vagal activity without the treatment of the present invention). Vagal activity can be measured in any number of ways. For example, quantities of pancreatic exocrine secretion produced per unit time are an indirect measurement of such activity. Also, activity can be measured directly by monitoring electrodes on or near the vagus. Such activity can also be ascertained qualitatively (e.g., by a patient's sensation of bloated feelings or normalcy of gastrointestinal motility).

In FIG. 4, the vertical axis is a hypothetical patient's vagal activity as a percent of the patient's baseline activity (which varies from patient to patient). The horizontal axis represents the passage of time and presents illustrative intervals when the patient is either receiving a blocking signal as described or the blocking signal is turned off (labeled “No Blocking”). As shown in FIG. 4, during a short period of receiving the blocking signal, the vagal activity drops dramatically (in the example shown, to about 10% of baseline activity). After cessation of the blocking signal, the vagal activity begins to rise toward baseline (the slope of the rise will vary from patient to patient). The vagal activity can be permitted to return to baseline or, as illustrated in FIG. 4, the blocking signal can be re-instituted when the vagal activity is still reduced. In FIG. 4, the blocking signal begins when the vagal activity increases to about 50% of baseline. As a consequence, the average vagal activity is reduced to about 30% of the baseline activity. It will be appreciated that by varying the blocking time duration and the “no blocking” time duration, the average vagal activity can be greatly varied.

The signal may be intermittent or continuous. The preferred nerve conduction block is an electronic block created by a signal at the target nerve by an electrode controlled by the implantable neuroregulator (such as neuroregulator 104 or an external controller). Electronic blocks can include low frequency baseline modulation. The nerve conduction block can be any reversible block. For example, ultrasound, alteration in temperature, or drug blocks can be used. An electronic block may be a Peltier solid-state device which cools in response to a current and may be electrically controlled to regulate cooling. Piezo-electric devices can be used to apply a mechanical energy to the nerve(s) to modulate activity. Drug blocks may include a pump-controlled subcutaneous drug delivery. Different types of neural activity blocks can be applied to different target nerves or blood vessels.

With such an electrode conduction block, the block parameters (signal type and timing) can be altered by the neuroregulator and can be coordinated with the upregulating signals. For example, the nerve conduction block parameters for muscles are disclosed in Solomonow, et al., “Control of Muscle Contractile Force through Indirect High-Frequency Stimulation”, Am. J. of Physical Medicine, Vol. 62, No. 2, pp. 71-82 (1983). In some embodiments, the nerve conduction block is applied with electrical signal selected to block the entire cross-section of the nerve (e.g., both afferent, efferent, myelinated and nomnyelinated fibers) at the site of applying the blocking signal (as opposed to selected sub-groups of nerve fibers or just efferent and not afferent or vice versa) and, more preferably, has a frequency selected to exceed the 200 Hz threshold frequency. Further, more preferred parameters are a frequency of 5000 Hz (with other parameters, as non-limiting examples, being amplitude of 6 mA, pulse width of 0.09 msec, and duty cycle of 5 minutes on and 5 minutes off). As will be more fully described, the present invention gives a physician great latitude in selected pacing and blocking parameters for individual patients.

In embodiments, the signal parameters provide for a decrease in heart rate and/or blood pressure, preferably without affecting other cardiac functions. The frequency is selected to provide at least a partial decrease in activity of the nerve. In some embodiments, the neuroregulator is configured to deliver a signal of about 200 Hz to 25 kHz, 200 Hz to about 15 kHz, 200 Hz to about 10 kHz, 200 to 5000 Hz, 250 to 5000 Hz, 300 to 5000 Hz, 400 to 5000 Hz, 500 to 5000 Hz, 200 to 2500 Hz, 300 to 2500 Hz, 400 to 2500 Hz, 500 to 2500 Hz, and any frequencies in between 200 Hz to 25 kHz or combinations thereof.

In embodiments, nerve activity can be blocked using low frequency baseline modulation. For example, in the initial negative portion of a biphasic pulse, the amplitude is increased (or could be decreased) by (for example) 100 μA, producing a direct current offset which could be effective in achieving a neural block. In the subsequent positive portion of the biphasic pulse, a compensatory amplitude is increased by the same 100 μA, also producing a direct current offset which could be effective in achieving a neural block, and ensuring that the net current/charge transmitted to the tissue during one biphasic pulse cycle, is zero. In other embodiments, increased (or decreased) pulse widths in the negative and positive regions of the biphasic pulse achieves the same effect of direct current/charge offset, while maintaining the net charge per biphasic pulse cycle, at zero.

In some cases, a downregulating signal is applied to a vagus nerve, a splanchnic nerve, a spinal sympathetic nerve, or a renal nerve either independently or in combination.

In embodiments, when sympathetic nerve activity is modulated, the timing and frequency of the electrical signal treatment is modified in order to at least partially block rapid synchronized bursts of nerve activity. In embodiments, the electrical signal is applied to the renal nerve in a multiplex fashion where one series of pulses are delivered to the renal nerve with a first set of parameters followed by or interleaved with a second set of parameters. In embodiments, the first and second set of parameters only differ in a single parameter such as frequency or pulse amplitude. In a specific embodiment, a first set of pulses has a frequency of about 200 to 10,000 Hz followed by a second set of pulses at a frequency of 1 to 199 Hz. In other embodiments, more than one parameter differs between the first and second set of parameters.

The signal is intermittent with an “on time and an off” time. In embodiments, each ON time includes a ramp-up where the 5,000 Hz signal is ramped up from zero amperes to a target of 6-8 mA. Each ON time further includes a ramp-down from full current to zero current at the end of the ON time. For about 50% of the patients, the ramp durations were 20 seconds and for the remainder the ramp durations were 5 seconds. In some embodiments, the on time is elected to have a duration of no less than 30 seconds or no more than 180 seconds or both. The duration of the on time is selected to provide for at least partial blocking or downregulation of the neural activity. The off time is selected to provide for at least partial recovery of neural activity.

The use of ramp-ups and ramp-downs are conservative measures to avoid possibility of patient sensation to abrupt application or termination of a full-current 5,000 Hz signal.

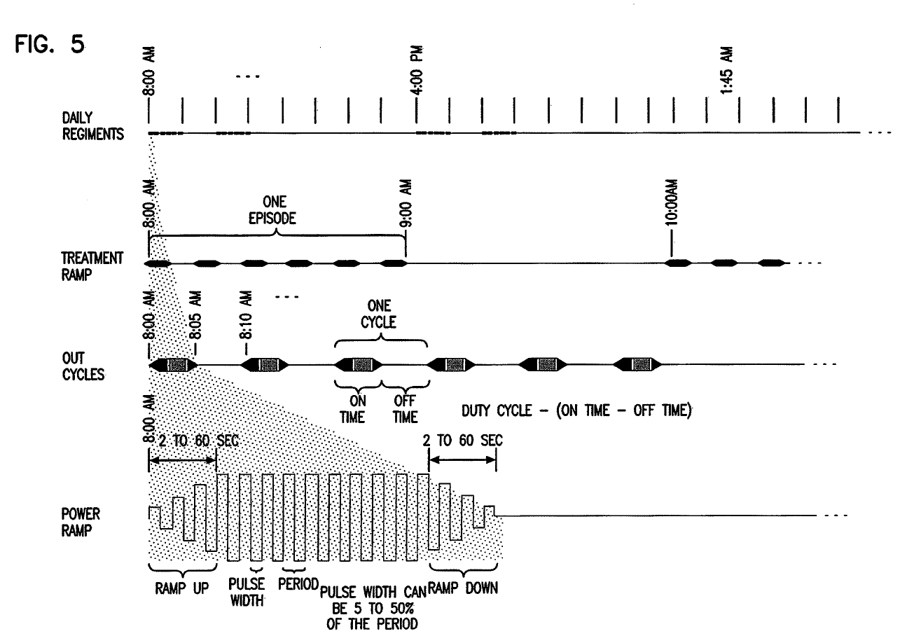

In some embodiments, a mini duty cycle can be applied. In an embodiment, a mini duty cycle comprises 180 millisecond periods of mini-ON times of 5,000 Hz at a current which progressively increases from mini-ON time to mini-ON time until full current is achieved (or progressively decreases in the case of a ramp-down). Between each of such mini-ON times, there is a mini-OFF time which can vary but which is commonly about 20 milliseconds in duration during which no signal is applied. Therefore, in each 20-second ramp-up or ramp-down, there are approximately one hundred mini-duty cycles, having a duration of 200 milliseconds each and each comprising approximately 180 milliseconds of ON time and approximately 20 milliseconds of OFF time. A representative duty cycle is shown in FIG. 5.

The on times are selected to provide at least a partial decrease in nerve activity. In embodiments, the neuroregulator is configured to deliver on times of from 30 seconds to 30 minutes, 30 seconds to 20 minutes, 30 seconds to 10 minutes, 30 seconds to 5 minutes, 30 sec to 3 minutes, 30 seconds to 2 minutes, or 30 seconds to 1 minute or combinations thereof. The off times are selected to allow at least partial recovery of the nerve activity. In embodiments, the neuroregulator is configured to deliver off times of from 30 seconds to 30 minutes, 30 seconds to 20 minutes, 30 seconds to 10 minutes, 30 seconds to 5 minutes, 30 sec to 3 minutes, 30 seconds to 2 minutes, or 30 seconds to 1 minute or combinations thereof.

In other embodiments other on times and off time may be utilized as appropriate for the patient's condition and responsiveness to treatment. For example, the on times may be 30 minutes or longer followed by an off time of at least 30 minutes, or an on time of at least 30 minutes followed by an off time of 24 hours or longer. A specific embodiment includes one or more therapy on periods of at least 30 minutes with intervening therapy off periods for up to 7 days or longer.

In embodiments, the current and/or voltage are adjusted based on safety and efficacy of treatment for the patient. In some embodiments, the signal amplitude can range from 0.5 mA to about 18 mA including amplitudes in between that differ by 0.25 mA, or other larger or smaller increments, adjusted up or down based on patient response. Voltages can range from 0.25 volts up to 20 volts or voltages in between that differ by 0.25 volts, or other larger or smaller increments, adjusted up or down based on patient response.

The treatment time can be an entire 24 hour period, 18 to 24 hours, 16 to 24 hours, 12 to 24 hours, and 9 to 24 hours, 6 to 24 hours, 4 to 24 hours or other intervals that match the treatment needs and/or activities of daily living of the patient or combinations thereof. Treatment time may be varied depending on whether the patient experiences a drop in blood pressure while sleeping. (Pickering et al, N. Eng. J. Med. 354:22 (2002)). Some patients who have hypertension have a blood pressure of greater than or equal to 135/85 mm Hg while they are awake and greater than or equal to 120/75 mm Hg when they are asleep. For those patients, the treatment would not be administered during some of the sleeping hours of the patient. However, in most cases, treatment would resume as early as 4 am in order to minimize the early morning spike in blood pressure that can lead to heart attack or stroke. (Pickering et al, cited supra) In other cases, for those patients who do not experience a drop in blood pressure while they are sleeping, treatment may be administered for a full 24 hour period.

In embodiments, a down regulating signal is applied to the vagus nerve at a location below the vagal innervation of the heart. In other embodiments, a down regulating signal is applied to the vagus nerve at a location below the vagal innervation of the heart and a down regulating signal is applied to a sympathetic nerve innervating the heart.

In embodiments of the methods described herein a signal is applied to a target nerve at a site with said signal selected to up-regulate neural activity on the nerve and with neural activity at least partially restoring upon discontinuance of said signal. In some embodiments, an upregulating signal may be applied in combination with a down regulating signal in order to improve heart rate and/or blood pressure.

The signal is selected to upregulate neural activity and allow for restoration of the neural activity upon discontinuance of the signal. To decrease heart rate and blood pressure, an upregulating signal may be applied at the right vagus nerve near the SA node of the heart or an upregulating signal may be applied to the baroreceptors. A neuroregulator, as described above, is employed to regulate the application of the signal in order to alter the characteristic of the signal to provide a reversible intermittent signal. The characteristics of the signal include frequency of the signal, location of the signal, and the administration cycle of the signal.

In some embodiments, electrodes applied to a target nerve are energized with an up regulating signal. The signal is applied for a limited time (e.g., 5 minutes). The speed of neural activity recovery varies from subject to subject. However, 20 minutes is a reasonable example of the time needed to recover to baseline. After recovery, application of an up signal again up-regulates neural activity which can then recover after cessation of the signal. Renewed application of the signal can be applied before full recovery. For example, after a limited time period (e.g., 10 minutes) upregulating signal can be renewed. Frequencies for upregulation include frequencies of about 0 to 200 Hz, 1 to 150 Hz, 1 to 100 Hz, 1 to 75 Hz, 1 to 50 Hz, 1 to 25 Hz, or combinations thereof.

In some embodiments, an upregulating signal may be applied in combination with a down regulating signal in order to improve heart rate and/or blood pressure. The upregulating and down regulating signals may be applied to different nerves at the same time, applied to the same nerve at different times, or applied to different nerves at different times. For example, a downregulating signal may be applied during the day when blood pressure tends to be higher, followed by a stimulatory signal while sleeping.

Normally a patient would only use the device while awake. The hours of therapy delivery can be programmed into the device by the clinician (e.g., automatically turns on at 5:00 AM and automatically turns off anywhere from 10 pm to 1:00 am). In some cases, the hours of therapy would be modified to correspond to times when blood pressure fluctuates such as during the day. For example, the hours of therapy may be adjusted to start at early in the morning when heart attack and stroke are more likely to occur. In embodiments, the device is configured to deliver therapy no less than 12 hours while the patient is awake.

The treatment time can be an entire 24 hour period, 18 to 24 hours, 16 to 24 hours, 12 to 24 hours, 9 to 24 hours, 6 to 24 hours, 4 to 24 hours, or any interval that provides for patient responsiveness, or combinations thereof. Treatment time may be varied depending on whether the patient experiences a drop in blood pressure while sleeping. Some patients who have hypertension have a blood pressure of greater than or equal to 135/85 mm Hg while they are awake and greater than or equal to 120/75 mm Hg when they are asleep. For those patients, the treatment would not be administered during some of the sleeping hours of the patient. However, in most cases, treatment would resume as early as 4 am in order to avoid the early morning spike in blood pressure which can lead to heart attack or stroke. In other cases, for those patients who do not experience a drop in blood pressure while they are sleeping, treatment may be administered for a full 24 hour period.

In the RF-powered version of the neuroregulator, use of the device is subject to patient control. For example, a patient may elect to not wear the external antenna. The device keeps track of usage by noting times when the receiving antenna is coupled to the external antenna through radio-frequency (RF) coupling through the patient's skin.

In some cases, loss of signal contact between the external controller 101 and implanted neuroregulator 104 occurs in large part to misalignment between coils 102, 105. It is believed coil misalignment results from, at least in part, changes in body surface geometry throughout the day (e.g., changes due to sitting, standing or lying down). These changes can alter the distance between coils 102, 105, the lateral alignment of the coils 102, 105 and the parallel alignment of the coils 102, 105. Misalignment can be detected by the device and alignment of the coils adjusted by the patient of physician to ensure that the signals are restored. The device may include a notification to the patient or physician if there has been a misalignment.

In some embodiments, the external component 101 can interrogate the neuroregulator component 104 for a variety of information. In some embodiments, therapy times of 30 seconds to 180 seconds per duty cycle are preferred to therapy times of less than 30 seconds per duty cycle or greater than 180 seconds per duty cycle.

During a 10 minute duty cycle (i.e., intended 5 minutes of therapy followed by a 5 minute OFF time), a patient can have multiple treatment initiations. For example, if, within any given 5-minute intended ON time, a patient experienced a 35-second ON time and 1.5 minute actual ON time (with the remainder of the 5-minute intended ON time being a period of no therapy due to signal interruption), the patient could have two actual treatment initiations even though only one was intended. The number of treatment initiations varies inversely with length of ON times experienced by a patient.

The flexibility to vary average neural activity, such as vagal activity, gives an attending physician great latitude in treating a patient. For example, in treating hypertension, the blocking signal can be applied with a short “no blocking” time. If the patient experiences discomfort, the duration of the “no blocking” period can be increased to improve patient comfort. The blocking and no blocking duration can be adjusted to achieve patient comfort. Other parameters can be adjusted including current amplitude and frequency.

While patient comfort may be adequate as feedback for determining the proper parameters for duration of blocking and no blocking, more objective tests can be developed. For example, the duration of blocking and no blocking can be adjusted to achieve desired levels of blood pressure control. Such testing can be measured and applied on a per patient basis or performed on a statistical sampling of patients and applied to the general population of patients.

In some embodiments, a sensor may be employed. A sensing electrode SE can be added to monitor neural activity as a way to determine how to modulate the neural activity and the duty cycle. While sensing electrode can be an additional electrode to blocking electrode, it will be appreciated a single electrode could perform both functions. The sensing and blocking electrodes can be connected to a controller as shown in FIG. 1. Such a controller is the same as controller 102 previously described with the additive function of receiving a signal from sensing electrode. In addition, the sensor may be a sensor external to the body and able to measure blood pressure, heart rate, and blood oxygen saturation, and communicate to the therapeutic apparatus wirelessly.

In some embodiments, the sensor can be a sensing electrode, a sensor, or sensor that senses other biological molecules or hormones of interest. A sensor may also be employed to measure heart rate, blood pressure, or cardiac function or any combination thereof. When the sensing electrode SE yields a signal representing a preselected blood pressure (e.g. greater than or equal to 130 mm Hg and/or greater than or equal to 80 mm Hg) or a targeted maximum vagal activity or tone (e.g., 50% of baseline as shown in FIG. 4) the controller with the additive function of receiving a signal from sensing electrode energizes the blocking electrode BE with a blocking signal. As described with reference to controller 102, a controller with the additive function of receiving a signal from a sensing electrode can be remotely programmed as to parameters of blocking duration and no blocking duration as well as targets for initiating a blocking signal.

In embodiments, the sensor is an external sensor that an measure heart rate, blood pressure, oxygen saturation, and blood glucose (for example in tears)and communicate this information to the neuroregulator, external controller, and/or clinician programmer. Such information is useful to modify electrical signal treatment therapy and/or drug therapy.

ii. Agents that Alter Blood Pressure of the Subject

The disclosure provides methods for treating a condition associated with impaired blood pressure and/or heart rate that include administering to a subject a composition comprising an agent that affects blood pressure and/or heart rate in a subject. In some embodiments, the patients may be refractory to one or more pharmaceuticals for treatment of elevated blood pressure. In that case, modulation of vagal nerve activity may be employed without administration of other agents. In other cases, for patients refractory to one or more drugs a combination of modulation of vagal nerve activity with administration of one or more agents may be beneficial. In other embodiments, a drug used to treat a cardiac condition may be associated with hypotensive effects and therefore the drug may be administered with an electrical treatment signal that increases blood pressure.

Agents that affect impaired blood pressure control can be selected based on an ability to complement treatment of applying a signal to alter neural activity of a target nerve. As described herein, an agent is selected that may provide a complementary or synergistic effect with the application of signal to modulate neural activity on a target nerve such as the vagus nerve. A synergistic or complementary effect can be determined by determining whether the patient has an improvement in blood pressure and/or heart rate as described herein as compared to one or both treatments alone.

In some embodiments, agents that act at a different site or through a different pathway may be selected for use in the methods described herein. Agents that complement treatment are those that include a different mechanism of action for affecting the heart rate and/or blood pressure control of the subject.

An agent may also or in addition be selected to be administered that may have undesirable side effects at the recommended dosage that prevents use of the agent, or that provides inadequate blood pressure control. In addition, patients that have cardiac conditions, liver disease, or renal disease may not be able to tolerate treatment with one or more of the agents at the recommended dosage due to adverse side effects.

Combining administration of a drug with undesirable side effects with modulating neural activity on a target nerve may allow for administration of the drugs at a lower dose thereby minimizing the side effects, may allow for administration of a single drug instead of multiple drugs, or may allow administration of higher doses of the drugs. In addition, a drug may be selected that has altered pharmacokinetics when absorption is slowed by a delay in gastric emptying due to neural downregulation as described herein. In other embodiments, the recommended dosage may be lowered to an amount that has fewer adverse side effects. In embodiments, it is expected that the recommended dosage may be able to be lowered at least 25%. In other embodiments, the dosage can be lowered to any percentage of at least 25% or greater of the recommended dose. In some embodiments, the dosage is lowered at least 25, 30, 35, 40, 45, 50, 55, 60, 65, 70, 75, 80, 85, 90, 95, or 100% of the recommended dosage.

In an embodiment, a method provides a treatment for a condition associated with impaired blood pressure and/or heart rate. Conditions associated with impaired blood pressure and/or heart rate includes, hypertension, prehypertension, congestive heart failure, ischemic heart disease, coronary artery disease, chronic renal disease, and cerebral vascular disease. A method comprises selecting a drug useful for treating hypertension or congestive heart failure and having a recommended dosage for efficacy where a patient is likely to experience disagreeable side effects at said recommended dosage; and treating the patient with a concurrent treatment comprising: applying an intermittent neural block to a target nerve of the patient at multiple times per day and over multiple days with the block selected to down-regulate afferent and/or efferent neural activity on the nerve and with neural activity restoring upon discontinuance of said block; and administering said drug to the patient at a dosage less than said recommended dosage. In some embodiments, the effective dosages for such a patient are associated with disagreeable side effects contributing to said patient not complying with a drug treatment. In some embodiments, patients are those that have cardiac conditions, liver, or renal disorder and may not be able to tolerate treatment with one or more of the agents.

A method comprises selecting a drug useful for treating a cardiac condition and having a recommended dosage for efficacy where a patient is likely to experience disagreeable side effects at said recommended dosage such as hypotension; and treating the patient with a concurrent treatment comprising: applying an intermittent neural conduction signal to a target nerve of the patient at multiple times per day and over multiple days with the signal selected to up-regulate neural activity and with neural activity restoring upon discontinuance of said signal; and administering said drug to the patient at a dosage less than said recommended dosage. In embodiments, the target nerve is the vagus nerve at a location below vagal innervation of the heart.

A number of oral and parenteral medications are available for the treatment of hypertension. Some of these medications are also commonly employed for the treatment of congestive heart failure.

Beta-blockers (beta-adrenergic blockers) work by reducing sympathetic nerve input to the heart. Thus, the heart beats less often per minute and with less force. Subsequently, the heart reduces its work, and blood pressure drops. Beta-blockers include propranolol, metoprolol, atenolol, and many others. Alpha-blockers (alpha-adrenergic blockers) target the nervous apparatus to relax blood vessels, allowing blood to pass more easily. Examples of alpha blockers are doxazosin, prazosin, and terazosin. Alpha-beta-blockers (alpha- and beta-adrenergic blockers) basically have the same effect as a combined alpha-blocker and beta-blocker. They target the nervous apparatus to relax the blood vessels, as well as work to slow the heartbeat. As a result, less blood is pumped through wider vessels, decreasing the overall blood pressure. Alpha-beta-blockers include labetalol and carvedilol.

Diuretics cause the body to excrete water and salt. This leads to a reduction in plasma volume, which subsequently lowers systemic blood pressure. Diuretics include furosemide, hydrochlorothiazide, and spironolactone.

Angiotensin Converting Enzyme (ACE) inhibitors work by preventing the body's production of angiotensin II, a hormone that normally causes blood vessels to narrow. Consequently, the vessels remain wider, which lowers blood pressure. Angiotensin II also normally stimulates the release of another hormone, called aldosterone, which is responsible for the body's retention of sodium. Hence, in addition to creating wider vessels, ACE inhibitors mimic the effect of diuretics to a certain extent. As a result, blood vessels are subject to less pressure, and the heart performs less work. Examples of ACE inhibitors include enalapril, captopril, and lisinopril. Angiotensin II antagonists are primarily used for patients who develop a cough as a side effect of taking ACE inhibitors. This medication antagonizes angiotensin II, thus inhibiting its effects. Examples include losartan and valsartan.

Calcium channel blockers keep calcium from entering the muscle cells of the heart and blood vessels. The heart and vessels relax, allowing blood pressure to go down. Some calcium channel blockers are nifedipine, verapamil, and diltiazem.

Vasodilators work by relaxing the muscle in the blood vessel wall. Hydralazine and minoxidil are both generic forms of vasodilators.

All drugs used for hypertension or congestive heart failure have side effects. Common side effects include fatigue, coughing, skin rash, sexual dysfunction, depression, cardiac dysfunction, or electrolyte abnormalities. In addition, some of the drugs may not be compatible with other drugs that are administered to people with cardiac problems. Ongoing patient compliance may be difficult. Some clinicians have been concerned about the long-term effects of anti-hypertensive drugs on mental processes.

Dosages for administration to a subject can readily be determined by one of skill in the art. Guidance on the dosages can be found, for example, by reference to other drugs in a similar class of drugs. For example, dosages have been established for any of the approved drugs or drugs in clinical trials and the range of dose will depend on the type of drug. Dosages associated with adverse side effects are known or can also be readily determined based on model studies. A determination of the effective doses to achieve improved blood pressure control while minimizing side effects can be determined by animal or human studies.

Agents will be formulated, dosed, and administered in a fashion consistent with good medical practice. Factors for consideration in this context include the particular disorder being treated, the clinical condition of the individual patient, the cause of the disorder, the site of delivery of the agent, the method of administration, the scheduling of administration, and other factors known to medical practitioners. The agent need not be, but is optionally formulated with one or more agents currently used to prevent or treat the disorder in question. The effective amount of such other agents depends on the amount of agent that improves glycemic control of the subject present in the formulation, the type of disorder or treatment, and other factors discussed above. These are generally used in the same dosages and with administration routes as used hereinbefore or about from 1 to 99% of the heretofore employed dosages.

Therapeutic formulations comprising the agent are prepared for storage by mixing the agent having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated. In such embodiments, the compounds have complementary activities that do not adversely affect each other. Such molecules are suitably present in combination in amounts that are effective for the purpose intended.

The therapeutic agent is/are administered by any suitable means, including parenteral, subcutaneous, orally, intradermal, intraperitoneal, and by aerosol. Parenteral infusions include intramuscular, intravenous, intraarterial, intraperitoneal, or subcutaneous administration. Pumps may be utilized as well as drug eluting devices and capsules.

### EXAMPLE 1

### Material and Methods/Experimental Design

An open-label, prospective, baseline-controlled, four-center clinical study was conducted to evaluate feasibility and safety and efficacy of a device as described herein that causes intermittent electrical blocking of the anterior and posterior vagal trunks. The participating centers included Flinders Medical Centre, Adelaide, Australia; Circle of Care, Sydney, Australia; University Hospital, Basel, Switzerland; and St. Olays University Hospital, Trondheim, Norway.

Patients

Male or female obese subjects (BMI 31.5-55 kg/m²) 25-60 years of age inclusive, were recruited at the four centers. The study assessed device safety and efficacy for 6 months.

Ability to complete all study visits and procedures was an eligibility requirement. Relevant exclusion criteria included: current type 1 diabetes mellitus (DM) or type 2 DM poorly controlled with oral hypoglycemic agents or with associated autonomic neuropathy, including gastroparesis; treatment with weight-loss drug therapy or smoking cessation within the prior three months or reductions of more than 10% of body weight in the previous 12 months; prior gastric resection or other major abdominal surgery, excluding cholecystectomy and hysterectomy; clinically significant hiatal hernias or intra-operatively determined hiatal hernia requiring surgical repair or extensive dissection at esophagogastric junction at time of surgery; and presence of a permanently implanted electrical powered medical device or implanted gastrointestinal device or prosthesis.

Concurrent treatment for thyroid disorders, epilepsy or depression with tricyclic agents was acceptable for participation if the treatment regimen was stable for the prior six months.

Implantation of Device

The device included two electrodes (one for each vagal trunk), a neuroregulator (neuroregulator) placed subcutaneously and an external controller to program the device.

Under general anesthesia, two leads (electrodes) of the vagal blocking apparatus (FIG. 4) were implanted laparoscopically. Device implantation by the experienced surgeons participating in the study typically took 60 to 90 minutes; five ports were usually used. The electrode itself had an active surface area of 10 mm² and was “c”-shaped to partially encircle the nerve.

Intra-abdominal dissection and electrode placement were accomplished in the following sequence. The gastrohepatic ligament was dissected to expose the esophagogastric junction (EGJ), and the stomach was retracted downward and laterally in order to keep slight tension on the EGJ. To locate the posterior vagal trunk, the right diaphragmatic crus was identified and separated from its esophageal attachments. The anterior vagal trunk was identified by locating it as it courses through the diaphragmatic hiatus. After both vagal trunks had been identified, a right angle grasper was used to dissect a 5 mm window underneath the posterior vagal trunk. The electrode was then placed by positioning a right angle grasper through the window that had been created under the vagal trunk. The electrode's distal suture tab was then grasped, and the electrode was pulled into place, seating the nerve within the electrode cup. The same steps were repeated to place a second electrode around the anterior vagal trunk. Finally, each electrode was secured in position using a single suture placed through each electrode's distal suture tab and affixed to the outer layers of the esophagus.

The leads were then connected to the neuroregulator, and it was implanted in a subcutaneous pocket in the mid-line just below the xiphoid process. Proper electrode placement was then determined in two different ways at implant. First, correct anatomic electrode-nerve alignment was verified visually. Secondly, effective electrical contact was verified using impedance measurements intra-operatively and at frequent intervals thereafter. After recovery from the surgery, a programmable external controller which contained a rechargeable power source was used to communicate transdermally with the implanted neuroregulator via an external transmit coil

Electrical Signal Application

The external controller was programmed for frequency, amplitude and duty cycle. The therapeutic frequency selected to block neural impulses on the vagal trunks was 5000 Hz, based on animal studies of vagal inhibition of pancreatic exocrine secretion. Amplitudes utilized ranged from 1-6 mA; however, in almost all instances, the amplitude was 6 mA. The device was activated in the morning, and turned off before sleep. The protocol specified an algorithm of five minutes of blocking alternating with five minutes without blocking for 12 hours per day. Effective electrical contact was verified using impedance measurements at frequent intervals postoperatively.

Experimental Therapy and Follow-Up Studies

In order to focus on the effects of the vagal blocking apparatus, the study subjects were precluded from receiving either concomitant diet or behavioral counseling or drug therapy for obesity during the 6 month trial period. All study participants were implanted with the device. Two weeks post-implant, intermittent, high-frequency electrical algorithms were commenced in all subjects. Subjects were followed weekly for 4 weeks, then every two weeks until 12 weeks and then monthly visits for body weight, physical examination and adverse event (AE) inquiry. In addition, 12-lead electrocardiograms (ECGs) and clinical chemistries were analyzed at a core laboratory.

Calculation of Percentage Excess Weight Loss

Ideal body weight was calculated by measuring each subject's height and then determining the body weight that would result in a BMI of 25.0 for that subject, i.e., ideal body weight (kg)=25×height² (m). EWL was calculated by dividing weight loss by excess body weight [(total body weight)−(ideal body weight)] and multiplying by 100. Thus, EWL %=(weight loss (kg)/excess body weight (kg))×100.

Data and Statistical Analysis

Baseline characteristics and demographics were summarized using descriptive statistics. Continuous variables were summarized by mean values and corresponding standard errors of the mean (SEM). Categorical (including binary) variables were summarized by frequency distributions.

The primary endpoint for assessing the effect on weight loss was the mean percent excess weight loss (EWL %) at specified time points (4 and 12 weeks and 6 months) and compared to zero in a two-sided, one-sample t-test at the 5% significance level. P-values reported were unadjusted for multiple comparisons. However, the statistical significance was not altered after applying Hochberg's multiple comparison procedure.

Changes in heart rate and blood pressure were summarized over time, using mean and SEM. ECG recordings were collected and analyzed by an independent core lab (Mayo Medical Laboratories, Rochester, Minn., USA). Endpoints included changes in heart rate (HR), PR interval, QRS duration and QTcB interval (QT interval Bazett correction). ECGs were, in all known instances, recorded with the vagal blocking off to detect sustained effects, if any.

Adverse events (AE) were tabulated and reported. No formal statistical analyses of adverse events were performed on the rate of occurrence of adverse events as no a priori hypotheses were specified.

Results

Participants, Demographics and Outcomes of Surgical Procedure

Thirty-nine subjects (mean body mass index 41.2±4.1 kg/m²) received the device. Demographics are shown in Table I.

TABLE I
Demographics of study population (mean ± SEM)

| Demographics | All subjects |
|---|---|
| Number | 39 |
| Age (yrs) | 41.0 ± 9.8 |
| Gender | 33 female/6 male |
| Baseline BMI, kg/m² | 41.2 ± 4.1 |

There have been no major intra-operative complications with implantation of the device. Specifically, we have not encountered organ perforation, significant bleeding, post-operative intra-peritoneal infections, or electrode migration or tissue erosion. The devices were left in place after the 6 month study. Those participants continue to be followed as part of a safety cohort for such a device, and further studies are being conducted to determine whether the electrical parameters can be modified to maximize the efficacy of the device.

Weight Loss

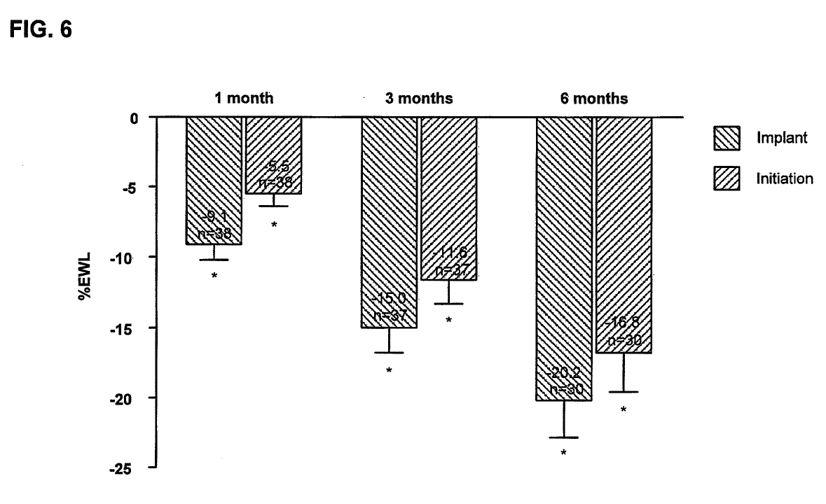

Mean excess weight loss at 4 and 12 weeks and 6 months following device implant was 9.1%, 15% and 20.2%, respectively (all changes were significant compared to baseline, p<0.0001). Beneficial overall effects of treatment were observed at all four centers. FIG. 6 shows the distribution of EWL percentage change. A decrease in waist circumference was also observed. Waist circumference was decreased about 6.4+/−1.4 cm at 3 month and 7.8 cm+/−1.7 cm at six months from a mean baseline of 123.4 cm.

Adverse Events

There were no deaths, no serious adverse events (SAE) related to either the medical device or the electrical signal therapy and no unanticipated adverse device effects during the study. Three subjects, who had SAEs that were unrelated to the device or with vagal blocking therapy, required brief hospitalization: one post-operative lower respiratory tract infection (1 day hospitalization), one subcutaneous implant site seroma (3 days hospitalization), and one case of Clostridium difficile diarrhea two weeks into the trial period (5 days hospitalization). These three SAEs were completely reversible, and the patients continued in the study.

Effects on Heart Rate and Blood Pressure

Patients were also evaluated for changes in heart rate and blood pressure.

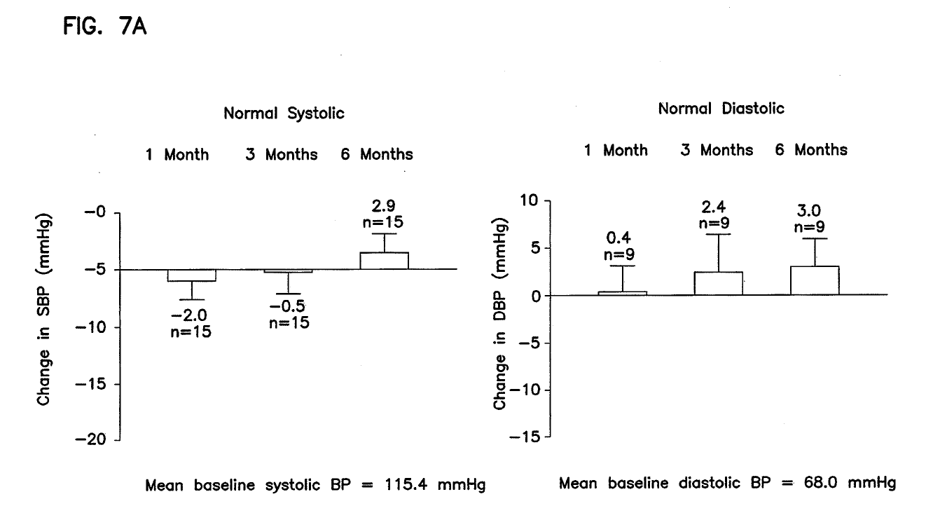

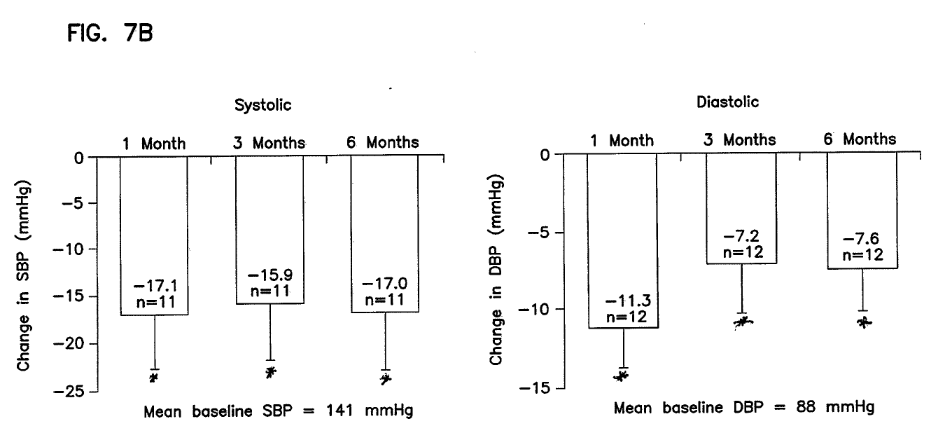

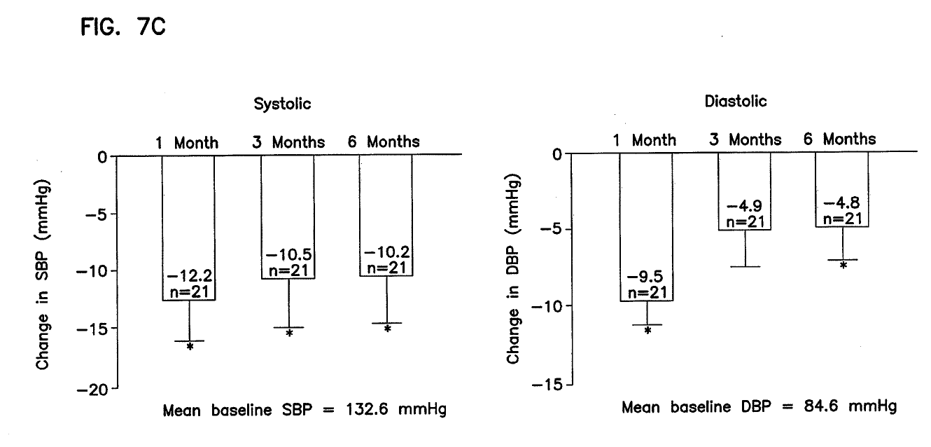

When all of the patients that completed 6 months of treatment were evaluated for changes in blood pressure, about a 10% decrease in systolic and diastolic blood pressure was seen over the 6 month period. (data not shown) Some of the patients had normal blood pressure at the initiation of treatment, these patents did not experience any significant effects on blood pressure. Those patients had a mean baseline systolic pressure of 115.4 mm Hg and a mean baseline diastolic pressure of 68.0 mm Hg. No significant change in blood pressure was observed over the treatment time. See FIG. 7A.

Patients who had elevated blood systolic pressure of greater than or equal to 140 mm Hg and/or diastolic blood pressure greater than equal to 90 mmHg or had a history of hypertension had a mean baseline systolic pressure of 141 mmHg and diastolic pressure of 88 mm Hg before electrical signal treatment. After 6 months of treatment, systolic pressure was decreased 17 mmHg (about 12% decrease) and diastolic pressure was decreased 7.6 mm Hg (about 8.6%) from the mean baseline starting pressures. See FIG. 7B.

Patients who had elevated blood systolic pressure of greater than or equal to 140 mm Hg and/or diastolic blood pressure greater than equal to 90 mmHg and were not diabetic; patients with systolic pressure of greater than or equal to 130 mmHg and/or diastolic pressure of greater than or equal to 80 mm Hg and were diabetic, patients that had a history of hypertension, and patients that had pre-hypertension with a systolic pressure of 120 to 139 mm Hg and/or diastolic pressure of 80 to 90 mm Hg had a mean baseline systolic pressure of 132.6 mmHg and diastolic pressure of 84.6 mm Hg before electrical signal treatment. After 6 months of treatment, systolic pressure was decreased 10.2 mmHg (about 8% decrease) and diastolic pressure was decreased 4.8 mm Hg (about 5.7%) from the mean baseline starting pressures. See FIG. 7C. It should also be noted that patients that had both diabetes and hypertension exhibited significant decreases in systolic and diastolic blood pressure from the mean baseline at the beginning of treatment.(data not shown)

Mean arterial pressure (MAP) in hypertensive subjects also showed reductions at 1, 3, and 6 months. The baseline mean arterial pressure was 101+/−2 mm Hg. At 1 month the MAP was reduced 9+/−3 (p=0.002). At three months, the reduction was 7+/−2 mm Hg (p=0.01). The reduction at 6 months was 6+/−2 (p=0.02).

In another study of hypertension subjects, after 1 week of treatment, significant decreases in systolic pressures, diastolic pressures, and mean arterial pressures were observed. (data not shown)

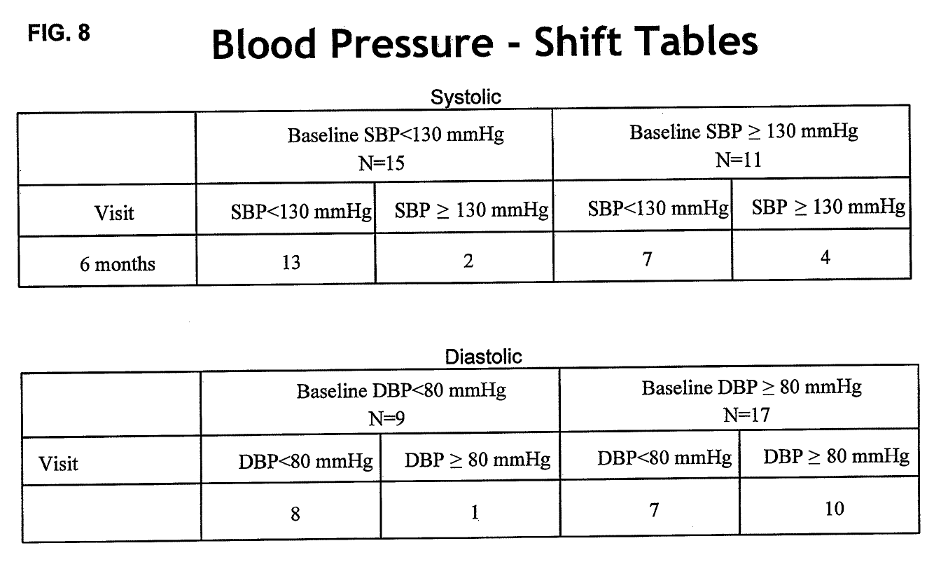

The results showing the shift in systolic and diastolic blood pressure between patients without elevated blood pressure and those with elevated blood pressure at the 6 month visit are shown in FIG. 8. About 70% of the patients having elevated systolic blood pressure saw a drop in systolic blood pressure to below 130 mmHg. About 40% of the patients with elevated diastolic blood pressure showed a drop in diastolic blood pressure to below 80 mmHg. 6 subjects had a concurrent diagnosis of hypertension and were receiving anti-hypertensive medication. 2 of these 6 had reductions in anti-hypertensive meds and a third discontinued all anti-hypertensive meds; in all these instances, blood pressures remained in the normal range.

The results for evaluation of heart rate over 12 weeks of treatment time are shown in Table 2.

TABLE 2

| | | | | | | | |
|---|---|---|---|---|---|---|---|
| | | | Std | Std | | | |
| Visit | N | Mean | dev | err | Min, Max | 95% CI | p-value |

Heart Rate By Visit and Change from Baseline by Visit

| | | | | | | |
|---|---|---|---|---|---|---|
| Base- | 15 | 76.73 | 6.63 | 1.71 | 64, 84 | 73.06, 80.40 |
| line | | | | | | |
| Week 1 | 14 | 73.64 | 12.19 | 3.26 | 54, 101 | 66.61, 80.68 |
| Week 4 | 15 | 70.60 | 11.35 | 2.93 | 52, 93 | 64.31, 76.89 |
| Week | 15 | 69.80 | 9.52 | 2.46 | 53, 85 | 64.53, 75.07 |
| 12 | | | | | | |

Change from BL

| | | | | | | | |
|---|---|---|---|---|---|---|---|
| Week 1 | 14 | −3.00 | 10.40 | 2.78 | −18, 22 | −9.00, 3.00 | 0.30 |
| Week 4 | 15 | −6.13 | 8.05 | 2.08 | −16, 11 | −10.59, −1.67 | 0.01 |
| Week | 15 | −6.93 | 6.36 | 1.64 | −17, 8 | −10.46, −3.41 | 0.0009 |
| 12 | | | | | | | |

To date, 15 of 35 subjects' 12-week ECG data were available for analysis. The results are shown in Tables 3-6.

TABLE 3

| | | | | | | | |
|---|---|---|---|---|---|---|---|
| | | | Std | Std | | | p- |
| Visit | N | Mean | dev | err | Min, Max | 95% CI | value |

PR Interval By Visit and Change from Baseline by Visit

| | | | | | | |
|---|---|---|---|---|---|---|
| Base- | 15 | 164.87 | 22.54 | 5.82 | 118, 211 | 152.39, 177.35 |
| line | | | | | | |
| Week 1 | 14 | 173.57 | 22.53 | 6.02 | 128, 208 | 160.56, 186.58 |
| Week 4 | 15 | 164.27 | 16.92 | 4.37 | 138, 196 | 154.90, 173.64 |
| Week | 15 | 167.33 | 19.67 | 5.08 | 132, 200 | 156.44, 178.23 |
| 12 | | | | | | |

Change from BL

| | | | | | | | |
|---|---|---|---|---|---|---|---|
| Week 1 | 14 | 8.07 | 11.54 | 3.08 | −16, 28 | 1.41, 14.73 | 0.02 |
| Week 4 | 15 | −0.60 | 10.87 | 2.81 | −18, 20 | −6.62, 5.42 | 0.83 |
| Week | 15 | 2.47 | 14.93 | 3.85 | −33, 34 | −5.80, 10.73 | 0.53 |
| 12 | | | | | | | |

TABLE 4

| | | | | | | | |
|---|---|---|---|---|---|---|---|
| | | | Std | Std | | | |
| Visit | N | Mean | dev | err | Min, Max | 95% CI | p-value |

QRS Duration By Visit and Change from Baseline by Visit

| | | | | | | |
|---|---|---|---|---|---|---|
| Baseline | 15 | 91.20 | 9.31 | 2.40 | 80, 108 | 86.04, 96.36 |
| Week 1 | 14 | 92.14 | 9.92 | 2.65 | 76, 114 | 86.41, 97.87 |
| Week 4 | 15 | 90.53 | 9.13 | 2.36 | 74, 112 | 85.48, 95.59 |
| Week 12 | 15 | 91.33 | 9.49 | 2.45 | 74, 108 | 86.08, 96.59 |

Change from BL

| | | | | | | | |
|---|---|---|---|---|---|---|---|
| Week 1 | 14 | 0.43 | 4.48 | 1.20 | −6, 10 | −2.16, 3.02 | 0.73 |
| Week 4 | 15 | −0.67 | 4.86 | 1.26 | −8, 8 | −3.36, 2.03 | 0.60 |
| Week 12 | 15 | 0.13 | 7.26 | 1.87 | −9, 22 | −3.89, 4.15 | 0.94 |

TABLE 5

| | | | | | | | |
|---|---|---|---|---|---|---|---|
| | | | Std | Std | | | p- |
| Visit | N | Mean | dev | err | Min, Max | 95% CI | value |

QT Interval By Visit and Change from Baseline by Visit

| | | | | | | |
|---|---|---|---|---|---|---|
| Base- | 15 | 380.27 | 23.89 | 6.17 | 352, 435 | 367.04, 393.50 |
| line | | | | | | |
| Week 1 | 14 | 378.93 | 29.66 | 7.93 | 323, 441 | 361.80, 396.05 |
| Week 4 | 15 | 387.53 | 26.16 | 6.75 | 350, 443 | 373.05, 402.02 |
| Week | 15 | 389.80 | 25.76 | 6.65 | 356, 441 | 375.53, 404.07 |
| 12 | | | | | | |

Change from BL

| | | | | | | | |
|---|---|---|---|---|---|---|---|
| Week 1 | 14 | −0.79 | 18.06 | 4.83 | −36, 37 | −11.21, 9.64 | 0.87 |
| Week 4 | 15 | 7.27 | 22.26 | 5.75 | −38, 43 | −5.06, 19.59 | 0.23 |
| Week | 15 | 9.53 | 13.73 | 3.54 | −12, 44 | 1.93, 17.13 | 0.02 |
| 12 | | | | | | | |

TABLE 6

| | | | | | | | |
|---|---|---|---|---|---|---|---|
| | | | Std | Std | | | p- |
| Visit | N | Mean | dev | err | Min, Max | 95% CI | value |

QTc Bazett By Visit and Change from Baseline by Visit

| | | | | | | |
|---|---|---|---|---|---|---|
| Base- | 15 | 428.73 | 19.95 | 5.15 | 398, 469 | 417.68, 439.78 |
| line | | | | | | |
| Week 1 | 14 | 416.14 | 17.50 | 4.68 | 381, 445 | 406.04, 426.25 |
| Week 4 | 15 | 417.33 | 22.46 | 5.80 | 393, 465 | 404.90, 429.77 |
| Week | 15 | 417.87 | 18.34 | 4.73 | 389, 456 | 407.71, 428.02 |
| 12 | | | | | | |

Change from BL

| | | | | | | | |
|---|---|---|---|---|---|---|---|
| Week 1 | 14 | −11.64 | 25.82 | 6.90 | −88, 21 | −26.55, 3.27 | 0.12 |
| Week 4 | 15 | −11.40 | 23.14 | 5.97 | −43, 44 | −24.21, 1.41 | 0.08 |
| Week | 15 | −10.87 | 19.34 | 4.99 | −39, 35 | −21.58, −0.16 | 0.047 |
| 12 | | | | | | | |

Compared with baseline, HR decreased a mean 6.9 bpm (p<0.001), consistent with observed weight loss. Mean PR interval and QRS duration were unchanged (+2.5 msec, p=0.53 and +0.13 msec, p=0.94, respectively). Mean QTcB changed −10.9 msec (p=0.05), consistent with HR changes and not deemed clinically significant.

Discussion

In this clinical trial of an implantable apparatus that delivers intermittent vagal blocking (electrical signal therapy), we report here on safety and efficacy—as measured by EWL %. The % EWL shows that the patients had 20% EWL after 6 months of treatment. In addition, the sub-studies conducted have shown that the weight loss is associated with decreased blood pressure in patients with elevated blood pressure.

Weight reduction observed in this study was progressive out to 6 months of follow-up without an apparent plateau. It is important to note that this effect on weight was achieved without the additional benefit of dietary or behavioral modification, which may augment weight reduction with any intervention. While we cannot completely exclude a placebo effect, given the open trial design, we expect that this is unlikely since the reduced caloric intake, time to satiation at meals and hunger between meals were achieved early after onset of treatment, were maintained throughout the 6 month study, and were associated with significant and sustained weight loss.

Safety of the novel device and electrical signal applied as described herein is supported by the fact that the only notable complications were three infections related to the surgical procedure or C. difficile diarrhea, all of which were considered by an independent data safety monitoring committee to be unrelated to the device itself. There were no major intra-operative complications. Specifically, we did not encounter organ perforation or significant bleeding. Furthermore, we did not observe post-operative intra-peritoneal infections, electrode migration or tissue erosion.

The present studies provide some insights on the mechanism for the weight loss associated with electrical signal therapy. The vagus nerve has pivotal roles in multiple aspects of organ function. Changes in cardiovascular parameters such as decreases in blood pressure and heart rate for those patients that have elevated blood pressure are in further support of the efficacy and safety of this treatment. Patients without hypertension or without prehypertension did not have any significant change in blood pressure over the treatment period. Although the current sample size is small, the effects on blood pressure and heart rate are important to note since the vagus is a prominent regulator of parasympathetic tone on the cardiovascular apparatus at the thoracic level. The intermittent vagal blockade is applied at the sub-diaphragmatic level and is effective to reduce blood pressure without adversely affecting other cardiac functions as evidenced by the ECG parameters or without other side effects. In some cases, the treatment was effective to normalize blood pressure and allow patients to discontinue drug treatment. In other cases, the treatment provided for a reduction in the medication that the patients were taking.

Based on the findings from this clinical trial, it can be concluded that intermittent, intra-abdominal vagal blocking using a novel, programmable medical device is associated with both significant excess weight loss and a desirable safety profile. Furthermore, study data support the therapeutic rationale of intermittent, intra-abdominal vagal blocking for treatment of hypertension, congestive heart failure, and/or other conditions that have hypertension as a component.

### Example 2

### Materials and Methods

### Study Design

This study was a prospective, open-label, multi-center study to evaluate the safety and efficacy of high frequency electrical algorithms applied to the intra-abdominal vagal trunks in facilitating weight loss and improving glycemic control and blood pressure in type 2 diabetics. Subject's pre-implant baseline measurements served as the control.

This study was conducted at Instituto National de la Nutricion (INNSZ), Mexico City, Mexico; Trondheim University Hospital, Trondheim, Norway; University Hospital, Basel, Switzerland; Flinders Medical Centre, Adelaide, Australia; and Institute of Weight Control, Sydney, Australia. The study was registered on “clinicaltrials.gov” (NCT00555958).

### Study Subjects

Device safety and efficacy were assessed during a 12-month study in obese female and male subjects (body mass index (BMI) 30-40 kg/m² inclusive, age 25-60 years inclusive) with type 2 diabetes. Written informed consent was provided from all subjects. The study was approved by local medical ethics committees. General inclusion criteria included prior failure of durable response to medical weight management that involved diet, behavioral modification and/or pharmacotherapy. Fertile women required contraception and proof of non-pregnancy within 14 days of implant. Relevant exclusion criteria included type 1 diabetes mellitus, smoking cessation within 6 months and weight loss drug therapy within the last 3 months, significant weight loss in the last 12 months (>10% body weight loss), hiatal hernia, an implanted electrical medical device or major abdominal surgery, excluding cholecystectomy and hysterectomy. Inclusion criteria included Type 2 diabetes ≦12 years duration of diabetes, baseline HbA_{1c} levels ≧7% to ≦10% and absence of significant type 2 diabetes complications, such as nephropathy, retinopathy, neuropathy or coronary artery disease. Diabetes-related exclusion criteria included insulin dependence and use of GLP-1 receptor agonists. Short-term insulin use was allowed during the peri-operative period if needed.

### Study Device and Implantation Method

Subjects received a fully implantable Maestro Apparatus (Maestro RC2 Apparatus) consisting of two leads, placed laparoscopically as previously described,¹ one on each intra-abdominal vagal trunk connected to a subcutaneously implanted, rechargeable neuroregulator. A mobile charger was used to recharge the device battery, most commonly for 30 minutes daily.

### Therapy and Follow-Up Studies

Devices were activated approximately two weeks post-implantation. Biphasic pulses at a frequency of 5000 Hz and amplitude from 3 to 8 mA (mode=6) were applied to block vagal neural impulses, with a duty cycle of 5 minutes blocked then 5 minutes unblocked for up to 15 hours daily. The objective was for patients to receive a minimum of 12 hours to a maximum of 15 hours therapy daily depending on patient's reaction to therapy and daily lifestyle.

All subjects received 15 individual weight management counseling sessions during which basic weight loss and physical activity information was delivered. The initial session is 45 minutes, sessions 2-4 are 30 minutes and the remaining sessions are 15 minutes long. Only standard weight management materials were used. No support groups, behavioral therapists or exercise specialists were employed in this trial. General information regarding weight loss, calorie goals, healthy eating strategies, exercise strategies and record keeping are discussed.

Weight was measured at baseline, weekly through 4 weeks, biweekly to 12 weeks and monthly to 12 months. HbA_{1c} and fasting plasma glucose (FPG) were measured (ICON Laboratories, Farmingdale, N.Y.) at baseline, 1 week, 4 weeks, 12 weeks and 6 and 12 months. Blood pressure was measured in triplicate, with subjects seated, at 5 minute intervals between measurements using a properly sized cuff (i.e., standard adult size (16×30 cm) for arm circumference of 27 to 34 cm or large adult size (16×36 cm) for 35 to 44 cm arm circumference) at baseline, 1 week, 4 weeks, 12 weeks and 6 and 12 months. Hypertension was defined as systolic blood pressures ≧130 mmHg and/or diastolic blood pressures >80 mmHg according to the JNC-7 criteria for type 2 diabetics.¹⁴ Waist circumference was measured at the iliac crest (NHANES III Protocol).

Adverse event (AE) inquiries, clinical laboratory assessments and 12-lead electrocardiogram findings (Mayo Medical Laboratories, Rochester, Minn. and Quintiles Limited, Berkshire, England) were completed at each visit. Medication changes and dose adjustments were recorded at each visit. Neither the surgeon nor the allied health professional from the clinic were involved in any treatment decisions to reduce or cease any medication.

### Calculation of Percent EWL

Ideal body weight was determined by measuring each subject's height and calculating the body weight at BMI of 25.0 for that subject (i.e., ideal body weight (kg) =25×height (m)²). Next, excess body weight in kg (total body weight at baseline−ideal body weight) was determined and percent EWL was calculated (weight loss/excess body weight×100).

### Statistical Analysis

Baseline characteristics and demographics were summarized using descriptive statistics. Mean values with standard errors of the mean (SEM) summarized continuous variables while frequency distributions were summarized as categorical (including binary) variables. Mean excess weight loss (EWL %) and changes in HbA_{1c}, FPG and blood pressure (mean arterial pressure, systolic blood pressure and diastolic blood pressure) at 1, 4 and 12 weeks and 6 and 12 months were assessed using two-sided, one sample t-tests. Changes in waist circumference at 12 weeks and 12 months were assessed using a two-sided, one sample t-test. The rate of occurrence of AEs was analyzed.

### Results

### Participants and Demographics

A total of 28 qualifying subjects were enrolled (17 females and 11 males; mean age 50.9±8.6 years; mean BMI 37.0±3.3 kg/m²). Twenty six subjects completed 12 months of follow-up, whose demographics were 11 males and 17 females, mean age of 50.9±8.6 years and BMI of 37.3±3.3 kg/m. Two of the subjects did not attend the 12 month visit, but did not drop out. No subjects have discontinued the trial and all subjects continue to be followed to assess safety and efficacy.

### Safety

All procedures were successful laparoscopically. There were no complications and all patients were discharged either the same day or following day as consistent with normal hospital policy. There were no deaths or operative complications. In addition there were no unanticipated adverse device effects. One serious adverse event (SAE) occurred in this trial. The SAE was implant site pain as a result of neuroregulator placement directly over the ribs. The discomfort was eliminated by moving the neuroregulator inferior to the costal margin in the left loin. All measured blood tests and electrocardiograms were normal throughout the study.

### Weight Loss

Percent EWL was noted immediately following device activation (data not shown). Average hours of therapy delivery per day over the 12 months were 14.1±0.1 hours with 6.2±0.1 mA average current amplitude. A 24.5% excess weight loss was observed at 6 and 12 months of therapy. (data not shown)

### Changes in Glycemic Control

HbA_{1c} was reduced from a baseline of 7.8±0.2% (mean±SEM). FPG reduction was from a mean baseline of 151.4±34.2 mg/dL. A decrease of 1% of HbA_{1c} was seen in patients at the 6 and 12 month time periods. Fasting plasma glucose was decreased about 28 mg/dL. (data not shown)

At initiation, twelve subjects took one diabetes medication and 6 subjects took two diabetes medications. By the 12 month visit, two subjects discontinued their diabetes medication, six subjects decreased the dose of medications while twelve subjects had no change (84% overall maintained or decreased medication). Four subjects increased diabetes medications.

### Change in Blood Pressure

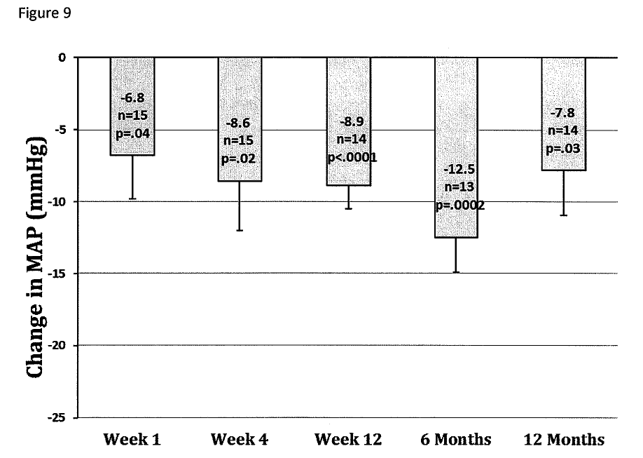

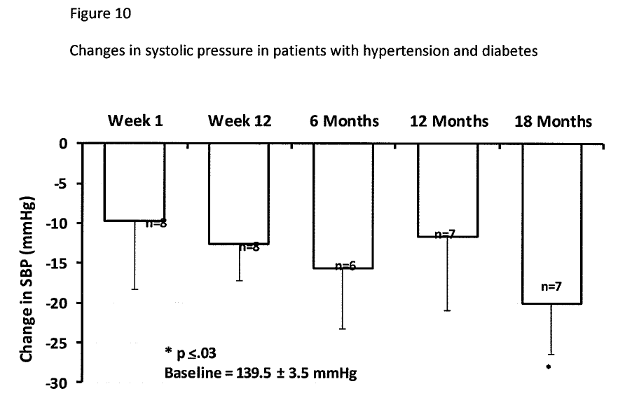

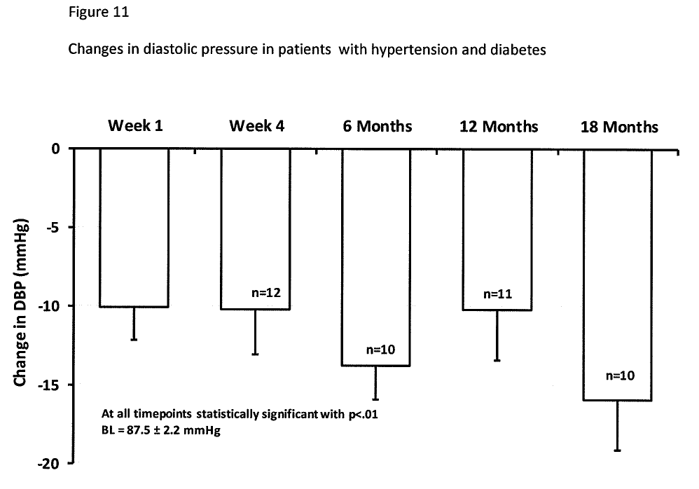

Hypertension (SBP≧130 and/or DBP>80 mmHg) was documented in 15 of the obese diabetic subjects. FIG. 9 shows significantly reduced mean arterial blood pressure (MAP) in subjects with elevated systolic and/or diastolic blood pressure to non-hypertensive levels in all cases from a baseline of 100.1±2.4 mmHg at all time points. Significant reductions were also observed in subjects with elevated SPB at the 18 month time point. FIG. 11 shows SPB reductions from a baseline of 139.5±3.5 mmHg (n=8). Significant reductions were observed in subjects with elevated DBP at all time points from a baseline of 87.5±2.2 mmHg, n=12. FIG. 10.

Five subjects took one medication for hypertension and one subject took two medications at baseline. One subject reduced hypertension medications, four subjects were unchanged and one subject increased medication during the trial. Importantly, the therapy did not significantly change blood pressure in subjects with normal preoperative MAP (data not shown).

Discussion

This open label prospective trial of VBLOC therapy in obese type 2 diabetic patients demonstrated that VBLOC therapy was safe and effective for achieving clinically significant weight loss and improving both T2DM and hypertension. Additionally, there were no untoward events and the therapy was well tolerated by nearly all of the patients.

The ramifications of the increase in the incidence and prevalence of obesity and T2DM in the United States and throughout the world are becoming well understood as they affect both budgets and the public health of nations. Currently, over two thirds of Americans are overweight and over one quarter are obese. In addition, approximately 8% of U.S. adults and 19% of adults over 65 years of age are diabetic. Even more sobering is that fact that the coexistence of type 2 diabetes and obesity increases the risk of developing hypertension and cardiovascular disease thereby increasing morbidity and mortality. There is also good reason to believe that the prevalence of these conditions will continue to increase around the globe. The cost to provide medical care for obesity and T2DM are projected to be unsustainable.

While current bariatric surgical procedures have been shown, to various degrees, to be highly successful for improving (and even forcing into remission) these devastating chronic illnesses, too few candidates undergo these operative procedures. This disconnect between an efficacious treatment and potential candidates for it is multifactorial. It includes factors such as insurance access restrictions, prejudices against the obese, and the fear of the perioperative risks and long term consequences of these procedures. In short, it is clear that for most obese patients, conventional bariatric surgery is not a viable option. This phenomenon has created a significant need for new and novel interventions that are safer, effective for both weight control and T2DM and offer fewer long-term health and life-style consequences.

One such new technology is vagal nerve activity blocking with a patterned electrical impulse delivered to the intra-abdominal nerve trunks. Based on the growing understanding of the vagus nerve in energy regulation, appetite, and glucose regulation, VBLOC is increasingly showing itself to be safe and effective. In this trial, the therapy was studied in a cohort of obese patients (mean BMI 37.0±3.3 kg/m²) with T2DM and hypertension. Clinically significant weight loss of 24.5% EWL occurred by 12 months. Early improvements in glycemic control were observed. HbA_{1c} levels were reduced to 7.1% from a baseline of 7.8% by 4 weeks and fell to 6.9% by 12 weeks. This reduction was maintained at 12 months. Twenty one of 25 subjects (84%) were found to be able to maintain, decrease or discontinue their diabetes medications during the first 12 months while achieving improved glucose control. Improvements in blood pressure were also observed in the hypertensive subjects with no adverse changes in normotensive subjects. Five of six subjects (83%) maintained or decreased hypertensive medications while achieving improved blood pressure control.

The addition of VBLOC therapy to an existing medication regimen resulted in significant improvements in glucose regulation in the T2DM cohort and blood pressure control in the hypertensive cohort, while allowing 80%+of subjects to reduce or maintain their medication. All medication decisions were made by the patient's primary physician and not by the investigators and, for example, some diabetic patients remained on metformin for the cardiovascular protective effects despite improved blood sugars.

### EXAMPLE 3

This study was a multicenter, prospective, randomized, double-blind, controlled, parallel group trial with a 12-month post-randomization follow-up period. All subjects in both groups received all of the implantable components of the Maestro Apparatus® (EnteroMedics Inc, St. Paul, Minn.) at the time of implantation. Non-diabetic, obese subjects were randomized in a 2:1 allocation to the treated group and control group at the time of initiating therapy. A limited number of type 2 diabetics were randomized in a 1:1 allocation. At the end of the blinded, 1 year follow-up period, all subjects received open-label VBLOC Therapy and are continuing to be followed for an additional 4 years.

### Study Centers

Fifteen academic and/or private practice clinical sites participated in the EMPOWER study (see list of contributing centers). All surgeons had been involved in either VBLOC feasibility studies or underwent training in the classroom and animal laboratory on placement of the Maestro Apparatus® under supervision by a laparoscopic surgeon experienced with the technique of placement. Surgeons experienced with implanting the Maestro Apparatus also performed on-site proctoring. The FDA and the respective institutional review board at each center approved the protocol followed by the centers.

### Study Population

Subjects seeking weight loss surgery at the clinical sites composed the study subjects. The main criteria for inclusion were consistent with the 1992 NIH guidelines for bariatric surgery and included male or female obese subjects 18 to 65 y of age inclusive with a body mass index (BMI) 40 to 45 kg/m² or 35 to 39.9 kg/m² with one or more of the following obesity-related, co-morbid conditions: hypertension defined by a blood pressure ≧140/90 mmHg or pharmacologically treated hypertension (with blood pressure ≦140/90 mmHg), dyslipidemia defined by total cholesterol ≧200 mg/dL or LDL ≧130 mg/dL or pharmacologically treated dyslipidemia (with total cholesterol <200 or LDL <130 mg/dL), documented sleep apnea, type 2 diabetes (defined as HbA1c ≧6.5-9%, onset of ≦10 y, stable treatment in last 3 mo, currently not using the following: insulin, GLP-1 receptor agonists or dipeptidyl peptidase (DPP-4) inhibitors for the last 6 mo, and creatinine within normal range and no history of retinopathy, neuropathy, cardiovascular or vascular disease), or obesity-related cardiomyopathy (defined as an ejection fraction <40% on echocardiography). Written informed consent was obtained to participate in the study.

All subjects had not achieved satisfactory or sustained weight loss with diet, behavioral intervention, and/or pharmacotherapy. Females of child-bearing potential had a negative urine pregnancy test both at study entry and within 14 days of the implant procedure followed by commitment to follow their physician-approved contraceptive regimen for the full study period. Ability to complete all study visits and procedures was an eligibility requirement. Relevant exclusion criteria included: type 1 diabetes mellitus (DM) or type 2 DM poorly controlled with oral hypoglycemic agents or with associated autonomic neuropathy and/or gastroparesis; treatment with pharmacologic weight-loss therapy; smoking cessation within the prior 3 mo; decrease of more than 10% of body weight in the previous 12 mo; prior gastric resection or other major abdominal surgery excluding cholecystectomy and hysterectomy; clinically important hiatal hernia or intra-operatively determined hiatal hernia requiring operative repair or extensive dissection at the esophagogastric junction at time of operation for potential electrode implantation; and presence of a permanently implanted, electrical powered medical device or implanted gastrointestinal device or prosthesis. Concurrent treatment for thyroid disorders, epilepsy, or depression with tricyclic agents was acceptable for participation if the treatment regimen was stable for the prior 6 mo.

### Operative Technique of Electrode Placement

The device was implanted as described previously in Example 1.

### Device Activation, Randomization Assignment, and Electrical Algorithms

The subjects attended a visit for randomization and device activation 7 to 21 days after implantation of the Maestro Apparatus® and were randomized to treated or control in a blinded manner. Randomization for non-diabetics was conducted in a randomized permuted block design (block sizes of three or six), stratified by investigational center. Neither the subject, nor the study follow-up team, nor the sponsor knew the treatment assignment.

The external controller was programmed for frequency, amplitude, and duty cycle. Biphasic pulses at a frequency of 5000 Hz and amplitude from 3 to 8 mA (mode=6 mA) were applied to block vagal neural impulses completely in the treated group only; this blockade was accomplished with a monotonous duty cycle of 5 min of 5000 Hz of electrical vagal blockade, then 5 min of no electrical signal (unblocked); this duty cycle of 5 min ON followed by 5 min OFF (with no impulse delivered) continued for the duration of time while the external controller was worn.

Subjects in the control group also received electrical impulses during the ON cycle consisting of two bursts of 13 impulses at 1000 Hz and 3 mA of 26 msec duration at both time 0 and time 3 min of the ON cycle and 40 Hz up to 1 mA stimulation throughout the duration of the 5 min ON cycle. This control algorithm was performed during the entire 5 min of the ON period to ensure good working order and safety of the device and to facilitate blinding of the study. Note, the Maestro Apparatus® in the control group was fully operational in order to assure that the device could be fully activated at one year when the main body of the study was completed; the control subjects were recruited with the understanding that after one year, the device would be activated for the next 4 y. Also, the apparatus had to check itself in order to determine the amount of time the external components were used. Treated subjects also received impedance checks and safety checks at the start of every therapy cycle. If the device was worn for 10 h/day, the total charge delivered to the treated and control groups was 3.9 vs 0.0014 Coulombs, respectively. The charge delivered to the vagus nerves in the control group had been determined to be low and based on prior acute animal electrophysiology testing, this degree of electrical input was assumed to be of no long term clinical or physiologic importance.

All subjects were encouraged to use the device for a minimum of 9 h per day and up to 16 h daily. Because the controller and power source required the subject to be compliant with wearing the components to receive therapy, hours of therapy delivery were ultimately under the control of the subject. By design, the device recorded the hours per day the controller was actually worn. The subject was instructed to wear the external components after bathing or showering in the morning and to take them off before sleep.

All subjects received 15 individual counseling sessions of weight management, where basic information on weight loss and physical activity was delivered and discussed. Materials to document diet and exercise were provided. No preoperative psychological testing or interviews were conducted.

The primary effectiveness objective was to demonstrate a significantly greater percentage of excess weight loss (% EWL) in the treated group compared to the non-treated control group at 12 mo using a statistical, super-superiority test margin of 10%. % EWL was calculated as the difference in implantation and postoperative weights divided by the difference in the implantation weight and ideal body weight using the BMI method; a BMI of 25 was considered ideal. Subjects were weighed on the same calibrated, electronic scale throughout the study. Weight was measured at implantation, weekly through wk 4, and monthly to 12 mo for the first year of the study.

The secondary effectiveness objective was to determine if a significantly greater percent of subjects in the treated group achieved 25% EWL compared to control subjects. The safety objectives were to estimate the rate of serious adverse events (SAEs) related to the implant procedure, the device, or the VBLOC Therapy delivered by the Maestro Apparatus®. Inquiries concerning adverse events (AE) were completed at each visit. A 12-lead electrocardiogram was obtained at baseline and at 4 wk and 6 and 12 mo post-activation, and 24-h Holter monitor testing was conducted at screening and at 3, 6, and 12 mo post-activation. Readings of the ECG and Holter tests were performed by a central laboratory (Duke University, North Carolina). Medication changes and dose adjustments were recorded at each visit.

Other assessments included clinical laboratory measures at screening, implantation, device activation, and at 4 wk and 6 and 12 mo after activation of the device. All laboratory tests were performed by a central laboratory (ICON Laboratories, Farmingdale, N.Y.). Vital signs (blood pressure, pulse, and temperature) were measured at all visits. Hypertension was defined as systolic blood pressure ≧140 mmHg and/or diastolic blood pressures >90 mmHg according to the JNC-7 criteria for adults. Subject questionnaires were conducted at screening and 6 and 12 mo after activation, including hunger and appetite assessment via a hunger and appetite 100 mm visual analogue scale questionnaire, Three Factor Eating Questionnaire, quality of life (SF-36®), and the Impact of Weight on Quality of Life questionnaire, and depression assessed by the Beck Depression Inventory, BDI®-II.

### Statistical Analysis

Sample size was calculated using Statistical Analysis System Version 9.2 software (Proc Power, SAS Institute, Cary N.C.) to compare two means. The minimum required sample size was calculated under the following assumption: significance level=2.5%, power=90%; the expected % EWL in the OFF group=8%, the expected % EWL in the ON group=25%, and the expected standard deviation=15% (VBLOC feasibility trials). Under the assumptions outlined above, the estimated minimum sample size was 222 subjects. The study enrolled 294 subjects anticipating 23% attrition in both groups (excluding the first 14 surgical subjects implanted at centers who had never previously implanted a vagal blockade device in a human). The primary analysis was conducted according to the principals of intent-to-treat. All subjects were analyzed according to randomization assignment. The primary analysis compared 12 mo results across treatment groups, comparing the observed difference to the null value of 10% any missing data were assumed to be “missing at random.” Supportive mixed model, repeated measures regression analyses (SAS Prox Mixed) were conducted using all data available, and modeling any missing data. A sensitivity analysis was also performed which applied the “last value carried forward” imputation method to any missing 12-mo data points. Continuous data are presented as mean ±standard error of the mean ( x̅±SEM).

Results

Subjects Enrolled

After enrolling 503 subjects, 299 were excluded for failure to meet inclusion criteria despite screening, lack of confidence by the investigator team in subject compliance, subject decision, etc. A total of 294 subjects were implanted with the Maestro Apparatus® and were randomized to the treated group (n=192) or the non-treated control group (n=102). First implantations for respective surgeons who had not placed an apparatus previously were not evaluated in the primary or secondary safety or efficacy endpoints by the original design of the study per FDA agreement. The treated analysis group consisted of 18 males (10%) and 165 females (90%) with age=46±1 years and a BMI of 41±1 kg/m². There were 5 treated subjects (3%) with Type 2 diabetes mellitus. The control group had 14 males (14%) and 83 females (86%) with age of 46±1 years and BMI of 41±1 kg/m²; 5 (5%) had Type 2 diabetes mellitus.

There were 14 subjects in the treated group (7%) and 5 in the control group (5%) who withdrew prior to completing the 12-mo trial. Reasons for withdrawal in the treated and control groups, respectively, included an adverse event in 4% and 1%, loss to follow-up in 1% each, and personal decision in 3% each.

### Safety

There were no deaths or unanticipated adverse device effects (UADEs). There were 35 serious adverse events (data not shown). The DSMB determined these SAEs to be related to a pre-existing condition (17), the operative procedure/anesthesia (4), the implantation or revision of the device (5), the device (4), the therapy algorithm (0), or to be unrelated to any of these (5). One subject developed bronchospasm on induction of anesthesia, and the operation was cancelled; the implantation was not performed, and the subject was not randomized. None of the implantation SAEs was life-threatening, required emergency operation, or necessitated removing the subject from the study. Three subjects developed infection at the site of the neuroregulator requiring either successful antibiotic treatment alone (n=2) or in one subject, removal of the device due to the presence of purulent fluid. Sixteen subjects wanted the device removed (8 for an adverse event, 8 for subject decision), and 14 subjects required a revisionary procedure to make the device operational or for an adverse event (three for pain at the neuroregulator site and two for high lead impedance, 8 for problems with neuroregulator communication, and 1 for neuroregulator location interfering with coil placement). No subject in either group developed abnormalities in their ECG, such as abnormalities in the PR interval, QRS duration, or the ventricular repolarization interval (QTcF interval), and no abnormalities were noted with Holter monitoring.

### Efficacy—Weight Loss

When comparing the treated group with the control group at the 12-month evaluation, there was no difference in overall weight loss measured as % EWL (17±2 vs. 16±2, p=NS). Similarly, the percentage of subjects attaining a weight loss of ≧25% EWL was also not different between groups (22% vs. 25%, p=NS).

### Subgroup Analyses

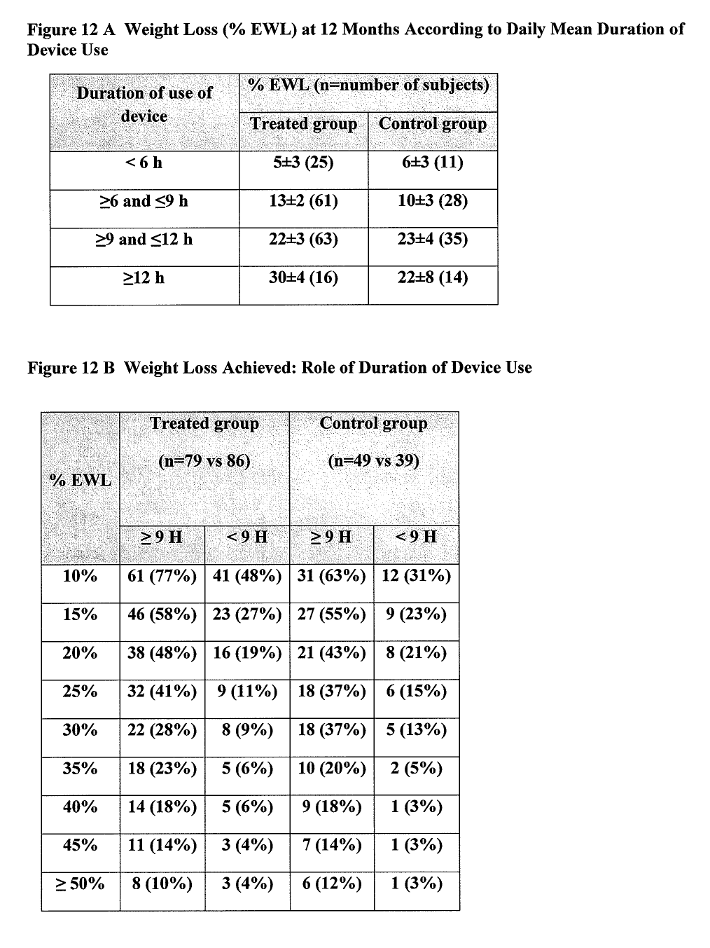

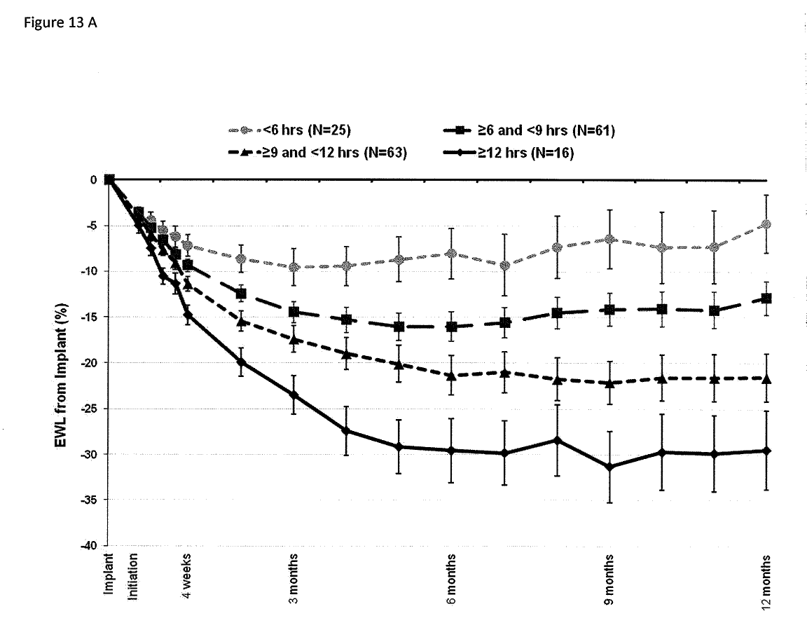

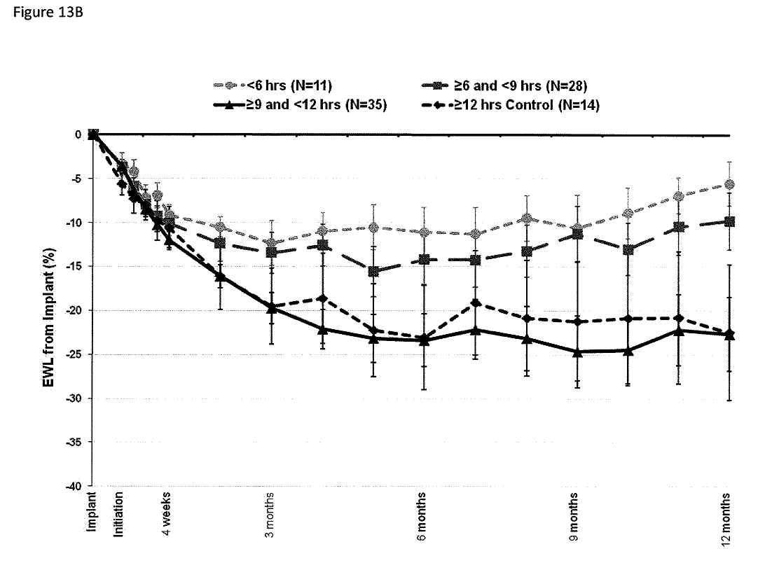

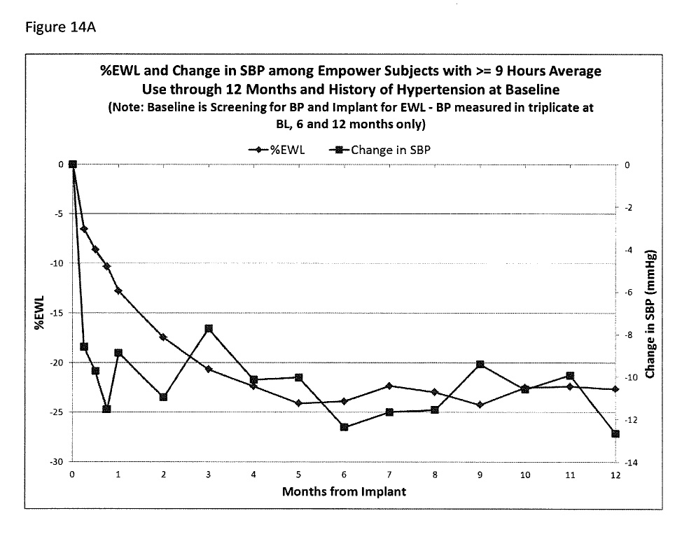

Weight loss by hours of use/day: There were no differences between groups in compliance with device usage defined as hours of device use per day. There was, however, a strong and statistically significant association (repeated measures regression analysis; p<0.001) with improved % EWL from baseline weight with greater hours of device use per day regardless of treatment group (FIGS. 12A-B, FIG. 13A-B). When the device was used for ≧12 h/day, % EWL was 30±4 in the treated group (n=16) and 22±8, respectively in the control group (n=14, p=0.42). %TBWL(total body weight lost) was 11.4±1.7 in the treated group and 8.3±3.0 in the control group.

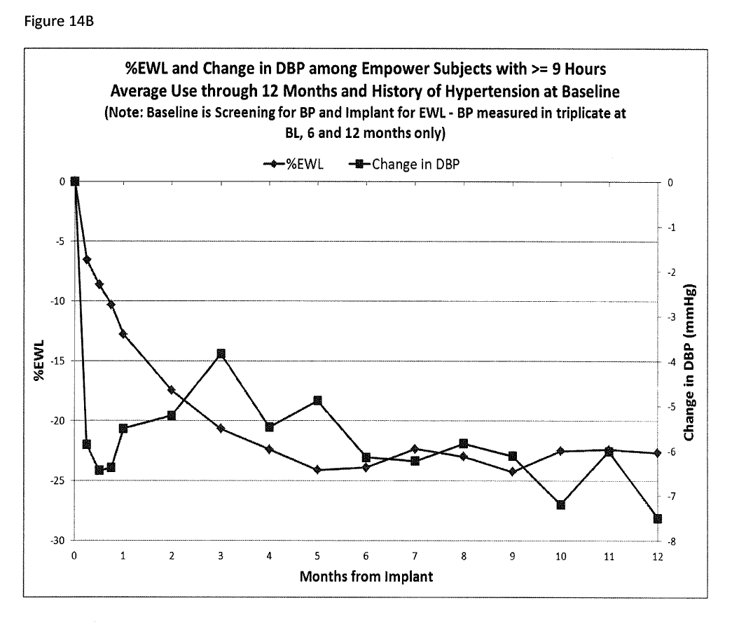

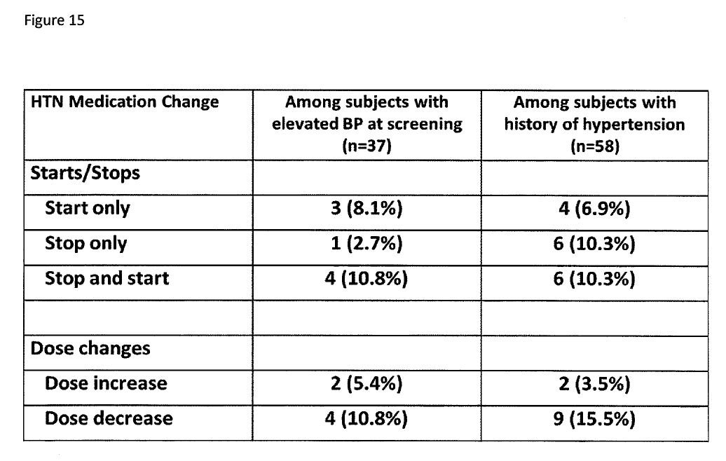

Effects on Blood Pressure: In both groups, subjects with a medical history of hypertension at entry into the study (n=77 or 42% in treated group, n=40 or 41% in control group) had improvements in blood pressure (p<0.01) as measured by changes in systolic blood pressure at 6 mo (−10±2 vs. −9±3 mmHg) and 12 mo (−10±2 vs −9±3 mmHg) from a baseline of 133 mmHg (for both treated and control) and diastolic blood pressure at 6 mo (−4±1 vs. −8±2 mmHg) and 12 mo (−5±1 and −5±2 mmHg) from a baseline of 83 mmHg (for both treated and control), respectively. FIG. 14A-B. No differences, however, were noted between study groups. Subjects without hypertension at baseline had no meaningful changes in blood pressure at some time points (data not shown). The studies were also analyzed for the effect on changes in hypertensive medications including stoppoing medications or changing medications. FIG. 15.

### Discussion

Long established data establishes that intermittent, reversible blockade of the vagus nerve produces weight loss and modulates gut function and serves as a sensory pathway from the gut to the brain. The EMPOWER study was designed to evaluate the effects of intermittent, bilateral blockade of both subdiaphragmatic vagal nerves to induce a feeling of satiety, decrease food intake, and to cause and maintain a clinically relevant weight loss in subjects with morbid obesity. Preliminary work in a trial of intermittent vagal blockade (VBLOC study) suggested that this approach was promising. Subjects in the VBLOC study lost 23% EWL after 6 mo of intermittent vagal blockade. The current EMPOWER study was designed specifically as a randomized, double-blinded, multicenter, controlled trial of intermittent vagal blockade in subjects with morbid obesity to verify the VBLOC study.

The primary effectiveness objective of EMPOWER was to demonstrate a difference in % EWL between the treated group and the control group. At 1 year of treatment, % EWL was virtually identical between groups (treated: 17±2% vs control: 16±2%, p=NS). The secondary effectiveness objective, to determine if more subjects in the treated group vs. the control group achieved >25% EWL, was also not achieved, with 22% in the treated group and 25% in the control group achieving 25% EWL (p=NS). Therefore, under the experimental design and conditions of vagal blockade of this EMPOWER study, no statistical or clinically relevant differences in weight loss were noted between the treated and control groups.

Important differences in weight loss were noted in both groups. First, the mean % EWL in both groups was greater than the expected % EWL of approximately 8% with lifestyle intervention alone. Second, when subjects in each group were divided according to mean duration of vagal blockade per day, the weight loss was greater with increasing use in both groups (p<0.001, repeated measures analysis). FIGS. 13A and B. Those subjects who wore the vagal blockade device routinely for ≧12 h/day in both groups (treated group: 16 subjects; control group: 14 subjects) lost 30±4% and 22±8%, respectively, while those who wore the device >6 but ≦9 h/day (treated group: 61 subjects; control group: 28 subjects) lost only 13±2% and 10±3%. Furthermore, satiety increased and hunger decreased in both groups, again suggesting an effect of the device. FIG. 12A.

Important differences were also noted in blood pressure for those subjects that were hypertensive at baseline(>140/90 for non-diabetics or >130/80 for diabetics; N=37). In subjects with ≧9 hrs of device use over 12 months as compared to subjects with Hypertension History at Baseline (N=58) both systolic and diastolic BP was substantially reduced by 2 weeks post-screening prior to any substantial weight loss in both populations. Subjects with ≧9 hrs of device use over 12 months saw a decrease of SBP of 17-18 mmHg and a decrease in DBP of 9-10 mmHg. FIG. 14A-B. In the group of hypertension at baseline, a decrease in SBP of 10-13 mmHg and DBP of 6-8 mmHg (data not shown). Those subjects with higher blood pressure at baseline had a greater BP reduction (p<0.05), and this relationship was independent of % EWL (p=0.11-0.90). (data not shown) The magnitude of blood pressure reduction is not dependent on % EWL. For subjects with elevated blood pressure at baseline and those with greater than or equal to 9 hours of therapy were not dependent on changes in hypertensive medications (p=0.20-0.80).

Therapy of greater than or equal to 9 hours leads to reduction in blood pressure at 2 weeks prior to weight loss and reduction in blood pressure is greater for patients with high blood pressure at baseline. Reduction in blood pressure is not dependent on weight loss magnitude and is independent of weight loss. Blood pressure reduction is not associated with changes in hypertension medications based on the available data

Improved % EWL with increased duration of device usage supports a potential beneficial effect of vagal blockade on weight loss. In addition, the substantial improvement in blood pressure as early as two weeks after treatment and prior to any significant weight loss also supports this view. Lack of difference in % EWL in the treated vs. the control groups raises the question of whether the vagal manipulation and/or seemingly minor electrical input delivered to the vagal nerves in the control group for the safety and device diagnostic checks may actually have had an effect on vagal function.

The design of the clinical trial was intended to maintain a safe and eventually active device in both groups. In the control group the device was activated and consistently delivered electrical signals of much lower energy. The safety check algorithm in the control group delivered less than one thousandth of the input delivered in the treated group (for instance, if the device was worn for 10 h/day, the total charge delivered to the treated and control groups was 3.9 versus 0.0014 Coulombs, respectively). Preliminary experiments using the rat sciatic nerve model had suggested that these “safety checks” would have little or no effect on vagal blockade (unpublished data). A subsequent study (after reaching the 1 year data collection) was performed using the rat sciatic nerve model in 9 anesthetized rats to determine if the parameters utilized in the control group might have a neuromodulatory effect contributing to the unexpected weight loss in the control group. This preliminary study in rats using an accelerated model to mimic one hour in humans (1 min ON followed by 1 min OFF) showed that these electrical parameters led to a mean decrease of 31% in the amplitude of the compound action potential when evaluated 16 min later. (data not shown) The mean time of onset was 6 min after start of the control mode parameters and increased thereafter cumulatively. Longer-term studies are yet to be done to determine if this effect on compound action potentials increases further or lasts when the electrical stimulus is stopped. These observations suggest that electrical input to the vagal nerves for impedance and safety checks in the control group may have decreased vagal nerve excitability and may have contributed to the unexpected weight loss and decrease in blood pressure observed in the control group. The sensitivity of any given human participant in the trial to the amplitude necessary to induce weight loss through vagal blockade is unknown, but these data suggest the response may be variable.

Vagal modulation could increase satiety by an effect on the central nervous apparatus or by effects on the gut, such as a decrease in gastric emptying of solids by suppressing antral contractions, by inhibiting gastric receptive relaxation/accommodation, or by release of gut hormones that might increase postprandial satiety. Similarly, by decreasing pancreatic exocrine secretion, absorption of ingested food may have decreased; without any notable change in bowel habits or associated steatorrhea, this latter possibility is unlikely. Second, the progressive increase in % EWL loss in both groups (treated and control) with increasing duration of device use may represent better compliance among those more devoted to the study and more dedicated to losing weight, thereby representing an internally-biased group. Third, the study did include a program of dietary counseling, behavior modification, and exercise training, which may have increased the % EWL loss. Fourth, the inconvenience of wearing the external delivery device demands a more compliant subject, while a totally implantable delivery device is likely more attractive.

In summary, under the conditions of this EMPOWER study, we were unable to demonstrate any difference in the treated and control groups in % EWL. Greater weight loss with increasing device usage and decease in blood pressure independent of weight loss suggests that small electrical inputs delivered to vagal nerves in the control group for safety and impedance checks may have altered vagal excitability and thereby confounded the study.

In a more recent study double blind study in which the control patients were implanted but the vagus nerve did not receive any electrical signal, the treated patients demonstrated a statistically significant weight loss as compared to control patients at the 12 month time period. In the primary analysis (intent-to-treat) population (n=239), treatment patients achieved a 24.4% average EWL compared to 15.9% for sham control patients. This 8.5% difference demonstrated statistical superiority over sham control (p=0.002), but not super-superiority at the pre-specified 10% margin (p=0.705). In total, 52.5% of treatment patients had 20% or more EWL compared to 32.5% in the control group (p=0.004), and 38.3% of treatment patients had 25% or more EWL compared to 23.4% in the sham control group (p=0.02). While the respective co-primary endpoint targets of 55% and 45% were not met, the endpoint targets were within the 95% confidence intervals for the observed rates and therefore the observed rates were not significantly lower than these pre-specified rates. These efficacy data demonstrate VBLOC Therapy's positive effect on weight loss. In the per protocol group, which included only those patients who received therapy per the trial design (n=211), the treatment patients had an average 26.3% EWL compared to 17.3% for the sham control group (p=0.003). In total, 56.8% of treated patients achieved at least 20% EWL, which was above the pre-defined threshold of 55%, compared to 35.4% in the sham control group (p=0.004). 41.8% of patients also achieved at least 25% EWL in this population, which is slightly less than the predefined threshold of 45%, compared to 26.2% in the sham control group (p=0.03).

The rate of device-related serious adverse events was 3.1% for the treatment arm, significantly lower than the threshold of 15% (p<0.0001). The safety results also confirmed VBLOC Therapy had no adverse cardiovascular effect. An overall reduction in blood pressure and heart rate was also observed in the treatment arm. Approximately 93% of patients reached the 12 month assessment in the trial, consistent with a rigorously executed trial.

With the foregoing detailed description of the present invention, it has been shown how the objects of the invention have been attained. Modifications and equivalents of disclosed concepts such as those which might readily occur to one skilled in the art are intended to be included in the scope of the claims which are appended hereto.

In the sections of this application pertaining to teachings of the prior art, the specification from prior art patents is substantially reproduced for ease of understanding the embodiment of the present invention. For the purpose of the present application, the accuracy of information in those patents is accepted without independent verification. Any publications referred to herein are hereby incorporated by reference.

What is claimed is:

1. An apparatus for treating a condition associated with altered blood pressure comprising:
a) A first electrode adapted to be placed on a first target nerve or blood vessel selected from the group consisting of renal artery, renal nerve, celiac plexus, a splanchnic nerve, cardiac sympathetic nerves, and spinal nerves originating between T10 to L5;
b) an implantable neuroregulator connected to the electrodes and configured to deliver a first therapy program to the first target nerve or blood vessel, wherein the first therapy program delivers an electrical signal to the first target nerve or blood vessel intermittently with an on time and an off time multiple times in a day, wherein the first therapy program delivers an electrical signal treatment that has a frequency selected to down regulate neural activity on the first nerve or blood vessel during an on time and has an off time selected to provide for at least partial recovery of nerve function; and
c) an external coil, wherein the external coil is configured to communicate data and power signals to the neuroregulator and to communicate data to another programming device.

2. An apparatus according to claim 1, further comprising an additional electrode is adapted to be placed on a second target nerve or tissue selected from renal artery, renal nerve, vagus nerve, celiac plexus, a splanchnic nerve, cardiac sympathetic nerves, spinal nerves originating between T10 to L5, and glossopharyngeal nerve, tissue containing baroreceptors.

3. An apparatus for treating a condition associated with excess blood pressure comprising:
A first electrode adapted to be placed on a first target nerve or blood vessel selected from the group consisting of renal artery, renal nerve, vagus nerve, celiac plexus, a splanchnic nerve, cardiac sympathetic nerves, and spinal nerves originating between T10 to L5, and an additional electrode adapted to be placed on a second target nerve or tissue selected from renal artery, renal nerve, celiac plexus, a splanchnic nerve, cardiac sympathetic nerves, spinal nerves originating between T10 to L5, and glossopharyngeal nerve, tissue containing baroreceptors;
b) an implantable neuroregulator connected to the electrodes and configured to deliver a first therapy program to the first target nerve or blood vessel, wherein the first therapy program delivers an electrical signal to the first target nerve or blood vessel intermittently with an on time and an off time multiple times in a day, wherein the first therapy program delivers an electrical signal treatment that has a frequency selected to down regulate neural activity on the first nerve or blood vessel during an on time and has an off time selected to provide for at least partial recovery of nerve function; and
c) an external coil, wherein the external coil is configured to communicate data and power signals to the neuroregulator and to communicate data to another programming device.

4. An apparatus according to claim 1, further comprising
the implantable neuroregulator configured to deliver a third therapy program to the second target nerve or tissue, wherein the third therapy program delivers an electrical signal to second target nerve or blood vessel intermittently with an on time and an off time multiple times in a day, wherein the third therapy program delivers an electrical signal treatment that has a frequency to up regulate neural activity when the additional electrode is adapted to be placed on a second target nerve or tissue selected from a glossopharyngeal nerve, tissue containing baroreceptors, and combinations thereof.

5. An apparatus according to claim 1, further comprising a sensor operatively coupled to the implantable neuroregulator.

6. An apparatus of claim 5, wherein the sensor is operatively coupled to the implantable neuroregulator through a lead.

7. An apparatus of claim 5, wherein the sensor is implantable.

8. An apparatus of claim 5, wherein the sensor detects a parameter selected from the group consisting of blood pressure, heart rate, mean arterial pressure, hormones, and combinations thereof.

9. An apparatus of claim 5, wherein the implantable neuroregulator is configured to activate the first and/or third therapy program if the blood pressure exceeds a high blood pressure threshold.

10. An apparatus of claim 9, wherein the high blood pressure threshold is about 130 mm Hg systolic, 80 mmHg diastolic, or both.

11. An apparatus of claim 2, wherein the first electrode or additional electrode is adapted to be placed on a renal nerve or renal artery.

12. An apparatus of claim 3, wherein the first electrode is adapted to be placed on a vagus nerve.

13. An apparatus of claim 3, wherein the first electrode is adapted to be placed on a vagus nerve and the additional electrode is adapted to be placed on a glossopharyngeal nerve.

14. An apparatus of claim 3, wherein the first electrode is adapted to be placed on a vagus nerve and the additional electrode is adapted to be placed on a tissue with baroreceptors.

15. An apparatus of claim 2, wherein a first electrode is adapted to be placed on a renal nerve or artery and the additional electrode is adapted to be placed on a vagus nerve.

16. An apparatus of claim 3, wherein the first electrode is adapted to be placed on a vagus nerve and the additional electrode is adapted to be placed on a cardiac sympathetic nerve, a spinal sympathetic nerve or splanchnic nerve.

17. An apparatus of claim 1, wherein the neuroregulator is configured to deliver the electrical signal intermittently for a treatment period of no less than 9 hours and no more than 18 hours.

18. An apparatus of claim 1, wherein the downregulating signal has a frequency of about 200 to 5000 Hz.

19. An apparatus of claim 4, wherein the up regulating signal has a frequency of about 1 to 200 Hz.

20. An apparatus of claim 1, wherein the downregulating signal is applied at the same time as the upregulating signal.

21. An apparatus according claim 1, wherein the condition is hypertension, congestive heart failure, or chronic kidney disease.

22. A method of manufacturing an apparatus of claim 1 comprising:
a) Configuring the implantable neuroregulator to deliver a first therapy program to the first target nerve or blood vessel, wherein the first therapy program delivers an electrical signal to the first target nerve or blood vessel intermittently with an on time and an off time multiple times in a day, wherein the first therapy program delivers an electrical signal treatment that has a frequency selected to down regulate neural activity on the first nerve or blood vessel during an on time and has an off time selected to provide for at least partial recovery of nerve function;
b) Configuring the implantable neuroregulator to deliver a third therapy program to the second target nerve or tissue, wherein the third therapy program delivers an electrical signal to second target nerve or blood vessel intermittently with an on time and an off time multiple times in a day, wherein the third therapy program delivers an electrical signal treatment that has a frequency to up regulate neural activity; and
c) Configuring the implantable neuroregulator to operate in selectable multiple modes comprising a first mode comprising providing the first therapy program to the first and additional electrode, and a second mode comprising providing the first therapy program to the first electrode and the third therapy program to the additional electrode.

23. The method of claim 22, further providing a first electrode adapted to be placed on a first target nerve or blood vessel selected from the group consisting of renal artery, vagus nerve, renal nerve, vagus nerve, celiac plexus, a splanchnic nerve, cardiac sympathetic nerves, and spinal nerves originating between T10 to L5.

24. The method of claim 22, further providing an additional electrode adapted to be placed on a first target nerve or blood vessel selected from the group consisting of renal artery, renal nerve, vagus nerve, celiac plexus, a splanchnic nerve, cardiac sympathetic nerves, spinal nerves originating between T10 to L5, glossopharyngeal nerve, and tissue containing baroreceptors.

25. The method of claim 22, providing a sensor, wherein the sensor detects a parameter selected from the group consisting of blood pressure, heart rate, mean arterial pressure, hormones, and combinations thereof.

26. The method of claim 25, further comprising configuring the implantable neuroregulator to activate the first and/or third therapy program if the blood pressure exceeds a high blood pressure threshold.

27. The method according to claim 22, wherein the electrical signal of the first and third therapy program is selected for frequency, pulse width, amplitude, timing and ramp-up/ramp-down characteristics.

28. The method according to claim 27, wherein the first therapy program delivers an electrical signal to the target nerve or blood vessel of a frequency of about 200 Hz to 25 kHz.

29. The method of claim 22, wherein the first and third therapy programs are configured to be delivered during the same on time or at different on times.

30. A method of treating hypertension or congestive heart failure comprising:
a) selecting a drug for treating hypertension for a patient where effective dosages for treating hypertension for such a patient are associated with disagreeable side effects or inadequate blood pressure control; and b) treating a patient for hypertension with a concurrent treatment comprising:
i) applying an intermittent electrical treatment signal to a renal nerve or renal artery of the patient at multiple times per day and over multiple days with the block selected to down-regulate afferent and/or efferent neural activity on the nerve and with neural activity at least partially restoring upon discontinuance of said block; and
ii) administering said drug to the patient.

31. The method of claim 30,wherein the agent that improves blood pressure control is selected from the group consisting of a diuretic, ACE inhibitor, calcium channel blocker, beta blocker, alpha blocker and mixtures thereof.
